(19) United States
(12) Patent Application Publication
(10) Pub. No.: US 2007/0244133 A1
(43) Pub. Date: Oct. 18, 2007

(54) Thienopyrimidines and Thiazolopyrimidines for Use in Medicine

(76) Inventors: Justin Fairfield Bower, Cheshire (GB); Alan Wellington Faull, Cheshire (GB); Jon Winter, Cheshire (GB)

Correspondence Address: MORGAN LEWIS & BOCKIUS LLP, 1111 PENNSYLVANIA AVENUE NW, WASHINGTON, DC, 20004, US

(21) Appl. No.: 11/628,449

(22) PCT Filed: May 31, 2005
(86) PCT No.: PCT/GB05/02138

§ 371 Date: Dec. 4, 2006

(30) Foreign Application Priority Data
Jun. 4, 2004 (GB) 0412467.3

(51) Int. Cl. A61K 31/519; A61P 1/00; A61P 11/00; A61P 17/06; A61P 35/00; C07D 239/70; C07D 495/04; C07D 513/04

(52) U.S. Cl. 514/260.1; 544/255; 544/280

(57) ABSTRACT

The use of a compound of formula (I) or a pharmaceutically acceptable salt or solvate thereof, wherein X¹, X², X³, X⁴, R^{a}, p, R¹, Z, Y, R², R³ and R⁴ are as defined in the specification, in the preparation of a medicament for the treatment of C—C chemokine mediated conditions, such as inflammatory disease. Certain compounds of formula (I) are novel and these, together with their preparation are also described and claimed.

The present invention relates to pharmaceutical compositions which comprise compounds that act via antagonism of the CCR2b receptor for which MCP-1 is one of the known ligands, and so may be used to treat inflammatory disease which is mediated by these receptors. These compounds contain a bicyclic aromatic moiety. The invention further relates to novel compounds for use in the compositions, to processes for their preparation, to intermediates useful in their preparation and to their use as therapeutic agents.

Chemokines play an important role in immune and inflammatory responses in various diseases and disorders, including rheumatoid arthritis, chronic obstructive pulmonary disease, atherosclerosis and other autoimmune pathologies such as inflammatory bowel disease, diabetes, asthma and allergic diseases. Chemokines also have a role in angiogenesis and modulation of chemokines may be beneficial in the treatment of cancer. Chemokines are small secreted molecules belonging to a growing superfamily of 8-14 kDa proteins characterised by a conserved four cysteine motif. The chemokine superfamily can be divided into two main groups exhibiting characteristic structural motifs, the Cys-X-Cys (C—X—C) and Cys-Cys (C—C) families. These are distinguished on the basis of a single amino acid insertion between the NH-proximal pair of cysteine residues and sequence similarity.

The C—C chemokines include potent chemoattractants of monocytes and lymphocytes such as monocyte chemoattractant proteins 1-3 (MCP-1, MCP-2 and MCP-3), RANTES (Regulated on activation, Normal T expressed and Secreted), eotaxin and the macrophage inflammatory proteins 1 (and 1 (MIP-1α and MIP-1β).

The C—X—C chemokines include several potent chemoattractants and activators of neutrophils such as interleukin-8 (IL-8) and neutrophil-activating peptide 2 (NAP-2).

Studies have demonstrated that the actions of chemokines are mediated by subfamilies of G-protein coupled receptors, among which there are the receptors designated CCR1, CCR2, CCR2A, CCR2B, CCR3, CCR4, CCR5, CCR6, CCR7, CCR8, CCR9, CCR10, CXCR1, CXCR2, CXCR3, CXCR4, CXCR5 and CX3CR1. These receptors represent good targets for drug development since agents which modulate these receptors would be useful in the treatment of disorders and diseases such as those mentioned above.

U.S. Pat. No. 6,579,882 and US Patent Application No. 2001/0020030 describe a wide range of bicyclic compounds which are cell adhesion-inhibiting anti-inflammatory compounds.

The applicants have found a class of compounds containing a bicyclic moiety which have useful antagonism of the CCR2b receptor.

The present invention provides the use of a compound of formula (I) 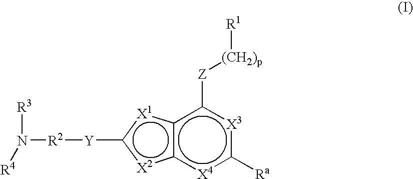
or a pharmaceutically acceptable salt, ester or amide thereof,
wherein X¹ or X² are selected from sulphur, nitrogen or CH, provided that at least one of X¹ or X² is sulphur or nitrogen;
one of X³ or X⁴ is nitrogen and the other is N or CH;
R^{a} is hydrogen, C₁₋₃alkyl, C₂₋₄alkenyl, C₂₋₄alkynyl, trifluoromethyl, halo, amino, C₁₋₃alkylamino, di-C₁₋₃alkylamino, C₁₋₄alkoxy, hydroxy, thioC₁₋₄alkyl, or cyclopropyl;
p is 0 or an integer selected from 1, 2, 3 or 4;
R¹ is hydrogen, or an optionally substituted cycloalkyl or optionally substituted aryl ring, wherein two substituents may be joined together to form an optionally substituted fused bicyclic ring, which may contain hetero atoms,
Z is oxygen or a group NR⁶ or —NR⁶C(O)— where R⁶ is hydrogen or C₁₋₆alkyl, or R⁶ is a C₂₋₆alkylene or C₂₋₆alkenylene group that is bonded to the ring R¹ to form a fused bicyclic ring system;
Y is a direct bond or a group, —O—, —C(O)—, —S(O)ₘ—, —NR⁸—, —NR⁸C(O)—, —C(O)NR⁸—, S(O)ₘNR⁸— or —NR⁸S(O)ₘ—, where m is 0, 1 or 2 and R⁸ is hydrogen or an optionally substituted C₁₋₄alkyl group,
R² is a direct bond, a C₁₋₁₀straight or branched alkylene group, which is optionally interposed with a group NR^{b} where R^{b} is hydrogen or a C₁₋₃methyl group; or R² together with R⁸ may form an optionally substituted cycloalkyl or heterocyclic ring,
R³ and R⁴ are independently selected from an optionally substituted C₁₋₁₀ alkyl group, an optionally substituted C₂₋₁₀ alkenyl group, an optionally substituted C₁₋₁₀ alkynyl group or an optionally substituted heterocyclic group,
or R³ and R⁴ together with the nitrogen atom to which they are attached form an optionally substituted heterocyclic ring, which optionally contains additional heteroatoms,
or R³ together with R² or R⁸ and the nitrogen atom(s) to which they are attached form an optionally substituted heterocyclic ring which optionally contains additional heteroatoms,
or R³ and R⁴ together with R² form an optionally substituted bridged ring structure, in the preparation of a medicament for the treatment of C—C chemokine mediated conditions.

The invention is related to the use of compounds in the treatment of diseases in which the chemokine receptor belongs to the C—C receptor subfamily, more preferably the target chemokine receptor is the CCR2 receptor.

CCR2 is a receptor for the Monocyte chemoattractant protein-1 (MCP-1). MCP-1 is a member of the chemokine family of pro-inflammatory proteins which mediate leukocyte chemotaxis and activation. MCP-1 is a C—C chemokine which is potent T-cell and monocyte chemoattractant. MCP-1 has been implicated in the pathophysiology of a large number of inflammatory diseases including rheumatoid arthritis, chronic obstructive pulmonary disease, atherosclerosis and inflammatory bowel disease.

MCP-1 acts through the CCR2 receptor. MCP-2, MCP-3 and MCP-4 may also act, at least in part, through this receptor. Therefore in this specification, when reference is made to “inhibition or antagonism of MCP-1” or “MCP-1 mediated effects” this includes inhibition or antagonism of MCP-2 and/or MCP-3 and/or MCP-4 mediated effects when MCP-2 and/or MCP-3 and/or MCP-4 are acting through the CCR2 receptor.

A compound of formula (I), or a pharmaceutically acceptable salt thereof, can be used in the preparation of medicaments for the treatment of:
(1) (respiratory tract)—obstructive diseases of the airways including: asthma, including bronchial, allergic, intrinsic, extrinsic, exercise-induced, drug-induced (including aspirin and NSAID-induced) and dust-induced asthma, both intermittent and persistent and of all severities, and other causes of airway hyper-responsiveness; chronic obstructive pulmonary disease (COPD); bronchitis, including infectious and eosinophilic bronchitis; emphysema; bronchiectasis; cystic fibrosis; sarcoidosis; farmer's lung and related diseases; hypersensitivity pneumonitis; lung fibrosis, including cryptogenic fibrosing alveolitis, idiopathic interstitial pneumonias, fibrosis complicating anti-neoplastic therapy and chronic infection, including tuberculosis and aspergillosis and other fungal infections; complications of lung transplantation; vasculitic and thrombotic disorders of the lung vasculature, and pulmonary hypertension; antitussive activity including treatment of chronic cough associated with inflammatory and secretory conditions of the airways, and iatrogenic cough; acute and chronic rhinitis including rhinitis medicamentosa, and vasomotor rhinitis; perennial and seasonal allergic rhinitis including rhinitis nervosa (hay fever); nasal polyposis; acute viral infection including the common cold, and infection due to respiratory syncytial virus, influenza, coronavirus (including SARS) and adenovirus.
(2) (bone and joints) arthritides associated with or including osteoarthritis/osteoarthrosis, both primary and secondary to e.g. congenital hip dysplasia; cervical and lumbar spondylitis, and low back and neck pain; rheumatoid arthritis and Still's disease; seronegative spondyloarthropathies including ankylosing spondylitis, psoriatic arthritis, reactive arthritis and undifferentiated spondarthropathy; septic arthritis and other infection-related arthopathies and bone disorders such as tuberculosis, including Potts' disease and Poncet's syndrome; acute and chronic crystal-induced synovitis including urate gout, calcium pyrophosphate deposition disease, and calcium apatite related tendon, bursal and synovial inflammation; Behçet's disease; primary and secondary Sjogren's syndrome; systemic sclerosis and limited scleroderma; systemic lupus erythematosus, mixed connective tissue disease, and undifferentiated connective tissue disease; inflammatory myopathies including dermatomyositits and polymyositis; polymalgia rheumatica; juvenile arthritis including idiopathic inflammatory arthritides of whatever joint distribution and associated syndromes, and rheumatic fever and its systemic complications; vasculitides including giant cell arteritis, Takayasu's arteritis, Churg-Strauss syndrome, polyarteritis nodosa, microscopic polyarteritis, and vasculitides associated with viral infection, hypersensitivity reactions, cryoglobulins, and paraproteins; low back pain; Familial Mediterranean fever, Muckle-Wells syndrome, and Familial Hibernian Fever, Kikuchi disease; drug-induced arthalgias, tendonititides, and myopathies.
(3) (skin) psoriasis, atopic dermatitis, contact dermatitis or other eczematous dermatoses, and delayed-type hypersensitivity reactions; phyto- and photodermatitis; seborrhoeic dermatitis, dermatitis herpetiformis, lichen planus, lichen sclerosus et atrophica, pyoderma gangrenosum, skin sarcoid, discoid lupus erythematosus, pemphigus, pemphigoid, epidermolysis bullosa, urticaria, angioedema, vasculitides, toxic erythemas, cutaneous eosinophilias, alopecia areata, male-pattern baldness, Sweet's syndrome, Weber-Christian syndrome, erythema multiforme; cellulitis, both infective and non-infective; panniculitis; cutaneous lymphomas, non-melanoma skin cancer and other dysplastic lesions; drug-induced disorders including fixed drug eruptions.
(4) (eyes) blepharitis; conjunctivitis, including perennial and vernal allergic conjunctivitis; iritis; anterior and posterior uveitis; choroiditis; autoimmune; degenerative or inflammatory disorders affecting the retina; ophthalmitis including sympathetic ophthalmitis; sarcoidosis; infections including viral, fungal, and bacterial.
(5) (gastrointestinal tract) glossitis, gingivitis, periodontitis; oesophagitis, including reflux; eosinophilic gastro-enteritis, mastocytosis, Crohn's disease, colitis including ulcerative colitis, proctitis, pruritis ani; coeliac disease, irritable bowel syndrome, and food-related allergies which may have effects remote from the gut (for example migraine, rhinitis or eczema).
(6) (abdominal) hepatitis, including autoimmune, alcoholic and viral; fibrosis and cirrhosis of the liver; cholecystitis; pancreatitis, both acute and chronic.
(7) (genitourinary) nephritis including interstitial and glomerulonephritis; nephrotic syndrome; cystitis including acute and chronic (interstitial) cystitis and Hunner's ulcer; acute and chronic urethritis, prostatitis, epididymitis, oophoritis and salpingitis; vulvo-vaginitis; Peyronie's disease; erectile dysfunction (both male and female).
(8) (Allograft rejection) acute and chronic following, for example, transplantation of kidney, heart, liver, lung, bone marrow, skin or cornea or following blood transfusion; or chronic graft versus host disease;
(9) (CNS) Alzheimer's disease and other dementing disorders including CJD and nvCJD; amyloidosis; multiple sclerosis and other demyelinating syndromes; cerebral atherosclerosis and vasculitis; temporal arteritis; myasthenia gravis; acute and chronic pain (acute, intermittent or persistent, whether of central or peripheral origin) including visceral pain, headache, migraine, trigeminal neuralgia, atypical facial pain, joint and bone pain, pain arising from cancer and tumor invasion, neuropathic pain syndromes including diabetic, post-herpetic, and HIV-associated neuropathies; neurosarcoidosis; central and peripheral nervous system complications of malignant, infectious or autoimmune processes.
(10) Other auto-immune and allergic disorders including Hashimoto's thyroiditis, Graves' disease, Addison's disease, diabetes mellitus, idiopathic thrombocytopaenic purpura, eosinophilic fasciitis, hyper-IgE syndrome, antiphospholipid syndrome.
(11) Other disorders with an inflammatory or immunological component; including acquired immune deficiency syndrome (AIDS), leprosy, Sezary syndrome, and paraneoplastic syndromes.
(12) (Cardiovascular); atherosclerosis, affecting the coronary and peripheral circulation; pericarditis; myocarditis, inflammatory and auto-immune cardiomyopathies including myocardial sarcoid; ischaemic reperfusion injuries; endocarditis, valvulitis, and aortitis including infective (e.g. syphilitic); vasculitides; disorders of the proximal and peripheral veins including phlebitis and thrombosis, including deep vein thrombosis and complications of varicose veins.
(13) (Oncology) treatment of common cancers including prostate, breast, lung, ovarian, pancreatic, bowel and colon, stomach, skin and brain tumors and malignancies affecting the bone marrow (including the leukaemias) and lymphoproliferative systems, such as Hodgkin's and non-Hodgkin's lymphoma; including the prevention and treatment of metastatic disease and tumour recurrences, and paraneoplastic syndromes.

The applicants have found that the introduction of a tertiary amine in the side chain is particularly advantageous in compounds of this type.

As used herein, the term “heteroatom” refers to non-carbon atoms such as oxygen, nitrogen or sulphur atoms. The term ‘alkyl’ when used either alone or as a suffix includes straight chain and branched structures. These groups may contain up to 10, preferably up to 6 and more preferably up to 4 carbon atoms. Similarly the terms “alkenyl” and “alkynyl” refer to unsaturated straight or branched structures containing for example from 2 to 10, preferably from 2 to 6 carbon atoms. Cyclic moieties such as cycloalkyl, cycloalkenyl and cycloalkynyl are similar in nature but have at least 3 carbon atoms. They may be bridged. Terms such as “alkoxy” and “alkanoyl” comprise alkyl moieties as defined above, attached to the appropriate functionality.

The term “halo” includes fluoro, chloro, bromo and iodo. References to aryl groups include aromatic carbocylic groups such as phenyl and naphthyl. The term “heterocyclyl” includes aromatic or non-aromatic rings, or partially unsaturated ring systems, for example containing from 4 to 20, suitably from 5 to 10 ring atoms, at least one of which is a heteroatom such as oxygen, sulphur or nitrogen. Rings may be mon- bi- or tri-cylic. Saturated ring systems may also contain bridges, in particular alkyl bridges. Examples of such groups include furyl, thienyl, pyrrolyl, pyrrolidinyl, imidazolyl, thiazolyl, tetrazolyl, oxazolyl, isoxazolyl, pyrazolyl, pyridyl, pyrimidinyl, pyrazinyl, pyridazinyl, triazinyl, quinolinyl, iosquinolinyl, quinoxalinyl, benzthiazolyl, benzoxazolyl, benzothienyl, benzofuranyl, tetrahydrofuryl, chromanyl, benzothienyl, piperidinyl, 1,2,3,4-tetrahydroquinolinyl, 1,2,3,4-tetrahydroisoquinolinyl, pyrazinyl, piperazinyl, pyrimidinyl, pyridazinyl, quinoxalinyl, quinazolinyl, cinnolinyl, indolyl, indolinyl, benzimidazolyl, pyrazolyl, indazolyl, oxazolyl, benzoxazolyl, isoxazolyl, isothiazolyl, morpholinyl, dioxolane, benzodioxolane, 4H-1,4-benzoxazinyl, 4H-1,4-benzothiazinyl, 1,2,3-triazolyl, 1,2,4-triazolyl, oxadiazolyl, furazanyl, thiadiazolyl, dibenzofuranyl, dibenzothienyl oxiranyl, oxetanyl, azetidinyl, piperidinyl, oxepanyl, oxazepanyl, tetrahydro-1,4-thiazinyl, 1,1-dioxotetrahydro-1,4-thiazinyl, homopiperidinyl, homopiperazinyl, dihydropyridinyl, tetrahydropyridinyl, dihydropyrimidinyl, tetrahydropyrimidinyl, tetrahydrothienyl, tetrahydrothiopyranyl or thiomorpholinyl.

“Heteroaryl” refers to those groups described above which have an aromatic character. The term “aralkyl” refers to aryl substituted alkyl groups such as benzyl.

Other expressions used in the specification include “hydrocarbyl” which refers to any structure comprising carbon and hydrogen atoms. For example, these may be alkyl, alkenyl, alkynyl, aryl, aralkyl, cycloalkyl, cycloalkenyl or cycloalkynyl.

Suitably one of X¹ or X² is sulphur and the other is nitrogen or CH.

In a particular embodiment of the invention, X¹ is sulphur and X² is nitrogen.

In another embodiment of the invention, X¹ is CH and X² is sulphur.

Suitably in the compounds of formula (I), both X³ and X⁴ are nitrogen.

Thus particular examples of compounds of formula (I) are compounds of formulae (IA)-(ID). 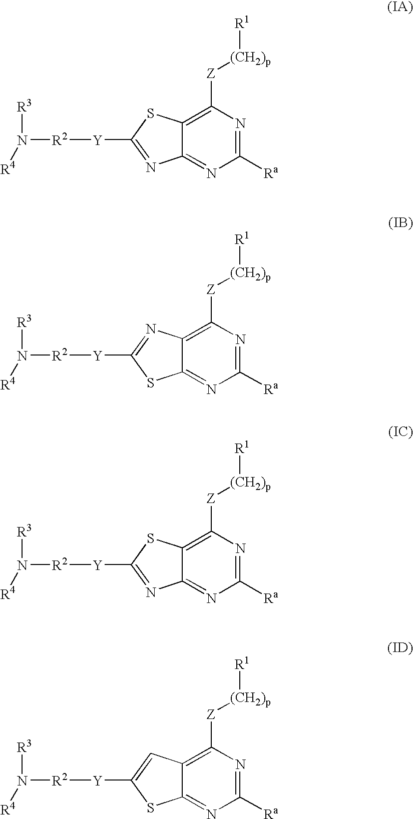
wherein R¹, R², R³, R⁴, R^{a}, Z, Y, and p are as defined in relation to formula (I).

R^{a} is suitably hydrogen or a small substituent such as methyl, trifluoromethyl or amino, and preferably R^{a} is hydrogen. When the compound of formula (I) is a compound of formula (IA), R^{a} may, in particular, be methyl.

In a further particular embodiment, p is 0 or 1, and in particular p is 0.

Where R¹ is hydrogen, p is suitably 1. Suitably however, R¹ is other than hydrogen.

In one embodiment of the invention, R¹ is optionally substituted aryl, and in particular optionally substituted phenyl or naphthyl. Suitably, R¹ is substituted phenyl.

Where R¹ is optionally substituted cycloalkyl, it is suitably an optionally substituted C₅₋₇cycloalkyl group, such as cyclohexyl.

Suitable optional substituents for cycloalkyl, aryl groups or heterocyclic groups R¹ include from 1 to 4, suitably from 1 to 3 groups selected from functional groups, hydrocarbyl groups such as alkyl groups, alkenyl, alkynyl groups or aralkyl groups, or heterocyclic groups.

As used herein, the term “functional group” refers to reactive substituents. They may comprise electron-donating or electron-withdrawing groups. Examples of such groups include halo, oxo, cyano, nitro, C(O)ₙR¹¹, OR¹¹, S(O)_{q}R¹¹, NR¹²R¹³, C(O)NR¹²R¹³, OC(O)NR¹²R¹³, —CH═NOR¹¹, —NR¹²C(O)ₙR¹¹, —NR¹¹CONR¹²R¹³, —N═CR¹²R¹³, S(O)_{q}NR¹²R¹³ or —NR¹²S(O)_{q}R¹¹ where R¹¹, R¹² and R¹³ are independently selected from hydrogen, optionally substituted hydrocarbyl or optionally substituted heterocyclyl, or R¹² and R¹³ together form an optionally substituted ring which optionally contains further heteroatoms such as S(O)_{q}—, oxygen and nitrogen, n is an integer of 1 or 2, q is 0 or an integer selected from 1, 2 or 3, and q′ is 0, 1 or 2. Where functional groups comprise S(O)_{q}NR¹²R¹³ or —NR¹²S(O)_{q}R¹¹, q is generally an integer of 1, 2 or 3, and suitably 1 or 2.

Suitable optional substituents for hydrocarbyl or heterocyclic groups R¹¹, R¹² and R¹³ include halo, (including perhaloalkyl such as trifluoromethyl), mercapto, hydroxy, alkoxy, oxo, heteroaryloxy, alkenyloxy, alkynyloxy, alkoxyalkoxy, aryloxy (where the aryl group may be substituted by halo, nitro, or hydroxy), cyano, nitro, amino, mono- or di-alkyl amino, alkylamido, oximino (for example hydroxyimino or alkyloxyimino) or S(O)_{q}R^{y} where q is as defined above and R^{y} is alkyl.

Particular substituents for R¹ include one or more groups selected from alkyl groups, in particular C₁₋₄alkyl groups such as methyl, C₂₋₄ alkenyl, or alkynyl groups such as ethynyl, benzyl, a saturated heterocyclic group such as tetrahydropyranyl, or a functional group as defined above. Particular functional groups which can form substituents on R¹ include halo, cyano, C(O)ₙR¹¹, OR¹¹ and S(O)_{q}R¹¹. Particular examples of R¹¹ are hydrogen, alkyl, or aryl, and in particular methyl or phenyl.

A particular example of n is 1. A particular example of q is 0.

Thus examples of substituents for R¹ are one or more halo groups (such as chloro or fluoro), hydroxy, methoxy, cyano, methyl, methylthio, acetyl, ethynyl, benzyl or phenylsulphonyl,

Examples of R¹ groups include groups (a)-(u)

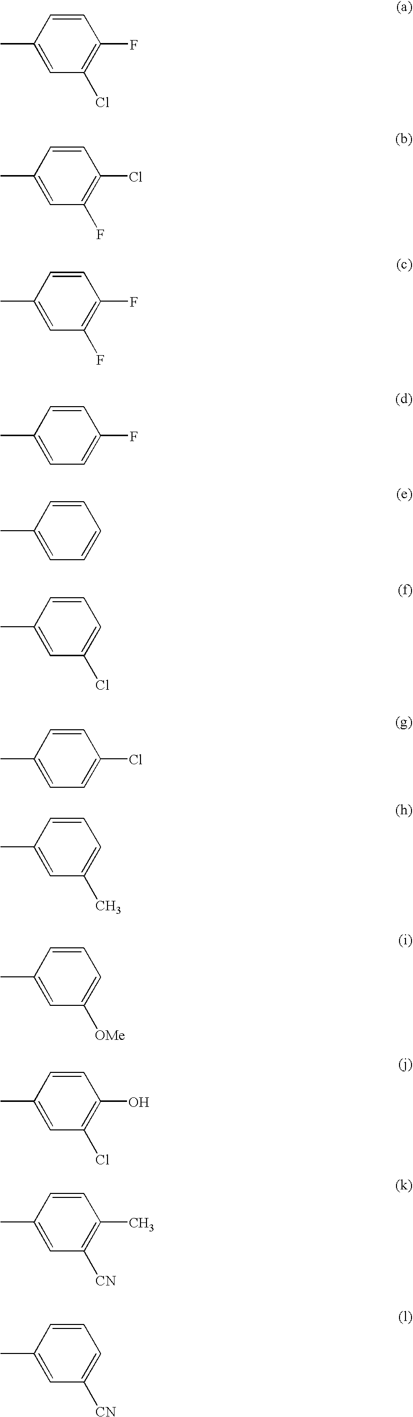

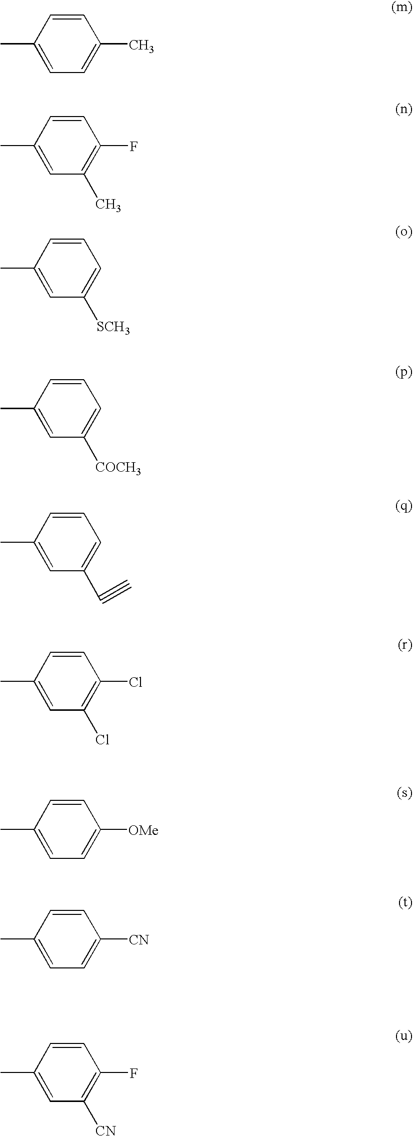

Suitably R¹ is substituted by one or two halo groups, which are preferably selected from chloro or fluoro.

A specific example of an R¹ group is 2-chloro-3-fluorophenyl.

Alternatively, two substituents on R¹ may be linked together to form an optionally substituted fused bicyclic ring system. Preferably the fused bicyclic ring is of formula (i)

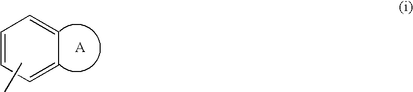

where A is an optionally substituted 4-7 membered ring which may contain one or more heteroatoms. Any substitutents on R¹ as described above, may be located on the ring A of the R¹ group. Particularly suitable optional substituents for the ring A include functional groups, heterocyclic groups or hydrocarbyl groups such as alkyl or aralkyl groups. The ring A may be saturated or unsaturated. When unsaturated, it may be aromatic in character. Suitably ring A forms a fused five or six membered ring.

Preferably ring A includes at least one heteroatom

Particular examples of bicyclic groups R¹ include groups of sub-formulae (v)-(f)

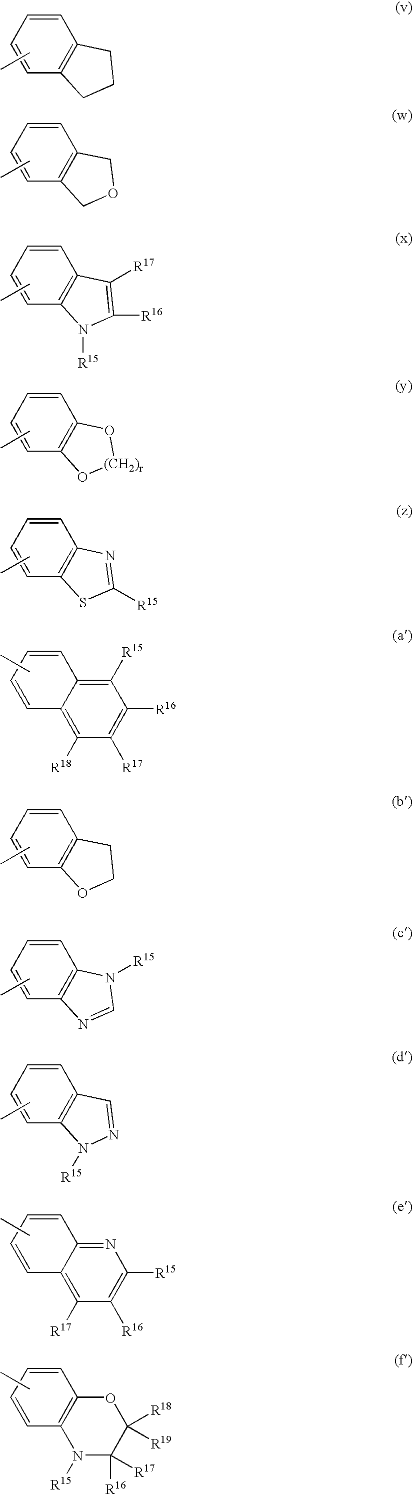

where r is 1, 2 or 3, and R¹⁵, R¹⁶, R¹⁷, R¹⁸ and R¹⁹ are independently selected from hydrogen or R¹ substituents as described above. In particular, where R¹⁵, R¹⁶, R¹⁷, R¹⁸ and R²⁰ are other than hydrogen, they are selected from alkyl such as methyl, methoxy, benzyl, piperidinyl, or phenylsulphonyl, or where two of R¹⁶, R¹⁷, R¹⁸ and R¹⁹ are on the same carbon atom, they may form an oxo substituent. Particular examples of such groups are illustrated hereinafter.

Suitably Z is a group NR⁶ where R⁶ is as defined above.

In a particular embodiment, R⁶ is hydrogen or C₁₋₃alkyl, such as methyl. Preferably R⁶ is hydrogen.

Where R⁶ is a C₂₋₆alkylene or C₂₋₆alkenylene group that is bonded to the ring R¹ to form a fused bicyclic ring system, it is suitably linked at the ortho position of the R¹ ring. Suitably, it contains from 2-4 carbon atoms. In particular, in this case, p is 0 and R⁶ is —(CH₂)₂—. Thus a particular example of such a group -Z-(CH₂)ₚ—R¹ is a group of sub-formula (aa)

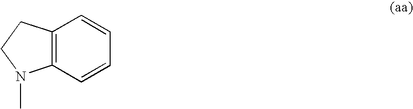

Particular examples of Y include a group, —O—, —C(O)—, —NR⁸—, —NR⁸C(O)— or —C(O)NR⁸—, where R⁸ is hydrogen or a C₁₋₄alkyl group such as methyl. In particular, any R⁸ groups are hydrogen or methyl. For the avoidance of doubt, the left hand side of the Y groups listed herein are linked to the R² group in formula (I), and the right hand side, as shown herein is linked to the bicyclic core ring.

In particular, Y is selected from —O—, —C(O)—, —NH—, —NHCO—, —N(CH₃)C(O)—, or —CONH—.

In one embodiment, Y is selected from —O— or —NH—.

In another embodiment Y is —NR⁸C(O)—, such as —NHC(O)—. Therefore the compound of formula (I) can be represented as follows formula (IE): 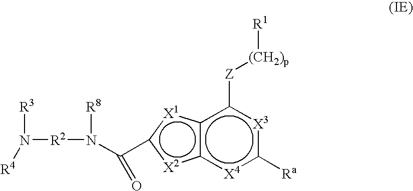
wherein R¹, R², R³, R⁴, R^{a}, Z, X¹, X², X³, X⁴ and p are as defined in relation to formula (I).

Where R⁸ is an optionally substituted alkyl group, suitable optional substituents include functional groups as defined above. Preferably R⁸ is unsubstituted.

In one embodiment of the invention, R² is a direct bond.

In an alternative embodiment, R² is a C₁₋₆straight or branched alkylene group, in particular, a C₂₋₃alkylene group.

Where R² is an alkylene chain which is interposed by a group NR^{b}, this group will not be at the end position of the chain. Preferably R^{b} is hydrogen.

Where R³ or R⁴ comprises an optionally substituted C₁₋₁₀ alkyl group, an optionally substituted C₂₋₁₀ alkenyl group, an optionally substituted C₂₋₁₀ alynyl group or an optionally substituted heterocyclic group, suitable optional substituents include functional groups, such as cyano, oxo, carboxy, cycloalkyl groups, aryl groups or heterocyclic groups where any cycloalkyl, aryl or heterocyclic substituents may themselves be optionally substituted by one or more functional groups, optionally substituted hydrocarbyl groups such as optionally substituted alkyl, or heterocyclic groups.

In a particular embodiment R³ or R⁴ are optionally substituted C₁₋₁₀alkyl groups.

Suitably R³ and/or R⁴ is methyl, ethyl, n-propyl, iso-propyl, n-butyl, n-pentyl or n-hexyl, and in particular methyl, ethyl or iso-propyl.

When groups R³ or R⁴ have a substituent which is a functional group, particular examples include cyano, C(O)ₙR¹¹ such as carboxy or methyl carboxylate, OR¹¹ such as hydroxy or methoxy, or S(O)_{q}R¹¹ such as thioC₁₋₃alkyl, for instance thiomethyl, or methylsulphonyl where n, q and R¹¹ are as defined above. In particular R¹¹ in this instance is selected from heterocyclic such as morpholino, or aryl such as phenyl.

In particular, where R³ or R⁴ are alkyl groups, they are optionally substituted by a heterocyclic group which may itself be optionally substituted. Particular examples of heterocyclic groups include furyl, tetrahydrofuryl thienyl, pyrrolyl, oxazolyl, isoxazolyl, thiazolyl, thiadiazolyl, isothiazolyl, imidazolyl, pyrazolyl, pyrrolidinyl, imidazolyl, pyridyl, pyrimidinyl, oxanyl, indolyl, quinolyl, isoquinolyl, piperidinyl, piperazinyl, dioxolanyl, benzo-1,3-dioxolyl, 2,3-dihydroindole, or thiiranyl.

In addition, R³ or R⁴ may comprise an alkyl group that is optionally substituted by an aryl such as phenyl, or cycloalkyl group such as cyclopropyl group, either of which may themselves be optionally substituted.

Where these aryl, cycloalkyl or heterocyclic substituents on R³ and R⁴ are themselves substituted, those substituents are suitably selected from C₁₋₃alkyl groups which optionally carry such a functional group as a substituent, or functional groups as defined above. Particular functional groups in this case include halo such as fluoro, cyano, oxo (where the ring is at least partially unsaturated) C(O)ₙR¹¹ such as carboxy or methyl carboxylate, OR¹¹ such as hydroxy or methoxy, or S(O)_{q}R¹¹ such as thioC₁₋₃alkyl, for instance thiomethyl, or methylsulphonyl where n, q and R¹¹ are as defined above,

In an alternative embodiment, R³ and R⁴ together with the nitrogen atom to which they are attached form an optionally substituted heterocyclic ring, which optionally contains additional heteroatoms. In particular, these rings are saturated rings. Examples of these are compounds of formula (I) where formula R⁴R³N— comprise a group of sub-formula (xx)-(xxv).

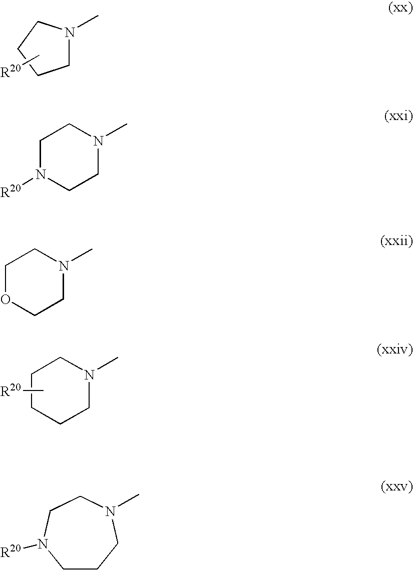

where R²⁰ is hydrogen or a substituent.

Suitable substituents R²⁰ include alkyl, in particular C₁₋₄alkyl such as methyl, aralkyl such as benzyl, optionally substituted heterocyclic groups, in particular saturated heterocyclic groups such as pyrrolidinyl or piperidinyl which may themselves be optionally substituted, and functional groups such as cyano, —NR¹²R¹³, C(O)ₙR¹¹, OR¹¹, or S(O)_{q}R¹¹ where n, q, R¹¹R¹² and R¹³ are as defined above. Particular functional groups C(O)ₙR¹¹ include carboxy or methyl carboxylate. Particular functional groups OR¹¹ are hydroxy or methoxy. Particular functional groups S(O)_{q}R¹¹ are thioC₁₋₃alkyl, for instance thiomethyl, or methylsulphonyl, as well as phenylsulphonyl.

When R²⁰ is a heterocyclic group, it may be optionally substituted by a functional group, in particular a functional group as listed above for R²⁰.

In yet another embodiment, R³ together with R² or R⁸ and the nitrogen atom(s) to which they are attached form an optionally substituted heterocyclic ring which optionally contains additional heteroatoms. Where R³ together with R² together with the nitrogen atom to which they are attached forms a ring, the attachment may take place at any suitable carbon atom within the R² chain, but is suitably at the R² carbon which is directly adjacent to the group Y. Thus, suitable examples of the group of sub-formula (x) 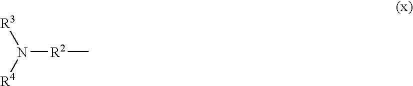
include groups of sub-formula (bb) or (cc) 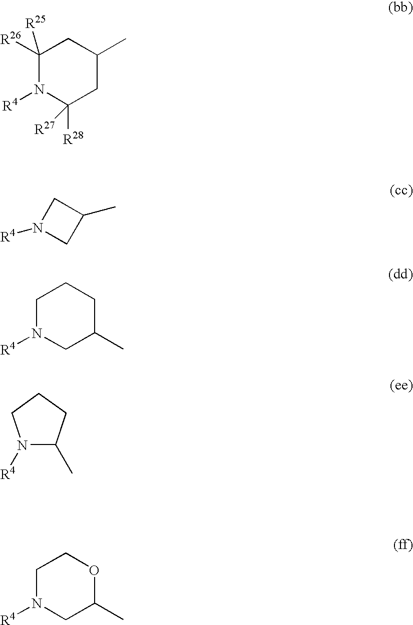
where R⁴ is as defined above, and R²⁵, R²⁶, R²⁷ and R²⁸ are independently selected from hydrogen or C₁₋₃alkyl such as methyl. Preferably R²⁵, R²⁶, R²⁷ and R²⁸ are all hydrogen, or all methyl, and most preferably, they are all hydrogen.

A particularly preferred group of (x) is a group of formula (bb) above.

Thus is a particular embodiment, the invention provides the use of a compound of formula IF 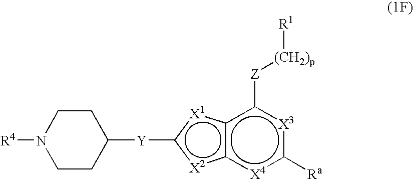
where R¹, R^{a}, R⁴, Y, X¹, X², X³, X⁴, Z and p are as defined above.

Particular examples of groups R⁴ in the groups of sub-formula (bb) to (ff) include those listed in Table 1.

TABLE 1

| Designation | R⁴ |
|---|---|
| 1a | —(CH₂)₂CH₃ |
| 1b | —(CH₂)₂OCH₃ |
| 1c | —CH₃ |
| 1d | 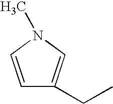 |
| 1e | 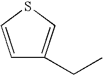 |
| 1f | 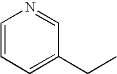 |
| 1g | 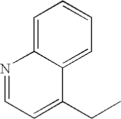 |
| 1h | —(CH₂)₃CH₃ |
| 1i | —CH₂CH═C(CH₃)₂ |
| 1j | 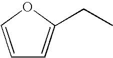 |
| 1k | 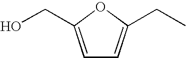 |
| 1l | 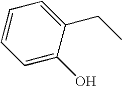 |
| 1m | 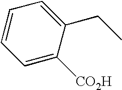 |
| 1n | 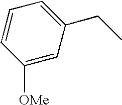 |
| 1o | 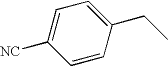 |
| 1p | 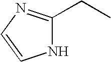 |
| 1q | 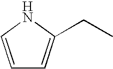 |
| 1r | 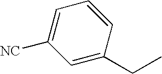 |
| 1s | 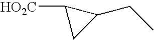 |
| 1t | 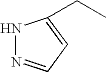 |
| 1u | 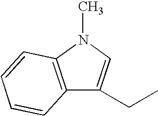 |
| 1v | 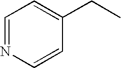 |
| 1w | 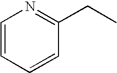 |
| 1x | 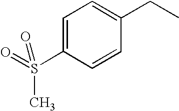 |
| 1y | 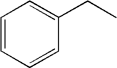 |
| 1z | 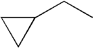 |
| 2a | 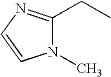 |
| 2b | 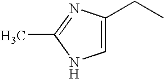 |
| 2c | 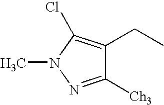 |
| 2d | 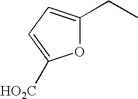 |
| 2e | —(CH₂)₂OH |
| 2f | 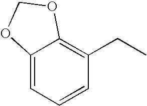 |
| 2g | 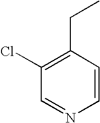 |
| 2h | —(CH₂)₅CO₂CH₃ |
| 2i | 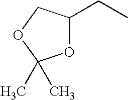 |
| 2j | 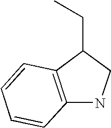 |
| 2k | 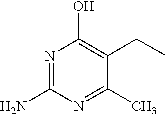 |
| 2l | 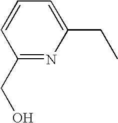 |
| 2m | 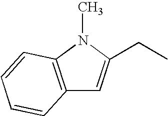 |
| 2n | 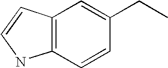 |
| 2o | 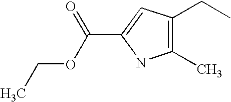 |
| 2p | 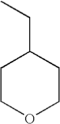 |
| 2q | 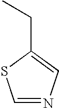 |
| 2r | 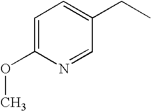 |
| 2s | 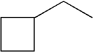 |
| 2t | 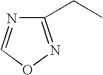 |
| 2u | 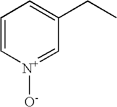 |
| 2v | 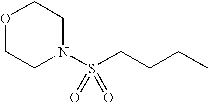 |
| 2w | 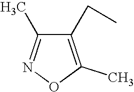 |
| 2x | 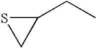 |
| 2y | 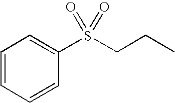 |
| 2z | 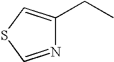 |
| 3a | —CH(CH₃)C≡CH |
| 3b | 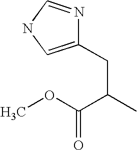 |
| 3c | CH(CH₃)C(O)N(CH₂CH₃)₂ |
| 3d | 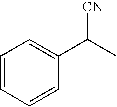 |
| 3e | —CH₂OCH₃ |
| 3f | 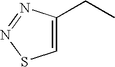 |
| 3g | —CH₂CH₃ |
| 3h | 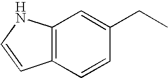 |
| 3i | 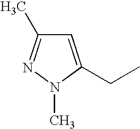 |
| 3j | —CH(CH₃)₂ |
| 3k | —CH(CH₃)C(O)CH₃ |
| 3l | 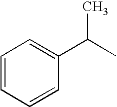 |
| 3m | 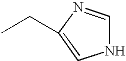 |
| 3n | 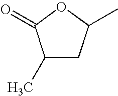 |
| 3o | 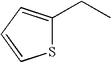 |
| 3p | 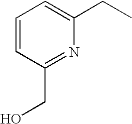 |
| 3q | 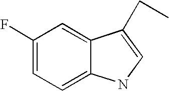 |
| 3r | 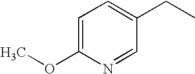 |
| 3s | —N(CH₃)₂ |
| 3t | 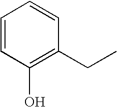 |
| 3u | 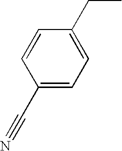 |
| 3v | 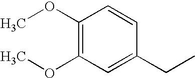 |
| 3w | —(CH₂)₂CH(CH₃)₂ |
| 3x | 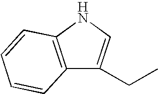 |
| 3y | 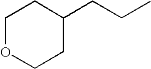 |
| 3z | 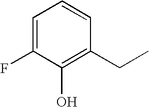 |
| 4a | 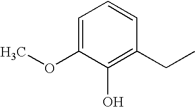 |
| 4b | 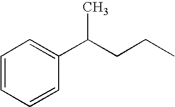 |
| 4c | 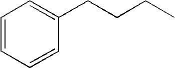 |
| 4d | 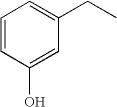 |
| 4e | 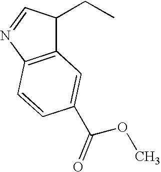 |
| 4f | 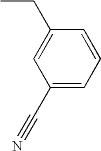 |
| 4g | 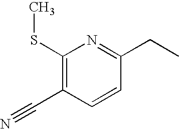 |
| 4h | 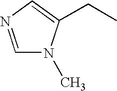 |
| 4i | 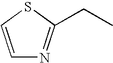 |
| 4j | 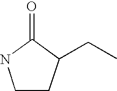 |

In a particularly preferred embodiment, the group R⁴ comprises alkyl substituted with heterocyclic group, which is optionally substituted as described above.

In another preferred embodiment, R⁴ is an alkyl group substituted with a substituted aryl group such as a substituted phenyl, wherein the substituents are as described above.

In yet another embodiment, R⁴ is a group S(O)_{q}R¹¹ where q and R¹¹ are as defined above.

Where R³ together with R⁸ and the nitrogen atoms to which they are attached form an optionally substituted heterocyclic ring which contains additional heteroatoms, suitable examples of the group of sub-formula (y) 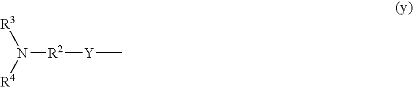
include groups of sub-formulae (g′) 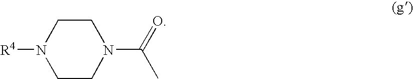
A particular example of such a group is 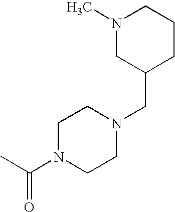
When R³ and R⁴ together with R² form an optionally substituted bridged ring structure, a particular structure is of formula (h′) 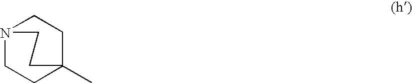
Suitable optional substituents are those described above for alkyl groups R³ or R⁴.

Novel compounds of formula (I) form a further aspect of the invention.

In a particular embodiment, the present invention provides a compound of formula (IG) 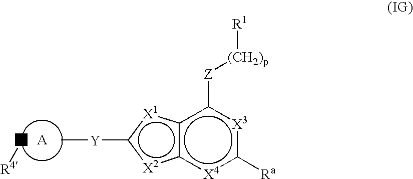
or a pharmaceutically acceptable salt, ester or amide thereof,
wherein X¹, X², X³, X⁴, R^{a}, p, R¹, Z and Y are as defined in relation to formula (I), the ring A is an optionally substituted heterocyclic ring which optionally contains further heteroatoms, and R^{4′} is a substituted C₁₋₁₀ alkyl group, provided that at least one substituent on the group R^{4′} is selected from optionally substituted heterocyclyl, substituted aryl, a cycloalkyl group, a group C(O)R¹¹ or a group S(O)_{q}R¹¹ where q and R¹¹ are as defined above.

Suitably the ring A is a saturated ring. Suitable optional substituents for ring A are as described above for R² and R³.

In particular the ring A-R^{4′} is a group selected from (bb), (cc), (dd), (ee) and (ff) above, and in particular is a group (bb).

Suitable substituents for the heterocyclic or aryl substituent on R^{4′} are C₁₋₃alkyl groups which optionally carry such a functional group as a substituent, or functional groups as defined above. Particular functional groups in this case include halo such as fluoro, cyano, oxo (where the ring is at least partially unsaturated) C(O)ₙR¹¹ such as carboxy or methyl carboxylate, OR¹¹ such as hydroxy or methoxy, or S(O)_{q}R¹¹ such as thioC₁₋₃alkyl, for instance thiomethyl, or methylsulphonyl where n, q and R¹¹ are as defined above.

Where R^{4′} carries a C(O)R¹¹ substituent, R¹¹ is suitably as defined above, but in particular is methyl.

Where R^{4′} carries a substituent S(O)_{q}R¹¹, q is suitably 2 and R¹¹ is as defined above. In particular R¹¹ is a heterocyclic or aryl group.

In an alternative embodiment, the invention provides compounds of formula (IH) 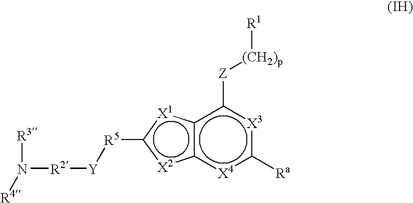
or a pharmaceutically acceptable salt, ester or amide thereof,
wherein X¹, X², X³, X⁴, R^{a}, p, R¹, Z and Y are as defined in relation to formula (I) R^{2′} is a C₁₋₁₀straight or branched alkylene group, which is optionally interposed with a group NR^{b} where R^{b} is hydrogen or a C₁₋₃methyl group; or R^{2′} together with any R⁸ group present in Y may form an optionally substituted cycloalkyl or heterocyclic ring, and R^{3′} and R^{4″} together with the nitrogen atom to which they are attached form a substituted heterocyclic ring, which optionally contains additional heteroatoms.

In particular, the rings formed by R^{3″} and R^{4″} are saturated rings. They are suitably substituted by any of the groups listed above as possible substituents for alkyl groups R³ and R⁴. In particular, R^{3″} and R^{4″} together form a group of sub-formula (xx)-(xxv) as defined above.

In yet a further embodiment, the invention provides a compound of formula (IJ) 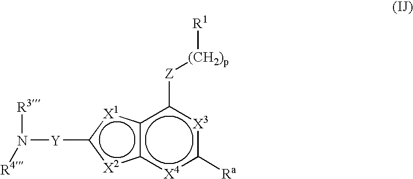
or a pharmaceutically acceptable salt, ester or amide thereof,
wherein X¹, X², X³, X⁴, R^{a}, p, R¹, Z, and Y are as defined in relation to formula (I) R^{3′″} and R^{4′″} together with the nitrogen atom to which they are attached form a heterocyclic ring, which optionally contains additional heteroatoms, and which is substituted by at least one group selected from (a) alkyl substituted by an optionally substituted heterocyclyl, (b) alkyl substituted by a substituted aryl group, (c) alkyl substituted by a cycloalkyl group, (d) a group C(O)R¹¹ or (e) a group S(O)_{q}R¹¹ where q and R¹¹ are as defined above.

Suitable substituents for the heterocyclic or aryl groups in (a) and (b) are C₁₋₃alkyl groups which optionally carry such a functional group as a substituent, or functional groups as defined above. Particular functional groups in this case include halo such as fluoro, cyano, oxo (where the ring is at least partially unsaturated) C(O)ₙR¹¹ such as carboxy or methyl carboxylate, OR¹¹ such as hydroxy or methoxy, or S(O)_{q}R¹¹ such as thioC₁₋₃alkyl, for instance thiomethyl, or methylsulphonyl where n, q and R¹¹ are as defined above.

Where (d) applies, the group R¹¹ is suitably as defined above, but in particular is methyl.

Where (e) applies, q is suitably 2 and R¹¹ is as defined above. In particular R¹¹ is a heterocyclic or aryl group.

A further embodiment of the invention provides a compound of formula (IK) 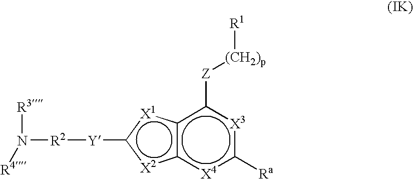
or a pharmaceutically acceptable salt, ester or amide thereof,
wherein X¹, X², X³, X⁴, R^{a}, p, R¹ and Z are as defined in relation to formula (I)
Y′ is a group, —NR^{8′}—, —NR^{8′}C(O)—, —C(O)NR^{8′}—, S(O)ₘNR^{8′} or —NR^{8′}S(O)ₘ—, where m is 0, 1 or 2
and R^{2′} together with R^{8′} forms an optionally substituted cycloalkyl or heterocyclic ring, R^{3″″} and R^{4″″} are independently selected from a substituted C₁₋₁₀ alkyl group (provided that at least one substitutent is other than hydroxy), an optionally substituted C₂₋₁₀ alkenyl group, an optionally substituted C₁₋₁₀ alkynyl group or an optionally substituted heterocyclic group,
or R^{3″″} and R^{41″} together with the nitrogen atom to which they are attached form an optionally substituted heterocyclic ring, which optionally contains additional heteroatoms.

In this embodiment, suitable groups R^{3′″} and R^{4′″} as well as substituents therefore are as described above in relation to R³ and R⁴.

In yet a further embodiment, the invention provides compounds of formula (IL) 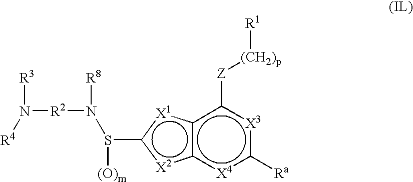
or a pharmaceutically acceptable salt, ester or amide thereof,
wherein X¹, X², X³, X⁴, R^{a}, p, R¹, Z, R², R³, R⁴, R⁸ and mare as defined in relation to formula (I).

Further embodiments of the invention include compounds of formula (IM) 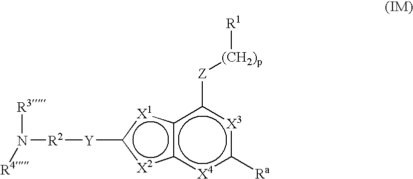
or a pharmaceutically acceptable salt, ester or amide thereof,
wherein X¹, X² X³, X⁴, R^{a}, p, R¹, Z, Y and R² are as defined in relation to formula (I) and R^{3′″″} and R^{4′″″} are independently selected from an optionally substituted C₁₋₁₀ alkyl group, an optionally substituted C₂₋₁₀ alkenyl group, an optionally substituted C₁₋₁₀ alkynyl group or an optionally substituted heterocyclic group, provided that at least one of R^{3′″″} or R^{4′″″} is other than optionally substituted alkyl.

Suitable examples of groups R^{3′″″} and R^{4′″″} are as described above in relation to formula R³ and R⁴.

In yet a further embodiment, the invention provides a compound of formula (IN) 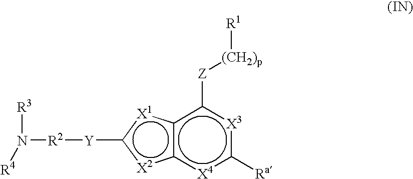
or a pharmaceutically acceptable salt, ester or amide thereof,
wherein X¹, X² X³, X⁴, R³, R⁴ p, R¹, Z, Y and R² are as defined in relation to formula (I), and R^{a′} is C₂₋₄alkenyl, C₂₋₄alkynyl, trifluoromethyl, or cyclopropyl.

In a further embodiment, the invention provides a compound of formula (IP) 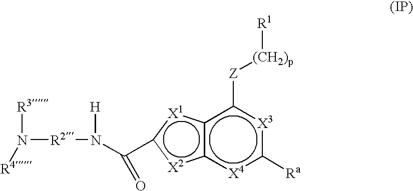
where R¹, p, Z, R^{a}, X¹, X², X³ and X⁴ are as defined in relation to formula (I), R^{2′″} is an alkylene group, which together with R^{3′″″″} and the nitrogen atom to which they are attached form a heterocyclic ring, and R^{4′″″″} is a heterocyclic group which is substituted by at least one substituted alkyl group, and which optionally contains further substituents.

In particular, compounds of formula (IP) are compounds of formula (IPa) 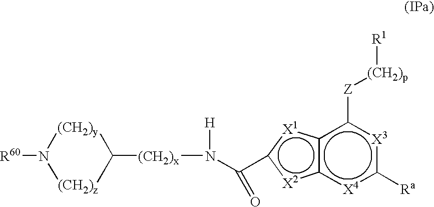
where R¹, p, Z, R^{a}, X¹, X², X³ and X⁴ are as defined in relation to formula (I), and R⁶⁰ is a substituted C₁₋₁₀ alkyl group, an optionally substituted C₂₋₁₀ alkenyl group, an optionally substituted C₁₋₁₀ alkynyl group or an optionally substituted heterocyclic group;
x is 0, 1 or 2; y and z are independently selected from 0, 1, 2, 3, 4 or 5, provided that y+z is in the range of 2 to 7.

Suitable substituents for alkyl groups R⁶⁰ and optional substitutents for alkenyl, alkynyl or heterocyclic groups R⁶⁰ include functional groups, such as cyano, oxo, carboxy, cycloalkyl groups, aryl groups or heterocyclic groups where any cycloalkyl, aryl or heterocyclic substituents may themselves be optionally substituted by one or more functional groups, optionally substituted hydrocarbyl groups such as optionally substituted alkyl, or heterocyclic groups.

In particular, R⁶⁰ is a substituted alkyl group in particular a substituted methyl group.

In particular, R⁶⁰ is substituted by a heterocyclic group which may itself be optionally substituted. Particular examples of heterocyclic groups include furyl, tetrahydrofuryl thienyl, pyrrolyl, oxazolyl, isoxazolyl, thiazolyl, thiadiazolyl, isothiazolyl, imidazolyl, pyrazolyl, pyrrolidinyl, imidazolyl, pyridyl, pyrimidinyl, oxanyl, indolyl, quinolyl, isoquinolyl, piperidinyl, piperazinyl, dioxolanyl, benzo-1,3-dioxolyl, 2,3-dihydroindole, or thiiranyl.

In addition, R⁶⁰ may comprise an alkyl group that is optionally substituted by an aryl such as phenyl, or cycloalkyl group such as cyclopropyl group, either of which may themselves be optionally substituted.

Where these aryl, cycloalkyl or heterocyclic substituents on R⁶⁰ are themselves substituted, those substituents are suitably selected from C₁₋₃alkyl groups which optionally carry such a functional group as a substituent, or functional groups as defined above. Particular functional groups in this case include halo such as fluoro, cyano, oxo (where the ring is at least partially unsaturated) C(O)ₙR¹¹ such as carboxy or methyl carboxylate, OR¹¹ such as hydroxy or methoxy, or S(O)_{q}R¹¹ such as thioC₁₋₃alkyl, for instance thiomethyl, or methylsulphonyl where n, q and R¹¹ are as defined above.

Suitably in formula (IPa), x is 0 of 1. Suitably y and z are both 2. Alternatively, one of y or z is 0 and the other is 4.

Suitable pharmaceutically acceptable salts of compounds of formula (I) include are base salts such as an alkali metal salt for example sodium, an alkaline earth metal salt for example calcium or magnesium, an organic amine salt for example triethylamine, morpholine, N-methylpiperidine, N-ethylpiperidine, procaine, dibenzylamine, N,N-dibenzylethylamine or amino acids for example lysine. In another aspect, where the compound is sufficiently basic, suitable salts include acid addition salts such as methanesulfonate, fumarate, hydrochloride, hydrobromide, citrate, maleate and salts formed with phosphoric and sulphuric acid. There may be more than one cation or anion depending on the number of charged functions and the valency of the cations or anions. A preferred pharmaceutically acceptable salt is a sodium salt.

An in vivo hydrolysable ester of a compound of the formula (I) containing carboxy or hydroxy group is, for example, a pharmaceutically acceptable ester which is hydrolysed in the human or animal body to produce the parent acid or alcohol.

Suitable pharmaceutically acceptable esters for carboxy include C₁₋₆alkyl esters such as methyl or ethyl esters, C₁₋₆alkoxymethyl esters for example methoxymethyl, C₁₋₆alkanoyloxymethyl esters for example pivaloyloxymethyl, phthalidyl esters, C₃₋₈cycloalkoxy-carbonyloxyC₁₋₆alkyl esters for example 1-cyclohexylcarbonyloxyethyl; 1,3-dioxolen-2-onylmethyl esters for example 5-methyl-1,3-dioxolen-2-onylmethyl; and C₁₋₆alkoxycarbonyloxyethyl esters for example 1-methoxycarbonyloxyethyl and may be formed at any carboxy group in the compounds of this invention.

An in vivo hydrolysable ester of a compound of the formula (I) containing a hydroxy group includes inorganic esters such as phosphate esters and α-acyloxyalkyl ethers and related compounds which as a result of the in vivo hydrolysis of the ester breakdown to give the parent hydroxy group. Examples of α-acyloxyalkyl ethers include acetoxymethoxy and 2,2-dimethylpropionyloxymethoxy. A selection of in vivo hydrolysable ester forming groups for hydroxy include alkanoyl, benzoyl, phenylacetyl and substituted benzoyl and phenylacetyl, alkoxycarbonyl (to give alkyl carbonate esters), dialkylcarbamoyl and N-(dialkylaminoethyl)-N-alkylcarbamoyl (to give carbamates), dialkylaminoacetyl and carboxyacetyl.

A suitable value for an amide includes, for example, a N—C₁₋₆alkyl and N,N-di-(C₁₋₆alkyl)amide such as N-methyl, N-ethyl, N-propyl, N,N-dimethyl, N-ethyl-N-methyl or N,N-diethylamide.

Particular compounds of formula (I) are listed below in Tables 2, 3 and 4.

### Table 2

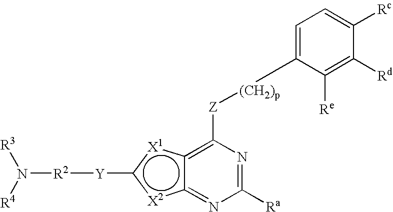

The chemical routes used to synthesise the Examples and certain intermediates in their preparation are designated A-G and described after the table of Examples. Where an example includes a reference to two such schemes, both were employed sequentially in its preparation. Also as shown in the table below, the groups Y are illustrated so that the left hand side of the molecule as shown is attached to R² and the right hand side is attached to the ring. Similarly, where applicable, the left hand side of the Z molecule as shown is attached to the ring, and the right hand side is attached to the (CH₂)ₚ group. Finally, when R^{d} and R^{e} form a ring, unless otherwise indicated, the left hand side of the molecule as shown is attached at the R^{d} position and the right hand side is linked at the R^{c} position in the above formula.

| No. | R⁴R³NR² | Y | X¹ | X² | R^{a} | p | Z | R^{e} | R^{d} | R^{c} |
|---|---|---|---|---|---|---|---|---|---|---|
| 1 | 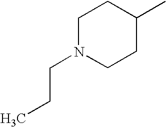 | NH | S | N | H | 0 | NH | H | Cl | F |
| | Synthetic Route: A Supporting Data: LCMS M/z(+) 421 (MH⁺) | | | | | | | | | |
| 2 | 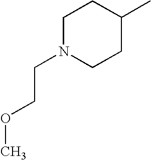 | NH | S | N | H | 0 | NH | H | Cl | F |
| | Synthetic Route: A Supporting Data: LCMS M/z(+) 437 (MH⁺) | | | | | | | | | |
| 3 | 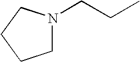 | NH | S | N | H | 0 | NH | H | Cl | F |
| | Synthetic Route: A Supporting Data: LCMS M/z(+) 393 (MH⁺) | | | | | | | | | |
| 4 | 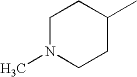 | NH | S | N | H | 0 | NH | H | Cl | F |
| | Synthetic Route: A Supporting Data: LCMS M/z(+) 393 (MH⁺) | | | | | | | | | |
| 5 | 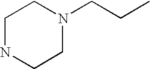 | NH | S | N | H | 0 | NH | H | Cl | F |
| | Synthetic Route: A Supporting Data: LCMS M/z(+) 407 (MH⁺) | | | | | | | | | |
| 6 | 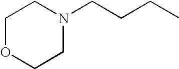 | NH | S | N | H | 0 | NH | H | Cl | F |
| | Synthetic Route: A Supporting Data: LCMS M/z(+) 423 (MH⁺) | | | | | | | | | |
| 7 | —(CH₂)₂N(CH₃)₂ | NH | S | N | H | 0 | NH | H | Cl | F |
| | Synthetic Route: A Supporting Data: LCMS M/z(+) 367 (MH⁺) | | | | | | | | | |
| 8 | —(CH₂)₂N(C₂H₅)₂ | NH | S | N | H | 0 | NH | H | Cl | F |
| | Synthetic Route: A Supporting Data: LCMS M/z(+) 395 (MH⁺) | | | | | | | | | |
| 9 | 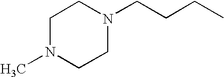 | NH | S | N | H | 0 | NH | H | Cl | F |
| | Synthetic Route: A Supporting Data: LCMS M/z(+) 436 (MH⁺) | | | | | | | | | |
| 10 | —CH₂C(CH₃)₂CH₂N(CH₃)₂ | NH | S | N | H | 0 | NH | H | Cl | F |
| | Synthetic Route: A Supporting Data: LCMS M/z(+) 409 (MH⁺) | | | | | | | | | |
| 11 | 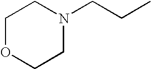 | NH | S | N | H | 0 | NH | H | Cl | F |
| | Synthetic Route: A Supporting Data: LCMS M/z(+) 409 (MH⁺) | | | | | | | | | |
| 12 | 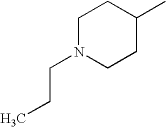 | HN(O)C | CH | S | H | 0 | NH | H | Cl | F |
| | Synthetic Route: E Supporting Data: LCMS M/z(+) 447.89 (MH⁺) | | | | | | | | | |
| 13 | 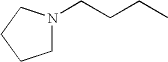 | HN(O)C | CH | S | H | 0 | NH | H | Cl | F |
| | Synthetic Route: E Supporting Data: LCMS M/z(+) 434 (MH⁺) | | | | | | | | | |
| 14 | —(CH₂)₃N(CH₃)₂ | (H₃C)N | CH | S | H | 0 | NH | H | Cl | F |
| | | (O)C— | | | | | | | | |
| | Synthetic Route: E Supporting Data: LCMS M/z(+) 422 (MH⁺) | | | | | | | | | |
| 15 | 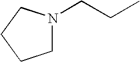 | HN(O)C— | CH | S | H | 0 | NH | H | Cl | F |
| | Synthetic Route: E Supporting Data: LCMS M/z(+) 419.96 (MH⁺) | | | | | | | | | |
| 16 | 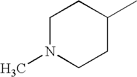 | —H₃C)N (O)— | CH | S | H | 0 | NH | H | Cl | F |
| | Synthetic Route: E Supporting Data: LCMS M/z(+) 434 (MH⁺) | | | | | | | | | |
| 17 | 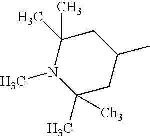 | HN(O)C— | CH | S | H | 0 | NH | H | Cl | F |
| | Synthetic Route: E Supporting Data: LCMS M/z(+) 476 (MH⁺) | | | | | | | | | |
| 18 | 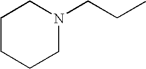 | HN(O)C— | CH | S | H | 0 | NH | H | Cl | F |
| | Synthetic Route: E Supporting Data: LCMS M/z(+) 434 (MH⁺) | | | | | | | | | |
| 19 | 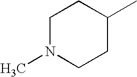 | HN(O)C— | CH | S | H | 0 | NH | H | —(CH₂)₃— | |
| | Synthetic Route: E Supporting Data: LCMS M/z(+) 408 (MH⁺) | | | | | | | | | |
| 20 | 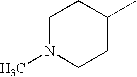 | HN(O)C— | CH | S | H | 0 | NH | H | —CH₂OCH₂— | |
| | Synthetic Route: E Supporting Data: LCMS M/z(+) 410 (MH⁺) | | | | | | | | | |
| 21 | 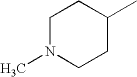 | HN(O)C— | CH | S | H | 0 | NH | H | —NHCH═CH— | |
| | Synthetic Route: E Supporting Data: LCMS M/z(+) 407 (MH⁺) | | | | | | | | | |
| 22 | 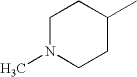 | HN(O)C— | CH | S | H | 0 | NH | H | F | Cl |
| | Synthetic Route: E Supporting Data: LCMS M/z(+) 420 (MH⁺) | | | | | | | | | |
| 23 | 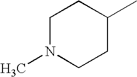 | HN(O)C— | CH | S | H | 0 | NH | H | F | F |
| | Synthetic Route: E Supporting Data: LCMS M/z(+) 404 (MH⁺) | | | | | | | | | |
| 24 | 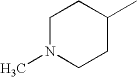 | HN(O)C— | CH | S | H | 0 | NH | H | H | F |
| | Synthetic Route: E Supporting Data: LCMS M/z(+) 386 (MH⁺) | | | | | | | | | |
| 25 | 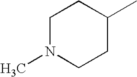 | HN(O)C— | CH | S | H | 0 | NH | H | —OCH₂O— | |
| | Synthetic Route: E Supporting Data: LCMS M/z(+) 412 (MH⁺) | | | | | | | | | |
| 26 | 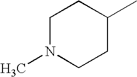 | HN(O)C— | CH | S | H | 0 | NH | H | H | H |
| | Synthetic Route: E Supporting Data: LCMS M/z(+) 368 (MH⁺) | | | | | | | | | |
| 27 | 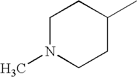 | HN(O)C— | CH | S | H | 0 | NH | H | Cl | H |
| | Synthetic Route: E Supporting Data: LCMS M/z(+) 402 (MH⁺) | | | | | | | | | |
| 28 | 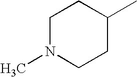 | HN(O)C— | CH | S | H | 0 | NH | H | H | Cl |
| | Synthetic Route: E Supporting Data: LCMS M/z(+) 403 (MH⁺) | | | | | | | | | |
| 29 | 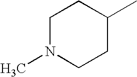 | HN(O)C— | CH | S | H | 1 | NH | H | H | H |
| | Synthetic Route: E Supporting Data: LCMS M/z(+) 382 (MH⁺) | | | | | | | | | |
| 30 | 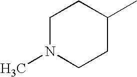 | HN(O)C— | CH | S | H | 0 | NH | H | —(CH₂)₂O— | |
| | Synthetic Route: E Supporting Data: LCMS M/z(+) 410 (MH⁺) | | | | | | | | | |
| 31 | 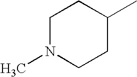 | HN(O)C— | CH | S | H | 0 | NH | H | OCH₃ | H |
| | Synthetic Route: E Supporting Data: LCMS M/z(+) 435 (MH⁺) | | | | | | | | | |
| 32 | 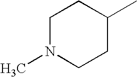 | HN(O)C— | CH | S | H | 0 | NH | H | Cl | OH |
| | Synthetic Route: E Supporting Data: LCMS M/z(+) 418 (MH⁺) | | | | | | | | | |
| 33 | 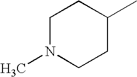 | HN(O)C— | CH | S | H | 0 | NH | H | —O(CH₂)₃O— | |
| | Synthetic Route: E Supporting Data: LCMS M/z(+) 440 (MH⁺) | | | | | | | | | |
| 34 | 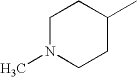 | HN(O)C— | CH | S | H | 0 | NH | H | CN | CH₃ |
| | Synthetic Route: E Supporting Data: LCMS M/z(+) 407 (MH⁺) | | | | | | | | | |
| 35 | 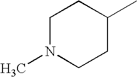 | HN(O)C— | CH | S | H | 0 | NH | H | CH₃ | F |
| | Synthetic Route: E Supporting Data: LCMS M/z(+) 400 (MH⁺) | | | | | | | | | |
| 36 | 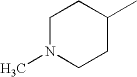 | HN(O)C— | CH | S | H | 0 | NH | H | 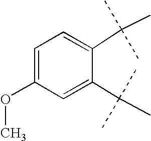 | |
| | Synthetic Route: E Supporting Data: LCMS M/z(+) 418 (MH⁺) | | | | | | | | | |
| 37 | 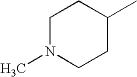 | HN(O)C— | CH | S | H | 0 | NH | H | —SCH₃ | H |
| | Synthetic Route: E Supporting Data: LCMS M/z(+) 414 (MH⁺) | | | | | | | | | |
| 38 | 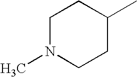 | HN(O)C— | CH | S | H | 0 | NH | H | —C(O) CH₃ | H |
| | Synthetic Route: E Supporting Data: LCMS M/z(+) 410 (MH⁺) | | | | | | | | | |
| 39 | 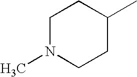 | HN(O)C— | CH | S | H | 0 | NH | H | —C≡CH | H |
| | Synthetic Route: E Supporting Data: LCMS M/z(+) 392 (MH⁺) | | | | | | | | | |
| 40 | 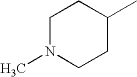 | HN(O)C— | CH | S | H | 0 | NH | H | —SCH═N— | |
| | Synthetic Route: E Supporting Data: LCMS M/z(+) 425 (MH⁺) | | | | | | | | | |
| 41 | 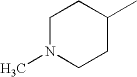 | HN(O)C— | S | C H | H | 0 | NH | H | Cl | F |
| | Synthetic Route: E Supporting Data: NMR δ(CD₃SOCD₃) 1.50-1.65(2h, m), | | | | | | | | | |
| | 1.74-1.85(2H, m), 1.89-1.99(2H, m), 2.16(3H, s), 2.72-2.82(2H, m), 3.64-3.80(1H, | | | | | | | | | |
| | m), 7.42(1H, t), 7.71-7.79(1H, m), 8.13-8.20(2H, m), 8.64(1H, s), 8.74(1H, d), 9.91 | | | | | | | | | |
| | (1H, s); LCMS M/z(−) 418 (M − H). | | | | | | | | | |
| 42 | 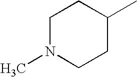 | HN(O)C— | CH | S | H | 1 | NH | H | Cl | F |
| | Synthetic Route: E Supporting Data: LCMS M/z(+) 434 (MH⁺) | | | | | | | | | |
| 43 | 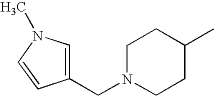 | HN(O)C— | CH | S | H | 0 | NH | H | Cl | F |
| | Synthetic Route: B, C Supporting Data: LCMS M/z(+) 499 (MH⁺) | | | | | | | | | |
| 44 | 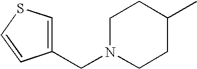 | HN(O)C— | CH | S | H | 0 | NH | H | Cl | F |
| | Synthetic Route: B, C Supporting Data: LCMS M/z(+) 502 (MH⁺) | | | | | | | | | |
| 45 | 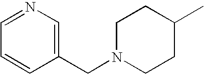 | HN(O)C— | CH | S | H | 0 | NH | H | Cl | F |
| | Synthetic Route: B, C Supporting Data: LCMS M/z(+) 497 (MH⁺) | | | | | | | | | |
| 46 | 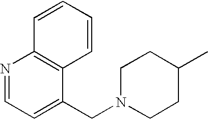 | HN(O)C— | CH | S | H | 0 | NH | H | Cl | F |
| | Synthetic Route: B, C Supporting Data: LCMS M/z(+) 547 (MH⁺) | | | | | | | | | |
| 47 | 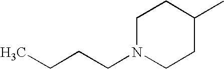 | HN(O)C— | CH | S | H | 0 | NH | H | Cl | F |
| | Synthetic Route: B, C Supporting Data: LCMS M/z(+) 462 (MH⁺) | | | | | | | | | |
| 48 | 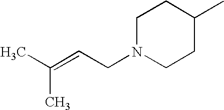 | HN(O)C— | CH | S | H | 0 | NH | H | Cl | F |
| | Synthetic Route: B, C Supporting Data: LCMS M/z(+) 474 (MH⁺) | | | | | | | | | |
| 49 | 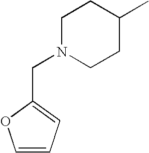 | HN(O)C— | CH | S | H | 0 | NH | H | Cl | F |
| | Synthetic Route: B, C Supporting Data: LCMS M/z(+) 486 (MH⁺) | | | | | | | | | |
| 50 | 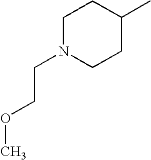 | HN(O)C— | CH | S | H | 0 | NH | H | Cl | F |
| | Synthetic Route: E Supporting Data: LCMS M/z(+) 464.01 (MH⁺) | | | | | | | | | |
| 51 | 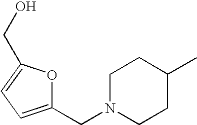 | HN(O)C— | CH | S | H | 0 | NH | H | Cl | F |
| | Synthetic Route: B, C Supporting Data: LCMS M/z(+) 516 (MH⁺) | | | | | | | | | |
| 52 | 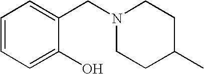 | HN(O)C— | CH | S | H | 0 | NH | H | Cl | F |
| | Synthetic Route: B, C Supporting Data: LCMS M/z(+) 512 (MH⁺) | | | | | | | | | |
| 53 | 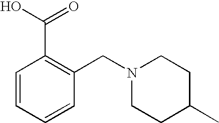 | HN(O)C— | CH | S | H | 0 | NH | H | Cl | F |
| | Synthetic Route: B, C Supporting Data: LCMS M/z(+) 540 (MH⁺) | | | | | | | | | |
| 54 | 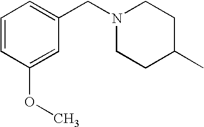 | HN(O)C— | CH | S | H | 0 | NH | H | Cl | F |
| | Synthetic Route: B, C Supporting Data: LCMS M/z(+) 526 (MH⁺) | | | | | | | | | |
| 55 | 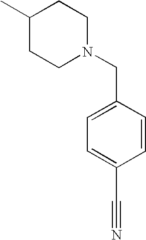 | HN(O)C— | CH | S | H | 0 | NH | H | Cl | F |
| | Synthetic Route: B, C Supporting Data: LCMS M/z(+) 521 (MH⁺) | | | | | | | | | |
| 56 | 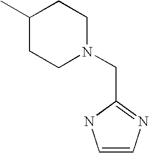 | HN(O)C— | CH | S | H | 0 | NH | H | Cl | F |
| | Synthetic Route: B, C Supporting Data: LCMS M/z(+) 486 (MH⁺) | | | | | | | | | |
| 57 | 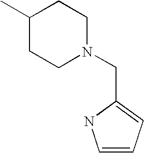 | HN(O)C— | CH | S | H | 0 | NH | H | Cl | F |
| | Synthetic Route: B, C Supporting Data: LCMS M/z(+) 484 (MH⁺) | | | | | | | | | |
| 58 | 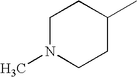 | HN(O)C— | CH | S | H | 0 | NC H₃ | H | Cl | H |
| | Synthetic Route: E Supporting Data: LCMS M/z(+) 416 (MH⁺) | | | | | | | | | |
| 59 | 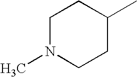 | HN(O)C— | CH | S | H | 0 | NC H₃ | H | H | Cl |
| | Synthetic Route: E Supporting Data: LCMS M/z(+) 416 (MH⁺) | | | | | | | | | |
| 60 | 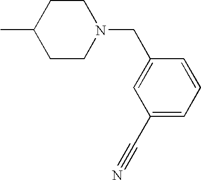 | HN(O)C— | CH | S | H | 0 | NH | H | Cl | F |
| | Synthetic Route: B, C Supporting Data: LCMS M/z(+) 521 (MH⁺) | | | | | | | | | |
| 61 | 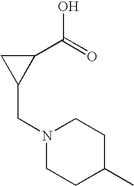 | HN(O)C— | CH | S | H | 0 | NH | H | Cl | F |
| | Synthetic Route: B, C Supporting Data: LCMS M/z(+) 531 (MH⁺) | | | | | | | | | |
| 62 | 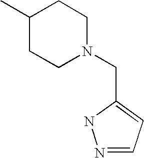 | HN(O)C— | CH | S | H | 0 | NH | H | Cl | F |
| | Synthetic Route: B, C Supporting Data: LCMS M/z(+) 486 (MH⁺) | | | | | | | | | |
| 63 | 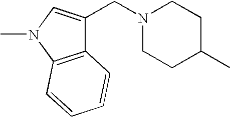 | HN(O)C— | CH | S | H | 0 | NH | H | Cl | F |
| | Synthetic Route: B, C Supporting Data: LCMS M/z(+) 549 (MH⁺) | | | | | | | | | |
| 64 | 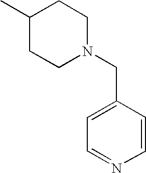 | HN(O)C— | CH | S | H | 0 | NH | H | Cl | F |
| | Synthetic Route: B, C Supporting Data: LCMS M/z(+) 497 (MH⁺) | | | | | | | | | |
| 65 | 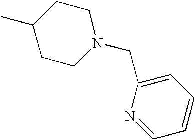 | HN(O)C— | CH | S | H | 0 | NH | H | Cl | F |
| | Synthetic Route: B, C Supporting Data: LCMS M/z(+) 497 (MH⁺) | | | | | | | | | |
| 66 | 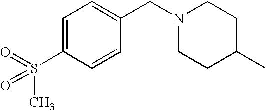 | HN(O)C— | CH | S | H | 0 | NH | H | Cl | F |
| | Synthetic Route: B, C Supporting Data: LCMS M/z(+) 574 (MH⁺) | | | | | | | | | |
| 67 | 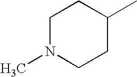 | HN(O)C— | CH | S | H | 0 | —N(CH₂)₂— | | H | H |
| | Synthetic Route: E Supporting Data: LCMS M/z(+) 394 (MH⁺) | | | | | | | | | |
| 68 | 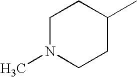 | HN(O)C— | CH | S | H | 0 | NH | H | —CH═CHNH— | |
| | Synthetic Route: E Supporting Data: LCMS M/z(+) 407 (MH⁺) | | | | | | | | | |
| 69 | 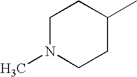 | HN(O)C— | CH | S | H | 0 | NH | H | Cl | Cl |
| | Synthetic Route: E Supporting Data: NMR δ(CD₃SOCD₃) 1.75(m, 2H), 1.95(m, | | | | | | | | | |
| | 2H), 2.60(m, 2H), 2.80(m, 2H), 3.95(m, 1H), 7.65(m, 1H), 7.85(m, 1H), 8.32(m, | | | | | | | | | |
| | 1H), 8.40(m, 1H), 8.65(m, 1H), 8.75(m, 2H), 10.10(s, 1H); LCMS M/z(+) 436 (MH⁺). | | | | | | | | | |
| 70 | 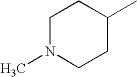 | HN(O)C— | CH | S | H | 0 | NH | H | CN | H |
| | Synthetic Route: E Supporting Data: NMR δ(CD₃SOCD₃) 1.60(m, 2H), 1.80(m, | | | | | | | | | |
| | 2H), 1.97(m, 2H), 2.18(s, 3H), 2.77(m, 2H), 3.70(m, 1H), 7.55(m, 2H), 8.07(m, | | | | | | | | | |
| | 1H), 8.40(m, 2H), 8.60(m, 2H), 10.14(s, 1H); LCMS M/z(+) 393 (MH⁺). | | | | | | | | | |
| 71 | 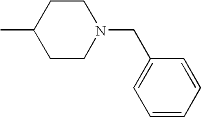 | HN(O)C— | CH | S | H | 0 | NH | H | Cl | F |
| | Synthetic Route: E Supporting Data: LCMS M/z(+) 496 (MH⁺) | | | | | | | | | |
| 72 | 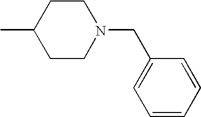 | HN(O)C— | CH | S | H | 0 | NH | H | H | F |
| | Synthetic Route: E Supporting Data: NMR δ(CD₃SOCD₃) 1.60(m, 2H), 1.80(m, | | | | | | | | | |
| | 2H), 2.04(m, 2H), 2.80(m, 2H), 3.43(m, 2H), 3.78(m, 1H), 7.14-7.40(m, 7H), 7.80 | | | | | | | | | |
| | (m, 2H), 8.30(s, 1H), 8.50(s, 1H), 8.55(m, 1H), 9.85(s, 1H); LCMS M/z(+) 462 (MH⁺) | | | | | | | | | |
| 73 | 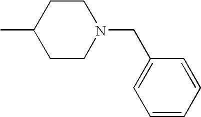 | HN(O)C— | CH | S | H | 0 | NH | H | H | H |
| | Synthetic Route: E Supporting Data: NMR δ(CD₃SOCD₃) 1.60(m, 2H), 1.80(m, | | | | | | | | | |
| | 2H), 2.04(m, 2H), 2.80(m, 2H), 3.43(m, 2H), 3.78(m, 1H), 7.05(m, 1H), 7.20-7.40 | | | | | | | | | |
| | (m, 7H), 7.80(m, 2H), 8.35(s, 1H), 8.52(s, 1H), 9.82(s, 1H); LCMS M/z(+) 444 (MH⁺) | | | | | | | | | |
| 74 | 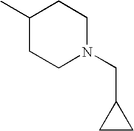 | HN(O)C— | CH | S | H | 0 | NH | H | Cl | F |
| | Synthetic Route: B, C Supporting Data: LCMS M/z(+) 460 (MH⁺) | | | | | | | | | |
| 75 | 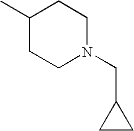 | HN(O)C— | CH | S | H | 0 | NH | H | H | F |
| | Synthetic Route: E Supporting Data: LCMS M/z(+) 426 (MH⁺) | | | | | | | | | |
| 76 | 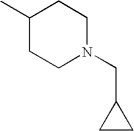 | HN(O)C— | CH | S | H | 0 | NH | H | F | F |
| | Synthetic Route: E Supporting Data: LCMS M/z(+) 444 (MH⁺) | | | | | | | | | |
| 77 | 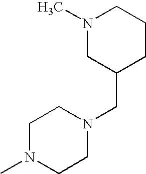 | —(O)C— | CH | S | H | 0 | NH | H | Cl | F |
| | Synthetic Route: E Supporting Data: NMR δ(CD₃SOCD₃) 1.50-2.10(m, 3H), | | | | | | | | | |
| | 2.25(d, 2H), 2.40(m, 2H), 2.50-2.65(m, 6H), 2.88(s, 3H), 3.40(m, 2H), 3.70(m, | | | | | | | | | |
| | 4H), 7.44(dd, 1H), 7.75(ddd, 1H), 8.10(s, 1H), 8.12(dd, 1H), 8.59(s, 1H), 9.84(s, 1H). | | | | | | | | | |
| 78 | 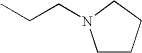 | HN(O)C— | CH | S | H | 0 | NH | H | Cl | F |
| | Synthetic Route: E Supporting Data: NMR δ(CD₃SOCD₃) 1.83(m, 4H), 2.98(t, | | | | | | | | | |
| | 2H), 3.09(m, 4H), 3.48(t, 2H), 7.36(s, 1H), 7.40(dd, 1H), 7.78(ddd, 1H), 8.16(dd, | | | | | | | | | |
| | 1H), 8.41(s, 1H), 9.63(s, 1H), 11.87(s, 1H), LCMS M/z(+) 420.3/422.3 (MH⁺) | | | | | | | | | |
| 79 | 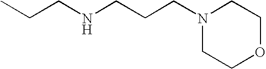 | HN(O)C— | CH | S | H | 0 | NH | H | Cl | F |
| | Synthetic Route: E Supporting Data: NMR δ(CD₃SOCD₃) 1.76(m, 2H), 2.30- | | | | | | | | | |
| | 2.45(m, 8H), 2.82(t, 2H), 3.27(t, 2H), 3.60(m, 4H), 7.33(s, 1H), 7.40(dd, 1H), 7.78 | | | | | | | | | |
| | (ddd, 1H), 8.07(dd, 1H), 8.43(s, 1H), 9.63(s, 1H), LCMS M/z(+) 493.3, 495.3 (MH⁺) | | | | | | | | | |
| 80 | 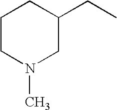 | —O— | S | N | H | 0 | NH | H | Cl | F |
| | Synthetic Route: A Supporting Data: NMR δ(CD₃SOCD₃, 373K) 1.2(m, 1H), 1.6 | | | | | | | | | |
| | (m, 1H), 1.7(m, 2H), 1.95(m, 1H), 2.05(m, 1H), 2.2(m, 4H), 2.6(m, 1H), 2.8(m, | | | | | | | | | |
| | 1H), 4.6(m, 2H), 7.3(t, 1H), 7.7(m, 1H), 8.0(dd, 1H), 8.6(s, 1H), 9.5(br s, 1H); | | | | | | | | | |
| | LCMS M/z(+) 408/410 (MH⁺) | | | | | | | | | |
| 81 | 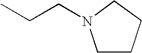 | —O— | S | N | H | 0 | NH | H | Cl | F |
| | Synthetic Route: A Supporting Data: NMR δ(CD₃SOCD₃) 1.7(m, 4H), 2.5 under | | | | | | | | | |
| | DMSO(m, ˜4H), 2.9(t, 2H), 4.7(t, 2H), 7.4(t, 1H), 7.6(m, 1H), 8.0(dd, 1H), 8.6(s, | | | | | | | | | |
| | 1H), 9.8(br s, 1H); LCMS M/z(+) 394/396 (MH⁺) | | | | | | | | | |
| 82 | 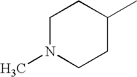 | HN(O)C— | CH | S | H | 0 | NH | H | Cl | F |
| | Synthetic Route: E Supporting Data: LCMS M/z(+) 419.89 (MH⁺) | | | | | | | | | |
| 83 | 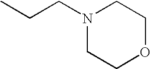 | —HN— | S | N | H | 0 | NH | H | H | F |
| | Synthetic Route: A Supporting Data: LCMS M/z(−) 374 (M − H) | | | | | | | | | |
| 84 | 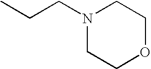 | —HN— | S | N | H | 0 | NH | H | H | Cl |
| | Synthetic Route: A Supporting Data: LCMS M/z(+) 390 (MH⁺) | | | | | | | | | |
| 85 | 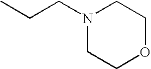 | —HN— | S | N | H | 0 | NH | H | —(CH₂)₃— | |
| | Synthetic Route: A Supporting Data: LCMS M/z(+) 396.94 (MH⁺) | | | | | | | | | |
| 86 | 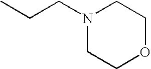 | —HN— | S | N | H | 0 | NH | H | Cl | Cl |
| | Synthetic Route: A Supporting Data: LCMS M/z(+) 424.79 (MH⁺) | | | | | | | | | |
| 87 | 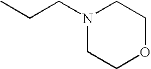 | HN(O)C— | CH | S | H | 0 | NH | H | Cl | F |
| | Synthetic Route: E Supporting Data: LCMS M/z(+) 436.04 (MH⁺) | | | | | | | | | |
| 88 | 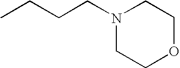 | HN(O)C— | CH | S | H | 0 | NH | H | Cl | F |
| | Synthetic Route: E Supporting Data: LCMS M/z(+) 449.99 (MH⁺) | | | | | | | | | |
| 89 | —(CH₂)₂N(C₂H₅)₂ | HN(O)C— | CH | S | H | 0 | NH | H | Cl | F |
| | Synthetic Route: E Supporting Data: LCMS M/z(+) 422.04 (MH⁺) | | | | | | | | | |
| 90 | —(CH₂)₃N(CH₃)₂ | HN(O)C— | CH | S | H | 0 | NH | H | Cl | F |
| | Synthetic Route: E Supporting Data: LCMS M/z(+) 422.04 (MH⁺) | | | | | | | | | |
| 91 | —CH₂C(CH₃)₂CH₂ | HN(O)C— | CH | S | H | 0 | NH | H | Cl | F |
| | N(CH₃)₂ | | | | | | | | | |
| | Synthetic Route: E Supporting Data: LCMS M/z(+) 436 (MH⁺) | | | | | | | | | |
| 92 | 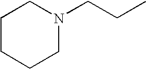 | —HN— | S | N | H | 0 | NH | H | H | H |
| | Synthetic Route: A Supporting Data: LCMS M/z(+) 355.05 (MH⁺) | | | | | | | | | |
| 93 | 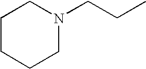 | —HN— | S | N | H | 0 | NH | H | Cl | H |
| | Synthetic Route: A Supporting Data: LCMS M/z(+) 388.91 (MH⁺) | | | | | | | | | |
| 94 | 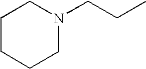 | —HN— | S | N | H | 0 | NH | H | CH₃ | H |
| | Synthetic Route: A Supporting Data: LCMS M/z(+) 369.06 (MH⁺) | | | | | | | | | |
| 95 | 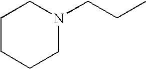 | —HN— | S | N | H | 0 | NH | H | H | F |
| | Synthetic Route: A Supporting Data: LCMS M/z(+) 373 (MH⁺) | | | | | | | | | |
| 96 | 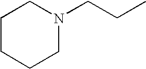 | —HN— | S | N | H | 0 | NH | H | H | Cl |
| | Synthetic Route: A Supporting Data: LCMS M/z(+) 389 (MH⁺) | | | | | | | | | |
| 97 | 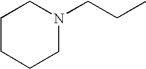 | —HN— | S | N | H | 0 | NH | H | H | CH₃ |
| | Synthetic Route: A Supporting Data: LCMS M/z(+) 369.02 (MH⁺) | | | | | | | | | |
| 98 | 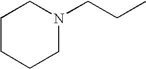 | —HN— | S | N | H | 0 | NH | H | F | F |
| | Synthetic Route: A Supporting Data: LCMS M/z(+) 391.09 (MH⁺) | | | | | | | | | |
| 99 | 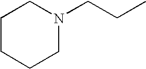 | —HN— | S | N | H | 0 | O | H | Cl | F |
| | Synthetic Route: A Supporting Data: LCMS M/z(+) 409.9 (MH⁺) | | | | | | | | | |
| 100 | 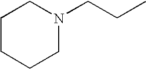 | —HN— | S | N | H | 0 | NH | H | —(CH₂)₃— | |
| | Synthetic Route: A Supporting Data: LCMS M/z(+) 395.1 (MH⁺) | | | | | | | | | |
| 101 | 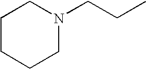 | —HN— | S | N | H | 0 | NH | H | Cl | F |
| | Synthetic Route: A Supporting Data: Supporting Data: NMR δ(CD₃SOCD₃) 1.4 | | | | | | | | | |
| | (m, 2H), 1.5(m, 4H), 2.4(m, 2H), 2.5 under DMSO peak (m, ?H), 3.5(m, 2H), 7.4(t, | | | | | | | | | |
| | 1H), 7.6(m, 1H), 8.0(dd, 1H), 8.4(s, 1H), 8.7(m, 1H), 9.3(s, 1H; LCMS M/z(+) 407/409 (MH⁺) | | | | | | | | | |
| 102 | —(CH₂)₂N(CH₃)₂ | HN(O)C— | CH | S | H | 0 | NH | H | Cl | F |
| | Synthetic Route: E Supporting Data: Retention time LCMS M/z(+) 393.91 (MH⁺) | | | | | | | | | |
| 104 | 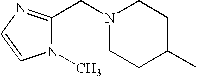 | HN(O)C— | CH | S | H | 0 | NH | H | Cl | F |
| | Synthetic Route: B, C Supporting Data: LCMS M/z(+) 499.92 (MH⁺) | | | | | | | | | |
| 105 | 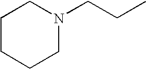 | —HN— | S | N | CH₃ | 0 | NH | H | Cl | F |
| | Synthetic Route: A (acetyl chloride modification) Supporting Data: LCMS | | | | | | | | | |
| | M/z(+) 420.87 (MH⁺) | | | | | | | | | |
| 106 | 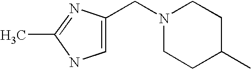 | HN(O)C— | CH | S | H | 0 | NH | H | Cl | F |
| | Synthetic Route: B, C Supporting Data: LCMS M/z(+) 499.85 (MH⁺) | | | | | | | | | |
| 107 | 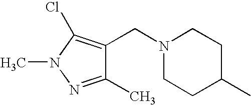 | HN(O)C— | CH | S | H | 0 | NH | H | Cl | F |
| | Synthetic Route: B, C Supporting Data: LCMS M/z(+) 547.84 (MH⁺) | | | | | | | | | |
| 108 | 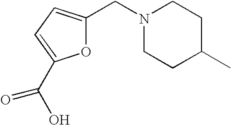 | HN(O)C— | CH | S | H | 0 | NH | H | Cl | F |
| | Synthetic Route: B, C Supporting Data: LCMS M/z(+) 529.83 (MH⁺) | | | | | | | | | |
| 109 | 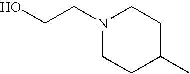 | HN(O)C— | CH | S | H | 0 | NH | H | Cl | F |
| | Synthetic Route: B, C Supporting Data: LCMS M/z(+) 449.83 (MH⁺) | | | | | | | | | |
| 110 | 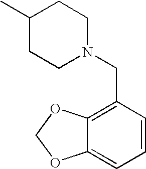 | HN(O)C— | CH | S | H | 0 | NH | H | Cl | F |
| | Synthetic Route: B, C Supporting Data: LCMS M/z(+) 539.87 (MH⁺) | | | | | | | | | |
| 111 | 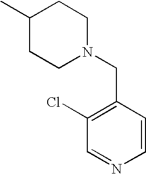 | HN(O)C— | CH | S | H | 0 | NH | H | Cl | F |
| | Synthetic Route: B, C Supporting Data: LCMS M/z(+) 530.84 (MH⁺) | | | | | | | | | |
| 112 | 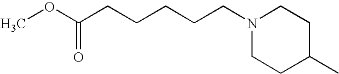 | HN(O)C— | CH | S | H | 0 | NH | H | Cl | F |
| | Synthetic Route: B, C Supporting Data: LCMS M/z(+) 533.96 (MH⁺) | | | | | | | | | |
| 113 | 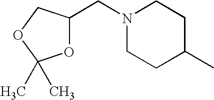 | HN(O)C— | CH | S | H | 0 | NH | H | Cl | F |
| | Synthetic Route: B, C Supporting Data: LCMS M/z(+) 519.99 (MH⁺) | | | | | | | | | |
| 114 | 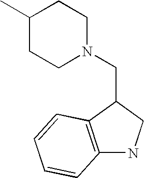 | HN(O)C— | CH | S | H | 0 | NH | H | Cl | F |
| | Synthetic Route: B, C Supporting Data: LCMS M/z(+) 534.9 (MH⁺) | | | | | | | | | |
| 115 | 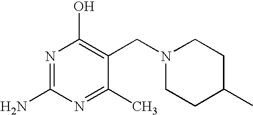 | HN(O)C— | CH | S | H | 0 | NH | H | Cl | F |
| | Synthetic Route: B, C Supporting Data: LCMS M/z(+) 542.88 (MH⁺) | | | | | | | | | |
| 116 | 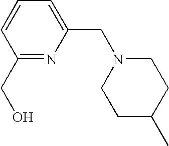 | HN(O)C— | CH | S | H | 0 | NH | H | Cl | F |
| | Synthetic Route: B, C Supporting Data: LCMS M/z(+) 526.92 (MH⁺) | | | | | | | | | |
| 117 | 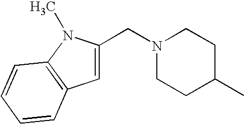 | HN(O)C— | CH | S | H | 0 | NH | H | Cl | F |
| | Synthetic Route: B, C Supporting Data: LCMS M/z(+) 548.96 (MH⁺) | | | | | | | | | |
| 118 | 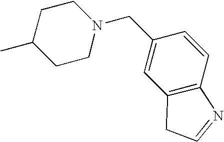 | HN(O)C— | CH | S | H | 0 | NH | H | Cl | F |
| | Synthetic Route: B, C Supporting Data: LCMS M/z(+) 534.97 (MH⁺) | | | | | | | | | |
| 119 | 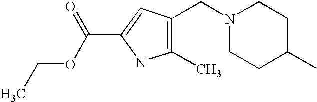 | HN(O)C— | CH | S | H | 0 | NH | H | Cl | F |
| | Synthetic Route: B, C Supporting Data: LCMS M/z(+) 571 (MH⁺) | | | | | | | | | |
| 120 | 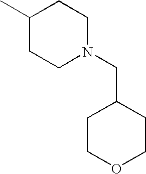 | HN(O)C— | CH | S | H | 0 | NH | H | Cl | F |
| | Synthetic Route: B, C Supporting Data: LCMS M/z(+) 503.98 (MH⁺) | | | | | | | | | |
| 121 | 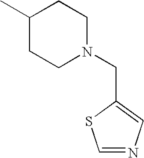 | HN(O)C— | CH | S | H | 0 | NH | H | Cl | F |
| | Synthetic Route: B, C Supporting Data: LCMS M/z(+) 502.91 (MH⁺) | | | | | | | | | |
| 122 | 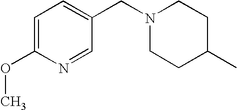 | HN(O)C— | CH | S | H | 0 | NH | H | Cl | F |
| | Synthetic Route: B, C Supporting Data: LCMS M/z(+) 526.95 (MH⁺) | | | | | | | | | |
| 123 | 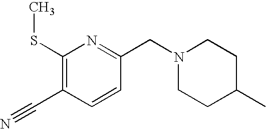 | HN(O)C— | CH | S | H | 0 | NH | H | Cl | F |
| | Synthetic Route: B, C Supporting Data: LCMS M/z(+) 567.92 (MH⁺) | | | | | | | | | |
| 124 | 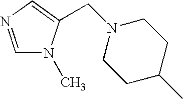 | HN(O)C— | CH | S | H | 0 | NH | H | Cl | F |
| | Synthetic Route: B, C Supporting Data: LCMS M/z(+) 499.92 (MH⁺) | | | | | | | | | |
| 125 | 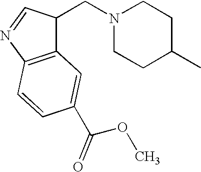 | HN(O)C— | CH | S | H | 0 | NH | H | Cl | F |
| | Synthetic Route: B, C Supporting Data: LCMS M/z(+) 592.98 (MH⁺) | | | | | | | | | |
| 126 | 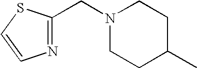 | HN(O)C— | CH | S | H | 0 | NH | H | Cl | F |
| | Synthetic Route: B, C Supporting Data: LCMS M/z(+) 503 (MH⁺) | | | | | | | | | |
| 127 | 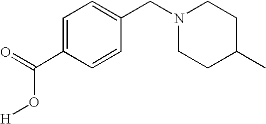 | HN(O)C— | CH | S | H | 0 | NH | H | Cl | F |
| | Synthetic Route: B, C Supporting Data: LCMS M/z(+) 540 (MH⁺) | | | | | | | | | |
| 128 | 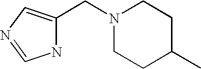 | HN(O)C— | CH | S | H | 0 | NH | H | Cl | F |
| | Synthetic Route: B, C Supporting Data: LCMS M/z(+) 486 (MH⁺) | | | | | | | | | |
| 129 | 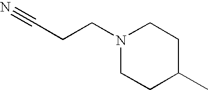 | HN(O)C— | CH | S | H | 0 | NH | H | Cl | F |
| | Synthetic Route: B, D Supporting Data: LCMS M/z(+) 459 (MH⁺) | | | | | | | | | |
| 130 | 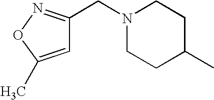 | HN(O)C— | CH | S | H | 0 | NH | H | Cl | F |
| | Synthetic Route: B, D Supporting Data: LCMS M/z(+) 501 (MH⁺) | | | | | | | | | |
| 131 | 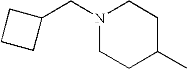 | HN(O)C— | CH | S | H | 0 | NH | H | Cl | F |
| | Synthetic Route: B, D Supporting Data: LCMS M/z(+) 474 (MH⁺) | | | | | | | | | |
| 132 | 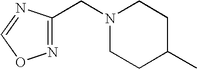 | HN(O)C— | CH | S | H | 0 | NH | H | Cl | F |
| | Synthetic Route: B, D Supporting Data: LCMS M/z(+) 488 (MH⁺) | | | | | | | | | |
| 133 | 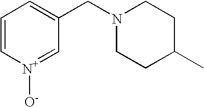 | HN(O)C— | CH | S | H | 0 | NH | H | Cl | F |
| | Synthetic Route: B, D Supporting Data: LCMS M/z(+) 513 (MH⁺) | | | | | | | | | |
| 134 | 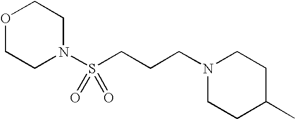 | HN(O)C— | CH | S | H | 0 | NH | H | Cl | F |
| | Synthetic Route: B, D Supporting Data: LCMS M/z(+) 597 (MH⁺) | | | | | | | | | |
| 135 | 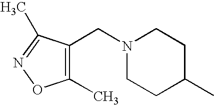 | HN(O)C— | CH | S | H | 0 | NH | H | Cl | F |
| | Synthetic Route: B, D Supporting Data: LCMS M/z(+) 515 (MH⁺) | | | | | | | | | |
| 136 | 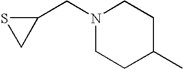 | HN(O)C— | CH | S | H | 0 | NH | H | Cl | F |
| | Synthetic Route: B, D Supporting Data: LCMS M/z(+) 477 (MH⁺) | | | | | | | | | |
| 137 | 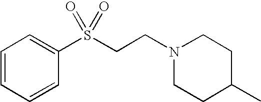 | HN(O)C— | CH | S | H | 0 | NH | H | Cl | F |
| | Synthetic Route: B, D Supporting Data: LCMS M/z(+) 574 (MH⁺) | | | | | | | | | |
| 138 | 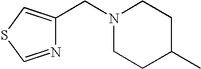 | HN(O)C— | CH | S | H | 0 | NH | H | Cl | F |
| | Synthetic Route: B, D Supporting Data: LCMS M/z(+) 503 (MH⁺) | | | | | | | | | |
| 139 | 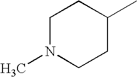 | HN(O)C— | CH | S | H | 1 | NH | H | H | OC H₃ |
| | Synthetic Route: E Supporting Data: LCMS M/z(+) 412 (MH⁺) | | | | | | | | | |
| 140 | 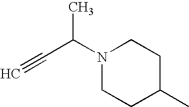 | HN(O)C— | CH | S | H | 0 | NH | H | Cl | F |
| | Synthetic Route: B, D Supporting Data: LCMS M/z(+) 458 (MH⁺) | | | | | | | | | |
| 141 | 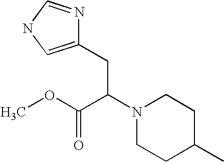 | HN(O)C— | CH | S | H | 0 | NH | H | Cl | F |
| | Synthetic Route: B, D Supporting Data: LCMS M/z(+) 558 (MH⁺) | | | | | | | | | |
| 142 | 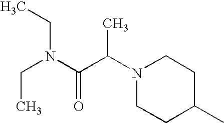 | HN(O)C— | CH | S | H | 0 | NH | H | Cl | F |
| | Synthetic Route: B, D Supporting Data: LCMS M/z(+) 533 (MH⁺) | | | | | | | | | |
| 143 | 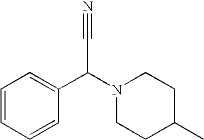 | HN(O)C— | CH | S | H | 0 | NH | H | Cl | F |
| | Synthetic Route: B, D Supporting Data: LCMS M/z(+) 521 (MH⁺) | | | | | | | | | |
| 144 | 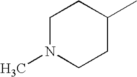 | HN(O)C— | CH | S | H | 0 | —NHC (O)— | H | H | H |
| | Synthetic Route: G Supporting Data: LCMS M/z(+) 396 (MH⁺) | | | | | | | | | |
| 145 | 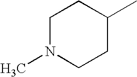 | HN(O)C— | CH | S | H | 0 | —NHC (O)— | H | F | F |
| | Synthetic Route: G Supporting Data: LCMS M/z(+) 432 (MH⁺) | | | | | | | | | |
| 146 | 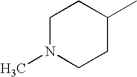 | HN(O)C— | CH | S | H | 0 | —NHC (O)— | H | H | Cl |
| | Synthetic Route: G Supporting Data: LCMS M/z(+) 430 (MH⁺) | | | | | | | | | |
| 147 | 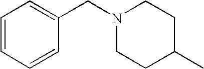 | HN(O)C— | CH | S | H | 0 | NH | H | H | CN |
| | Synthetic Route: E Supporting Data: LCMS M/z(+) 469 (MH⁺) | | | | | | | | | |
| 148 | 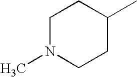 | HN(O)C— | CH | S | H | 0 | NH | H | CN | F |
| | Synthetic Route: E Supporting Data: NMR δ(CD₃SOCD₃) 1.60(m, 2H), 1.80 | | | | | | | | | |
| | (m, 2H), 1.97(m, 2H), 2.18(s, 3H), 2.77(m, 2H), 3.70(m, 1H), 7.55(t, 2H), 8.10(m, | | | | | | | | | |
| | 1H), 8.30(s, 1H), 8.41(m, 1H), 8.60(m, 2H), 10.13(s, 1H); LCMS M/z(+) 411 (MH⁺) | | | | | | | | | |
| 149 | 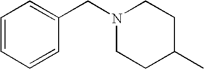 | HN(O)C— | CH | S | H | 0 | NH | H | F | F |
| | Synthetic Route: E Supporting Data: Supporting Data: NMR δ(CD₃SOCD₃) 1.60 | | | | | | | | | |
| | (m, 2H), 1.80(m, 2H), 2.02(m, 2H), 2.82(m, 2H), 3.48(s, 2H), 3.78(m, 1H), 7.20- | | | | | | | | | |
| | 7.35(m, 5H), 7.45(m, 1H), 7.60(m, 1H), 8.10(m, 1H), 8.35(s, 1H), 8.60(m, 2H), | | | | | | | | | |
| | 10.00(s, 1H); LCMS M/z(+) 480 (MH⁺) | | | | | | | | | |
| 150 | 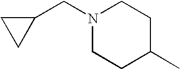 | HN(O)C— | CH | S | H | 0 | NH | H | H | H |
| | Synthetic Route: B, C Supporting Data: LCMS M/z(+) 408.5 (MH⁺) | | | | | | | | | |
| 151 | 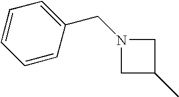 | HN(O)C— | CH | S | H | 0 | NH | H | F | F |
| | Synthetic Route: E, F, C Supporting Data: NMR δ(CD₃SOCD₃) 3.00-3.65 | | | | | | | | | |
| | (m, 6H), 4.45(m, 1H), 7.22(m, 5H), 7.40(m, 1H), 7.58(m, 1H), 8.10(m, 1H), 8.40 | | | | | | | | | |
| | (s, 1H), 8.60(s, 1H), 9.15(d, 1H), 10.05(s, 1H); LCMS M/z(+) 452.5 (MH⁺) | | | | | | | | | |
| 152 | 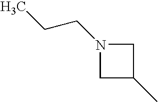 | HN(O)C— | CH | S | H | 0 | NH | H | F | F |
| | Synthetic Route: E, F, C Supporting Data: LCMS M/z(+) 404.4 (MH⁺) | | | | | | | | | |
| 153 | 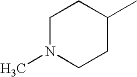 | HN(O)C— | CH | S | H | 0 | NH | H | 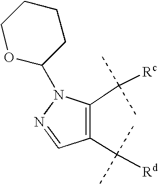 | |
| | Synthetic Route: E Supporting Data: LCMS M/z(−) 490.5 (MH⁻) | | | | | | | | | |
| 154 | 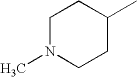 | HN(O)C— | CH | S | H | 0 | NH | H | 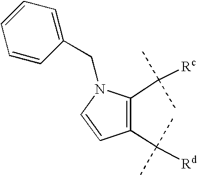 | |
| | Synthetic Route: E Supporting Data: LCMS M/z(+) 495.5 (MH⁺) | | | | | | | | | |
| 155 | 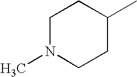 | HN(O)C— | CH | S | H | 0 | NH | H | 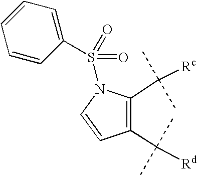 | |
| | Synthetic Route: E Supporting Data: LCMS M/z(+) 547 (MH⁺) | | | | | | | | | |
| 156 | 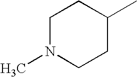 | HN(O)C— | CH | S | H | 0 | NH | H | —NHCH═N— | |
| | Synthetic Route: E Supporting Data: LCMS M/z(−) 406 (MH⁻) | | | | | | | | | |
| 157 | 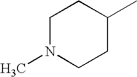 | HN(O)C— | CH | S | H | 0 | NH | H | —NHN═CH— | |
| | Synthetic Route: E Supporting Data: LCMS M/z(+) 408 (MH⁺) | | | | | | | | | |
| 158 | 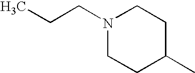 | HN(O)C— | CH | S | H | 0 | NH | H | —CH═CHNH— | |
| | Synthetic Route: E Supporting Data: NMR δ(CD₃SOCD₃) 0.82(t, | | | | | | | | | |
| | 3H), 1.40(m, 2H), 1.60(m, 2H), 1.80(m, 2H), 1.95(m, 2H), 2.22(m, 2H), 2.82(m, | | | | | | | | | |
| | 2H), 3.70(m, 1H), 6.40(s, 1H), 7.35(m, 3H), 7.90(s, 1H), 8.30(s, 1H), 8.42(s, 1H), | | | | | | | | | |
| | 8.45(d, 1H), 9.70(s, 1H), 11.02(s, 1H); LCMS M/z(−) 433 (MH⁻) | | | | | | | | | |
| 159 | 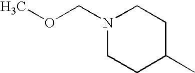 | HN(O)C— | CH | S | H | 0 | NH | H | —CH═CHNH— | |
| | Synthetic Route: E Supporting Data: NMR δ(CD₃SOCD₃) 1.58(m, 2H), 1.80(m, | | | | | | | | | |
| | 2H), 2.05(m, 2H), 2.42(m, 2H), 2.93(m, 2H), 3.20(s, 3H), 3.40(t, 2H), 3.70(m, | | | | | | | | | |
| | 1H), 6.40(s, 1H), 7.35(m, 3H), 7.93(s, 1H), 8.30(s, 1H), 8.40(s, 1H), 8.45(d, 1H), | | | | | | | | | |
| | 9.75(s, 1H), 11.02(s, 1H); LCMS M/z(−) 449 (MH⁻) | | | | | | | | | |
| 160 | 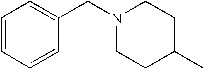 | HN(O)C— | CH | S | H | 0 | NH | H | —CH═CHNH— | |
| | Synthetic Route: E Supporting Data: NMR δ(CD₃SOCD₃) 1.60(m, 2H), 1.80(m, | | | | | | | | | |
| | 2H), 2.01(m, 2H), 2.80(m, 2H), 3.42(s, 2H), 3.75(m, 1H), 6.40(s, 1H), 7.30(m, | | | | | | | | | |
| | 8H), 7.95(s, 1H), 8.30(s, 1H), 8.40(s, 1H), 8.45(d, 1H), 9.75(s, 1H), 11.02(s, 1H); | | | | | | | | | |
| | LCMS M/z(+) 483 (MH⁺) | | | | | | | | | |
| 161 | 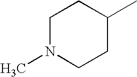 | HN(O)C— | CH | S | H | 0 | O | H | F | F |
| | Synthetic Route: E Supporting Data: LCMS M/z(+) 405 (MH⁺) | | | | | | | | | |
| 162 | 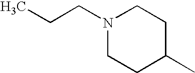 | HN(O)C— | CH | S | H | 0 | NH | H | —NHCH═CH— | |
| | Synthetic Route: E Supporting Data: LCMS M/z(−) 433 (M − H) | | | | | | | | | |
| 163 | 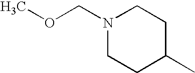 | HN(O)C— | CH | S | H | 0 | NH | H | —NHCH═CH— | |
| | Synthetic Route: E Supporting Data: LCMS M/z(−) 449 (M − H) | | | | | | | | | |
| 164 | 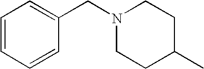 | HN(O)C— | CH | S | H | 0 | NH | H | —NHCH═CH— | |
| | Synthetic Route: E Supporting Data: LCMS M/z(−) 481 (M − H) | | | | | | | | | |
| 165 | 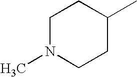 | HN(O)C— | N | S | H | 0 | NH | H | Cl | F |
| | Synthetic Route: H Supporting Data: LCMS M/z(+) 421 (MH⁺) | | | | | | | | | |
| 166 | 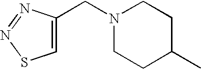 | HN(O)C— | CH | S | H | 0 | NH | H | Cl | F |
| | Synthetic Route: B, C Supporting Data: NMR δ(CD₃SOCD₃) 1.52-1.67(2H, m), | | | | | | | | | |
| | 1.75-1.86(2H, m), 2.15(2H, t), 2.84-2.94(2H, m), 3.67-3.81(1H, m), 4.02(2H, s), | | | | | | | | | |
| | 7.43(1H, t), 7.72-7.80(1H, m), 8.15-8.21(1H, m), 8.32(1H, s), 8.57-8.63(2H, m), | | | | | | | | | |
| | 9.02(1H, s), 10.007(1H, bs); LCMS M/z(+) 526 (MH⁺) | | | | | | | | | |
| 167 | 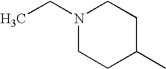 | HN(O)C— | CH | S | H | 0 | NH | H | Cl | F |
| | Synthetic Route: B, C Supporting Data: LCMS M/z(+) 432/434 (MH⁺) | | | | | | | | | |
| 168 | 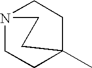 | HN(O)C— | CH | S | H | 0 | NH | H | Cl | F |
| | Synthetic Route: E Supporting Data: LCMS M/z(+) 430/432 (MH⁺) | | | | | | | | | |
| 169 | 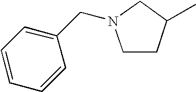 | HN(O)C— | CH | S | H | 0 | NH | H | Cl | F |
| | Synthetic Route: E Supporting Data: LCMS M/z(+) 482 (MH⁺) | | | | | | | | | |
| 170 | 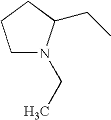 | HN(O)C— | CH | S | H | 0 | NH | H | Cl | F |
| | Synthetic Route: E Supporting Data: LCMS M/z(+) 434.79 (MH⁺) | | | | | | | | | |
| 171 | 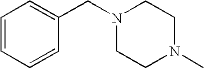 | HN(O)C— | CH | S | H | 0 | NH | H | Cl | F |
| | Synthetic Route: E Supporting Data: LCMS M/z(+) 482.79 (MH⁺) | | | | | | | | | |
| 172 | 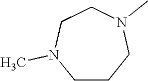 | HN(O)C— | CH | S | H | 0 | NH | H | Cl | F |
| | Synthetic Route: E Supporting Data: LCMS M/z(+) 420.78 (MH⁺) | | | | | | | | | |
| 173 | 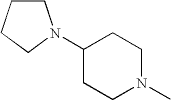 | HN(O)C— | CH | S | H | 0 | NH | H | Cl | F |
| | Synthetic Route: E Supporting Data: LCMS M/z(+) 460.81 (MH⁺) | | | | | | | | | |
| 174 | 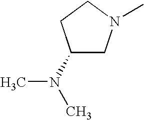 | HN(O)C— | CH | S | H | 0 | NH | H | Cl | F |
| | Synthetic Route: E Supporting Data: LCMS M/z(+) 420.77 (MH⁺) | | | | | | | | | |
| 175 | 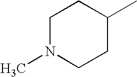 | HN(O)C— | S | C H | H | 0 | NH | H | NHCH═CH | |
| | Synthetic Route: E Supporting Data: NMR δ(CD₃SOCD₃) 1.48-1.65(2H, m), | | | | | | | | | |
| | 1.73-1.83(2H, m), 1.87-1.98(2H, m), 2.71-2.81(2H, m), 3.61-3.76(1H, m), 6.39-6.41 | | | | | | | | | |
| | (1H, m), 7.19-7.23(1H, m), 7.50(1H, d), 7.87(1H, s), 8.08(1H, s), 8.54(1H, s), 8.68 | | | | | | | | | |
| | (1H, d), 9.71(1H, s), 11.09(1H, bs); LCMS M/z(−) 405 (M − H) | | | | | | | | | |
| 176 | 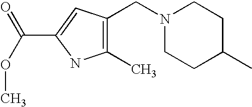 | HN(O)C— | CH | S | H | 0 | NH | H | Cl | F |
| | Synthetic Route: B, C Supporting Data: LCMS M/z(+) 552.95 (MH⁺) | | | | | | | | | |
| 177 | 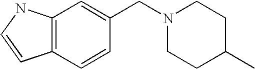 | HN(O)C— | CH | S | H | 0 | NH | H | Cl | F |
| | Synthetic Route: B, C Supporting Data: LCMS M/z(+) 534.97 (MH⁺) | | | | | | | | | |
| 178 | 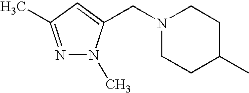 | HN(O)C— | CH | S | H | 0 | NH | H | Cl | F |
| | Synthetic Route: B, C Supporting Data: LCMS M/z(+) 513.98 (MH⁺) | | | | | | | | | |
| 179 | 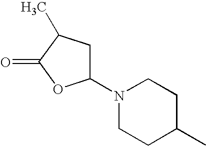 | HN(O)C— | CH | S | H | 0 | NH | H | Cl | F |
| | Synthetic Route: B, D Supporting Data: LCMS M/z(+) 503.98 (MH⁺) | | | | | | | | | |
| 180 | 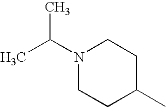 | HN(O)C— | CH | S | H | 0 | NH | H | Cl | F |
| | Synthetic Route: B, D Supporting Data: LCMS M/z(+) 448.01 (MH⁺) | | | | | | | | | |
| 181 | 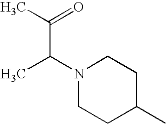 | HN(O)C— | CH | S | H | 0 | NH | H | Cl | F |
| | Synthetic Route: B, D Supporting Data: LCMS M/z(+) 475.99 (MH⁺) | | | | | | | | | |
| 182 | 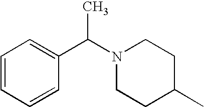 | HN(O)C— | CH | S | H | 0 | NH | H | Cl | F |
| | Synthetic Route: B, D Supporting Data: LCMS M/z(+) 509.99 (MH⁺) | | | | | | | | | |
| 183 | 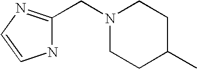 | HN(O)C— | CH | S | H | 0 | NH | H | Cl | F |
| | Synthetic Route: B, C Supporting Data: LCMS M/z(+) 459.31 (MH⁺) | | | | | | | | | |
| 184 | 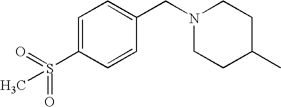 | HN(O)C— | CH | S | H | 0 | NH | H | CN | H |
| | Synthetic Route: B, C Supporting Data: LCMS M/z(+) 547.37 (MH⁺) | | | | | | | | | |
| 185 | 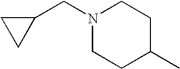 | HN(O)C— | CH | S | H | 0 | NH | H | CN | H |
| | Synthetic Route: B, C Supporting Data: LCMS M/z(+) 433.41 (MH⁺) | | | | | | | | | |
| 186 | 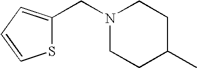 | HN(O)C— | CH | S | H | 0 | NH | H | CN | H |
| | Synthetic Route: B, C Supporting Data: LCMS M/z(+) 475.34 (MH⁺) | | | | | | | | | |
| 187 | 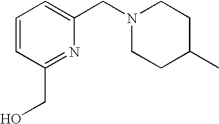 | HN(O)C— | CH | S | H | 0 | NH | H | CN | H |
| | Synthetic Route: B, C Supporting Data: LCMS M/z(+) 500.01 (MH⁺) | | | | | | | | | |
| 188 | 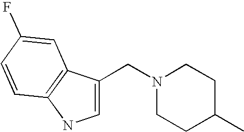 | HN(O)C— | CH | S | H | 0 | NH | H | CN | H |
| | Synthetic Route: B, C Supporting Data: LCMS M/z(+) 526.42 (MH⁺) | | | | | | | | | |
| 189 | 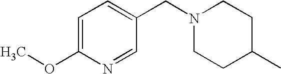 | HN(O)C— | CH | S | H | 0 | NH | H | CN | H |
| | Synthetic Route: B, C Supporting Data: LCMS M/z(+) 500.43 (MH⁺) | | | | | | | | | |
| 190 | 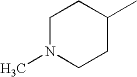 | HN(O)C— | CH | S | H | 0 | NH | H | CN | H |
| | Synthetic Route: E Supporting Data: LCMS M/z(+) 419 (MH⁺) | | | | | | | | | |
| 191 | 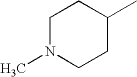 | HN(O)C— | CH | S | H | 0 | NH | H | 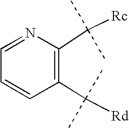 | |
| | Synthetic Route: E Supporting Data: LCMS M/z(−) 424 (M − H) | | | | | | | | | |
| 192 | 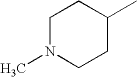 | HN(O)C— | CH | S | H | 0 | NH | H | —OCH₂CH₂O— | |
| | Synthetic Route: E Supporting Data: LCMS M/z(−) 437 (M − H) | | | | | | | | | |
| 193 | 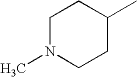 | HN(O)C— | CH | S | H | 0 | NH | H | 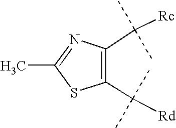 | |
| | Synthetic Route: E Supporting Data: LCMS M/z(−) 419 (M − H) | | | | | | | | | |
| 194 | 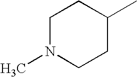 | HN(O)C— | CH | S | H | 0 | NH | H | 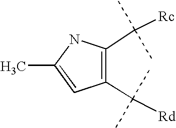 | |
| | Synthetic Route: E Supporting Data: LCMS M/z(+) 433 (MH⁺) | | | | | | | | | |
| 195 | 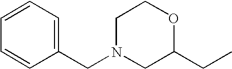 | HN(O)C— | CH | S | H | 0 | NH | H | 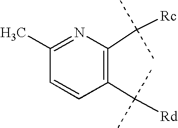 | |
| | Synthetic Route: E Supporting Data: LCMS M/z(+) 512 (MH⁺) | | | | | | | | | |
| 196 | 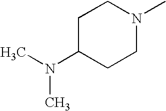 | HN(O)C— | CH | S | H | 0 | NH | H | Cl | F |
| | Synthetic Route: E Supporting Data: LCMS M/z(+) 434 (MH⁺) | | | | | | | | | |
| 197 | 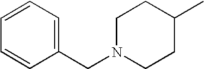 | —(O)C— | CH | S | H | 0 | NH | H | Cl | F |
| | Synthetic Route: E Supporting Data: LCMS M/z(+) 510.3 (MH⁺) | | | | | | | | | |
| 198 | 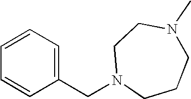 | —(CH₃)N (O)C— | CH | S | H | 0 | NH | H | Cl | F |
| | Synthetic Route: E Supporting Data: LCMS M/z(+) 496.24 (MH⁺) | | | | | | | | | |
| 199 | 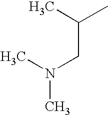 | —(O)C— | CH | S | H | 0 | NH | H | Cl | F |
| | Synthetic Route: E Supporting Data: LCMS M/z(+) 408 (MH⁺) | | | | | | | | | |
| 200 | 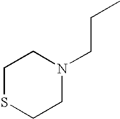 | HN(O)C— | CH | S | H | 0 | NH | H | Cl | F |
| | Synthetic Route: E Supporting Data: LCMS M/z(+) 452 (MH⁺) | | | | | | | | | |
| 201 | 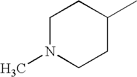 | HN(O)C— | CH | S | H | 0 | NH | H | Cl | F |
| | Synthetic Route: E Supporting Data: LCMS M/z(+) 467 (MH⁺) | | | | | | | | | |
| 202 | 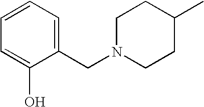 | HN(O)C— | CH | S | H | 0 | NH | H | 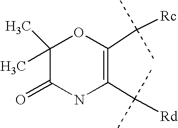 | |
| | Synthetic Route: B, C Supporting Data: LCMS M/z(+) 500 (MH⁺) | | | | | | | | | |
| 203 | 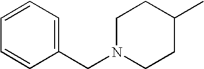 | HN(O)C— | CH | S | H | 0 | NH | H | —NHN═CH— | |
| | Synthetic Route: B, C Supporting Data: LCMS M/z(+) 484 (MH⁺) | | | | | | | | | |
| 204 | 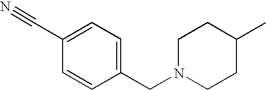 | HN(O)C— | CH | S | H | 0 | NH | H | —NHN═CH— | |
| | Synthetic Route: B, C Supporting Data: LCMS M/z(+) 509 (MH⁺) | | | | | | | | | |
| 205 | 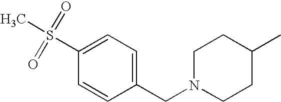 | HN(O)C— | CH | S | H | 0 | NH | H | —NHN═CH— | |
| | Synthetic Route: B, C Supporting Data: LCMS M/z(+) 560 (MH⁺) | | | | | | | | | |
| 206 | 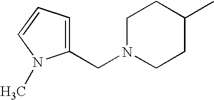 | HN(O)C— | CH | S | H | 0 | NH | H | —NHN═CH— | |
| | Synthetic Route: B, C Supporting Data: LCMS M/z(−) 485 (M − H) | | | | | | | | | |
| 207 | 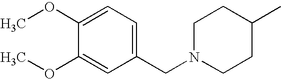 | HN(O)C— | CH | S | H | 0 | NH | H | —NHN═CH— | |
| | Synthetic Route: B, C Supporting Data: LCMS M/z(+) 544 (MH⁺) | | | | | | | | | |
| 208 | 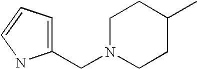 | HN(O)C— | CH | S | H | 0 | NH | H | —NHN═CH— | |
| | Synthetic Route: B, C Supporting Data: LCMS M/z(−) 471 (M − H) | | | | | | | | | |
| 209 | 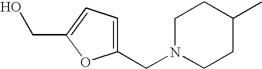 | HN(O)C— | CH | S | H | 0 | NH | H | —NHN═CH— | |
| | Synthetic Route: B, C Supporting Data: LCMS M/z(+) 504 (MH⁺) | | | | | | | | | |
| 210 | 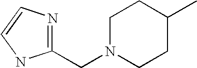 | HN(O)C— | CH | S | H | 0 | NH | H | —NHN═CH— | |
| | Synthetic Route: B, C Supporting Data: LCMS M/z(−) 472 (M − H) | | | | | | | | | |
| 211 | 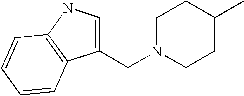 | HN(O)C— | CH | S | H | 0 | NH | H | —NHN═CH— | |
| | Synthetic Route: B, C Supporting Data: LCMS M/z(−) 521 (M − H) | | | | | | | | | |
| 212 | 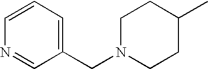 | HN(O)C— | CH | S | H | 0 | NH | H | —NHN═CH— | |
| | Synthetic Route: B, C Supporting Data: LCMS M/z(−) 483 (M − H) | | | | | | | | | |
| 213 | 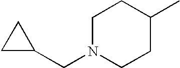 | HN(O)C— | CH | S | H | 0 | NH | H | —NHN═CH— | |
| | Synthetic Route: B, C Supporting Data: LCMS M/z(+) 448 (MH⁺) | | | | | | | | | |
| 214 | 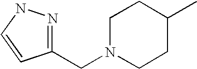 | HN(O)C— | CH | S | H | 0 | NH | H | —NHN═CH— | |
| | Synthetic Route: B, C Supporting Data: LCMS M/z(−) 472 (M − H) | | | | | | | | | |
| 215 | 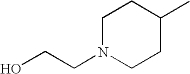 | HN(O)C— | CH | S | H | 0 | NH | H | —NHN═CH— | |
| | Synthetic Route: B, C Supporting Data: LCMS M/z(−) 436 (M − H) | | | | | | | | | |
| 216 | 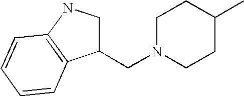 | HN(O)C— | CH | S | H | 0 | NH | H | —NHN═CH— | |
| | Synthetic Route: B, C Supporting Data: LCMS M/z(+) 523 (MH⁺) | | | | | | | | | |
| 217 | 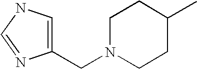 | HN(O)C— | CH | S | H | 0 | NH | H | —NHN═CH— | |
| | Synthetic Route: B, C Supporting Data: LCMS M/z(−) 472 (M − H) | | | | | | | | | |
| 218 | 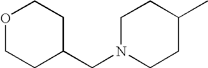 | HN(O)C— | CH | S | H | 0 | NH | H | —NHN═CH— | |
| | Synthetic Route: B, C Supporting Data: LCMS M/z(+) 492 (MH⁺) | | | | | | | | | |
| 219 | 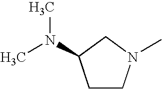 | HN(O)C— | CH | S | H | 0 | NH | H | —NHN═CH— | |
| | Synthetic Route: E Supporting Data: LCMS M/z(+) 420 (MH⁺) | | | | | | | | | |
| 220 | 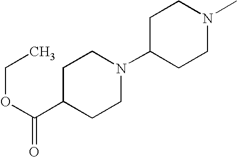 | —(O)C— | CH | S | H | 0 | NH | H | Cl | F |
| | Synthetic Route: E Supporting Data: LCMS M/z(+) 533.34 (MH⁺) | | | | | | | | | |
| 221 | 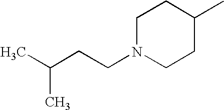 | —(O)C— | CH | S | H | 0 | NH | H | —NHCH═CH— | |
| | Synthetic Route: B, C Supporting Data: LCMS M/z(+) 476.24 (MH⁺) | | | | | | | | | |
| 222 | 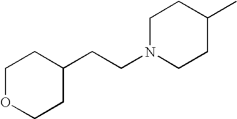 | HN(O)C— | CH | S | H | 0 | NH | H | Cl | F |
| | Synthetic Route: B, C Supporting Data: LCMS M/z(+) 518.23 (MH⁺) | | | | | | | | | |
| 223 | 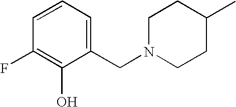 | HN(O)C— | CH | S | H | 0 | NH | H | Cl | F |
| | Synthetic Route: B, C Supporting Data: LCMS M/z(+) 518 (MH⁺) | | | | | | | | | |
| 224 | 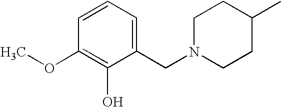 | HN(O)C— | CH | S | H | 0 | NH | H | —NHN═CH— | |
| | Synthetic Route: B, C Supporting Data: LCMS M/z(+) 530 (MH⁺) | | | | | | | | | |
| 225 | 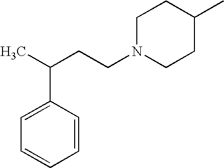 | HN(O)C— | CH | S | H | 0 | NH | H | —NHN═CH— | |
| | Synthetic Route: B, C Supporting Data: LCMS M/z(+) 526 (MH⁺) | | | | | | | | | |
| 226 | 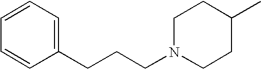 | HN(O)C— | CH | S | H | 0 | NH | H | —NHN═CH— | |
| | Synthetic Route: B, C Supporting Data: LCMS M/z(+) 512 (MH⁺) | | | | | | | | | |
| 227 | 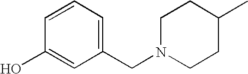 | HN(O)C— | CH | S | H | 0 | NH | H | —NHN═CH— | |
| | Synthetic Route: B, C Supporting Data: LCMS M/z(−) 498 (M − H) | | | | | | | | | |
| 228 | 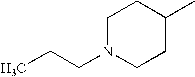 | HN(O)C— | CH | S | H | 0 | NH | H | —NHN═CH— | |
| | Synthetic Route: B, C Supporting Data: LCMS M/z(+) 436 (MH⁺) | | | | | | | | | |
| 229 | 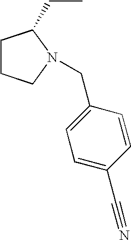 | HN(O)C— | CH | S | H | 0 | NH | H | —NHN═CH— | |
| | Synthetic Route: E, F, C Supporting Data: LCMS M/z(+) 509 (MH⁺) | | | | | | | | | |
| 230 | 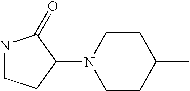 | HN(O)C— | CH | S | H | 0 | NH | H | Cl | F |

Synthetic Route: D Supporting Data: LCMS M/z(+) 489 (MH⁺)

TABLE 4

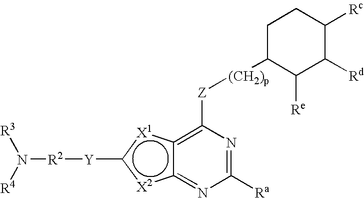

| No. | R⁴R³NR² | Y | X¹ | X² | R⁴ | p | Z | R^{e} | R^{d} | R^{c} |
|---|---|---|---|---|---|---|---|---|---|---|
| 231 | 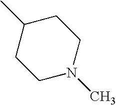 | HN(O)C— | CH | S | H | 0 | NH | H | CH₃ | H |

Synthetic Route: E
Supporting Data: LCMS M/z (+) 388 (MH⁺)

TABLE 5

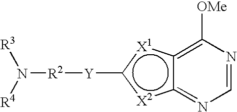

| No. | R⁴R³NR² | Y | X¹ | X² | R⁴ |
|---|---|---|---|---|---|
| 232 | 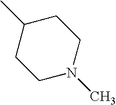 | HN(O)C— | CH | S | H |
| | Synthetic Route: E | | | | |
| | Supporting Data: NMR δ(CD₃SOCD₃) 1.48-1.64 (2H, | | | | |
| | m), 1.74-1.83 (2H, m), 2.01 (2H, t), 2.19 (3H, s), 2.75-2.84 | | | | |
| | (2H, m), 3.64-3.77 (1H, m), 4.10 (3H, s), 8.24 (1H, s), 8.68 | | | | |
| | (1H, d), 8.74 (1H, s); (LCMS M/z (+) 307 (MH⁺) | | | | |
| 233 | 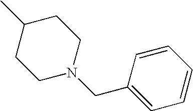 | HN(O)C— | CH | S | H |

Synthetic Route: E
Supporting Data: NMR δ(CD₃SOCD₃) 1.53-1.67 (2H,
m), 1.99-2.08 (2H, m), 2.12-2.23 (2H, m), 2.81-2.91 (2H, m),
3.52 (2H, s), 3.94-4.07 (1H, m), 4.15 (3H, s), 6.0 (1H, d),
7.23-7.35 (5H, m), 7.72 (1H, s), 8.69 (1H, s); LCMS M/z (+)
383 (MH⁺)

Compounds of formula (I) are suitably prepared by various routes which will be apparent to the chemist of ordinary skill. In particular compounds of formula (I) wherein Y is a group —NR⁸C(O)— may be obtained by reacting a compound of formula (IV) 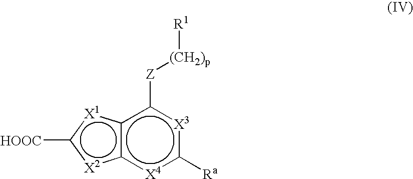
where R^{a}, R¹, X¹, X², X³, X⁴ and p are as defined in relation to formula (I), with a compound of formula (V) 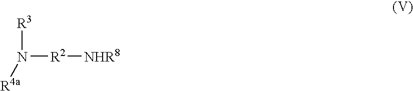
where R², R³ and R⁸ are as defined in relation to formula (I) and R^{4a} is a group R⁴ as defined in relation to formula (I), or a precursor thereof; and thereafter, if desired or necessary, converting any precursor groups R^{4a} to a group R⁴.

The reaction is suitably effected in an organic solvent such as dimethylformamide, in the presence of a base such as N,N-diisopropylethylamine and HATU at ambient temperature.

Examples of precursor groups R^{4a} include amine protecting groups such as tertiary butyloxycarbonyl (Boc) groups, which may be removed using conventional deprotection methods. Thereafter, the hydrogen group may be replaced by an alternative R⁴ group by an alkylation reaction or reductive amination reaction. Examples of such reactions are illustrated hereinafter.

Compounds of formula (IV) are suitably prepared by hydrolysis of a compound of formula (VI) 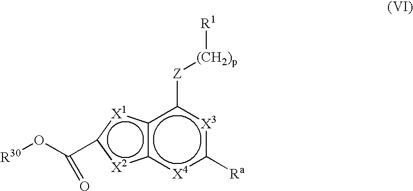
where R^{a}, R¹, X¹, X², X³, X⁴, Z and p are as defined in relation to formula (I), and R³⁰ is a hydrocarbyl group such as C₁₋₆alkyl.

Suitably hydrolysis is conducted in an organic solvent such as methanol, at temperatures such as 25 to 45° C. and using lithium hydroxide to effect hydrolysis.

Compounds of formula (VI) are suitably prepared by reacting a compound of formula (VII) 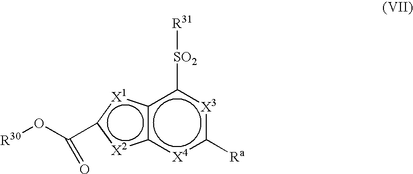
where X¹, X², X³, X⁴, Z and R^{a} are as defined in relation to formula (I), R³⁰ is as defined in relation to formula (VI) and R³¹ is a hydrocarbyl group such as C₁₋₆alkyl optionally substituted with a carboxylate ester group of formula COOR³⁵ where R³⁵ is a hydrocarbyl group such as a C₁₋₆alkyl group, with a compound of formula (VIII) 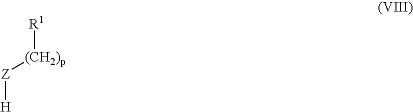
where R¹, Z and p are as defined in relation to formula (I).

The reaction is suitably effected in an adhydrous organic solvent such as propan-2-ol, in the presence of a base such as N,N-diisopropylethylamine. Temperatures in the range of from 60 to 100° C. are suitably employed.

Compounds of formula (VII) are suitably prepared by oxidation of a compound of formula (IX) 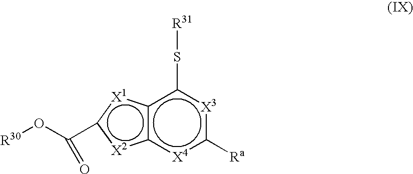
where X¹, X², X³, X⁴, Z and R^{a} are as defined in relation to formula (I), and R³⁰ is as defined in relation to formula (VI) and R³¹ is as defined in relation to formula (VII). Oxidation is suitably effected using an oxidising agents such as meta-chloroperoxybenzoic acid in an organic solvent such as dichloromethane at ambient temperature.

Compounds of formula (IX) where R³¹ is a group CH₂COOR³⁰ where R³⁰ is as defined in relation to formula (VI), X¹ is CH and X² is sulphur may be prepared by reacting a compound of formula (X) 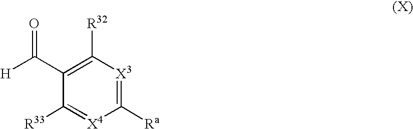
where X³ and X⁴ are as defined in relation to formula (I) and R³² and R³³ are leaving groups, such as halo groups, and in particular chloro, with a compound of formula (XI) 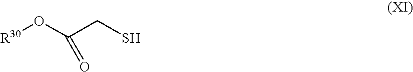
where R³⁰ is as defined in relation to formula (VII).

The reaction is suitably effected in an organic solvent such as dichloromethane, in the presence of a base such as N,N-diisopropylethylamine. Temperatures in the range of from −10° C. to ambient temperature are suitably employed.

Compounds of formula (X) may be prepared by reacting a compound of formula (XII) 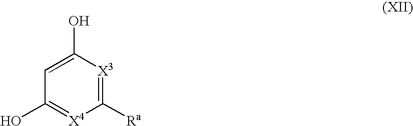
where R^{a}, X³ and X⁴ are as defined in relation to formula (I), with a halogenating agent, such as phosphorus oxychloride (POCl₃).

The reaction is suitably effected in an organic solvent such as N,N-dimethylformamide. Temperatures in the range of from 80 to 120° C. are suitably employed.

Alternatively, compounds of formula (IX) where one of X¹ or X² is CH and the other is S may be prepared by reacting a compound of formula (XIII) 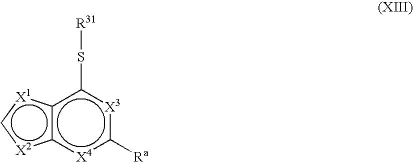
where X³ and X⁴ are as defined in relation to formula (I), R³¹ is as defined in relation to formula (VII), with a compound of formula (XIV) 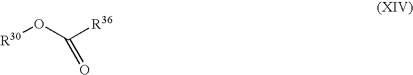
where R³⁰ is as defined in relation to formula (VI), R³¹ is as defined in relation to formula (VII) and R³⁶ is a leaving group such as halo, and in particular chloro.

The reaction is suitably effected in an anhydrous organic solvent such as tetrahydrofuran, in the presence of a base such as n-butyllithium. Temperatures in the range of from −65 to 0° C. are suitably employed.

Compounds of formula (XIII) may be prepared by reacting a compound of formula (XV) 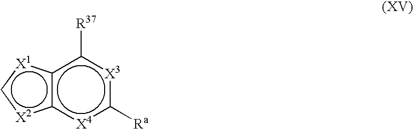
where one of X¹ and X² is CH and other is S, R^{a}, X³ and X⁴ are as defined in relation to formula (I) and R³⁷ is a leaving group such as halo, and in particular, chloro, with a compound of formula (XVI)
NaS—R³¹ (XVI)
The reaction is suitably effected in an organic solvent such as dichloromethane, using temperatures of from 25 to 60° C.

In an alternative route, compounds of formula (I) are prepared by reacting a compound of formula (XVII) 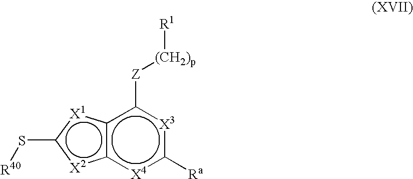
where R^{a}, R¹, X¹, X², X³, X⁴, Z and p are as defined in relation to formula (I) and R⁴⁰ is an alkyl group such as methyl, with a compound of formula (XVIII) 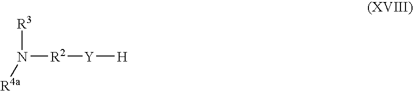
where R², R³ and Y are as defined in relation to formula (I) and R^{4a} is a group R⁴ as defined in relation to formula (I) or a precursor thereof, and thereafter if desired or necessary converting a precursor group R^{4a} to a group R⁴.

The reaction is suitably effected in an organic solvent such as isopropanol, in the presence of a base such as diisopropylethylamine. Temperatures in the range of from 60 to 100° C. are suitably employed. This route is particularly suitable where Y is a group such as —NR⁸—. When Y is a group such as —O— then a suitable base would be sodium hexamethyldisilylazide and a suitable solvent would be DMA.

Compounds of formula (XVII) may be prepared by reacting a compound of formula (XIX) 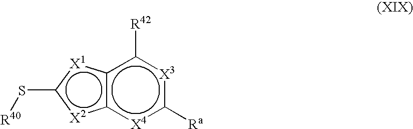
where R^{a}, X¹, X², X³ and X⁴ are as defined in relation to formula (I), R⁴⁰ is as defined in relation to formula (XVII) and R⁴² is a leaving group such as halo, and in particular chloro, with a compound of formula (VIII) as defined above. Suitable reaction conditions are similar to those described above for the reaction of compounds of formula (VII) with formula (VIII).

Compounds of formula (XIX) where R⁴² is halo such as chloro are suitably prepared by halogenating a compound of formula (XX) 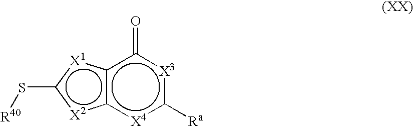
where X¹, X², X³, X⁴ and R^{a} are as defined in relation to formula (I) and R⁴⁰ is as defined in relation to formula (XVII), with a halogenating agent such as phosphorus oxychloride. Suitable reaction conditions are similar to those described above in relation to the halogenation of compounds of formula (XII).

Compounds of formula (XX) where R^{a} is hydrogen and X³ and X⁴ are both nitrogen are suitably prepared by reacting a compound of formula (XXI) 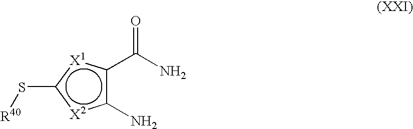
where X¹ and X² are as defined in relation to formula (I) and R⁴⁰ is as defined in relation to formula (XVII), with methanoic acid. The reaction is suitably effected in an organic solvent such as formic acid. Temperatures in the range of from 80 to 120° C. are suitably employed.

Where R^{a} is other than hydrogen, compounds of formula (XX) may be prepared by reacting a compound of formula (XXI) with a compound of formula (XXII)
R^{a}C(O)Cl (XXII)
where R^{a} is as defined in relation to formula (I). In this case, the reaction is suitably effected in an organic solvent such as tetrahydrofuran, in the presence of a base such as diisopropylethylamine. Temperatures in the range of from ambient to 80° C. are suitably employed.

Compounds of formula (XXI) where X¹ is S and X² is N are suitably prepared by reacting a compound of formula (XXIII) 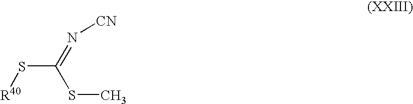
where R⁴⁰ is as defined in relation to formula (XVII), with a compound of formula (XXIV)
Cl—CH₂—C(O)—NH₂ (XXIV)
The reaction is suitably effected in an organic solvent such as ethanol. Temperatures in the range of from 40 to 100° C. are suitably employed. A base such as sodium methoxide is then added and heating continued.

In an alternative route, compounds of formula (I) are prepared by reacting a compound of formula (XXV) 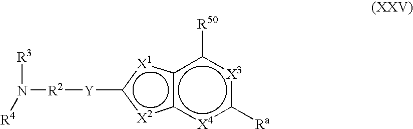
where R³, R⁴, R², Y, X¹, X², X³, X⁴ and R^{a} are as defined in relation to formula (I), provided that any amine groups are optionally protected, and R⁵⁰ is a leaving group, with a compound of formula (VIII) as defined above. The reaction is suitably carried out in an organic solvent, such as tetrahydrofuran, at low temperatures, for example of from 0-−100° C. An inert atmosphere, for instance an argon atmosphere, may be present. Examples of suitable leaving groups R⁵⁰ include halo such as chloro.

Thereafter, if desired, any protecting groups can be removed, using conventional methods.

In yet a further alternative method, compounds of formula (I) where R³ and R² together with the nitrogen to which they are attached form a heterocyclic ring, for instance so that the group of formula (x) above is a group of formula (bb)-(ff), they may be prepared by reacting a compound of formula (XXVI) 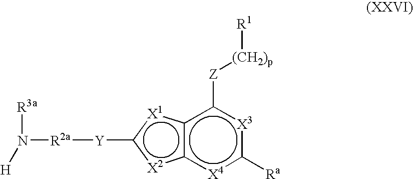
where R¹, Y, Z, X¹, X², X³, X⁴ p and R^{a} are as defined in relation to formula (I), R^{3a} and R^{2a} together with the nitrogen atom to which they are attached form a ring, with a compound of formula (XXVII)
R⁴—R⁵¹ (XXVII)
where R⁴ is as defined in relation to formula (I), and R⁵¹ is a leaving group, such as halo, and in particular bromo. The reaction is suitably carried out in an organic solvent such as dimethylformamide, in the presence of a base such as an alkali metal carbonate, for instance potassium carbonate. Moderate temperatures for example of from 0 to 50° C., and conveniently at ambient temperature, are suitably employed.

Alternatively, where R⁴ is an optionally substituted alkyl group, the compound of formula (XXVI) may be reacted with a compound of formula (XXVIII)
R^{4x}—C(O)H (XXVIII)
where a group R^{4x}—CH₂— is equivalent to the desired R⁴ group, in the presence of a mild reducing agent. This reaction is suitably effected in an organic solvent such as tetrahydrofuran at moderate temperatures for example of from 0 to 50° C., and conveniently at ambient temperature. A suitable dehydrating agent is magnesium sulphate.

In this case, the compounds of formula (XXVI) used is suitably in the form of a salt such as an acid addition salt, for example a trifluoroacetic acid salt.

Compounds of formula (XXVI) are suitably prepared by deprotecting a compound of formula (XXVIII) 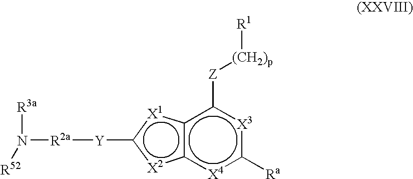
where X¹, X², X³, X⁴, R^{a}, Z, R¹ and p are as defined in relation to formula (I), R^{2a} and R^{3a} are as defined in relation to formula (XXVI) and R⁵² is an amine protecting group such as benzyloxycarbonyl (Boc) or tertiary butyloxycarbonyl. Suitable deprotection conditions would be apparent to a skilled person, but may include treatment with an acid such as acetic acid in the presence of hydrogen bromide, at moderate temperatures, or hydrochloric acid.

Compounds of formula (XXVIII) are suitably prepared by reacting a compound of formula (XXIX) 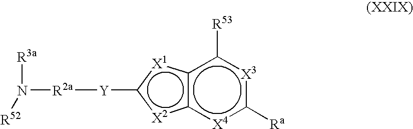
where X¹, X², X³, X⁴ and R^{a} are as defined in relation to formula (I), R^{2a} and R^{3a} are as defined in relation to formula (XXVI), R⁵² is as defined in relation to formula (XXVIII), and R⁵³ is a leaving group such as halo, and in particular chloro, with a compound of formula (VIII) as defined above. Suitable reaction conditions are analogous to those described above for the reaction of compounds of formula (VII) with a compound of formula (VIII).

Compounds of formula (XXIX) are suitably prepared by reacting a compound of formula (XXX) 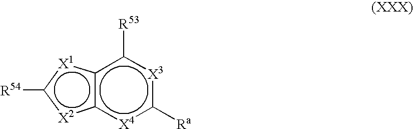
where X¹, X², X³, X⁴ and R^{a} are as defined in relation to formula (I), and R⁵³ is as defined in relation to formula (XXIX), and R⁵⁴ is a leaving group such as halo, and in particular chloro, with a compound of formula (XXXI) 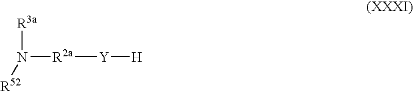
where R^{2a} and R^{3a} are as defined in relation to formula (XXX) and R⁵² is as defined in relation to formula (XXVIII). The reaction is suitably effected in a solvent such as tetrahydrofuran.

Compounds of formula (XXVIII) where Y is —NR⁸C(O)— may also be prepared by reacting a compound of formula (IV) as defined above, with a compound of formula (XXXII) 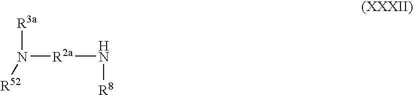
were R⁸ is as defined in relation to formula (I), R^{2a} and R^{3a} are as defined in relation to formula (XXX) and R⁵² is as defined in relation to formula (XXVIII), using conditions similar to those described above for the reaction of compounds of formula (IV) with formula (V).

Compounds of formula (I) where Z is a group —NR⁶C(O)— may also be prepared by reacting a compound of formula (XXXIII) 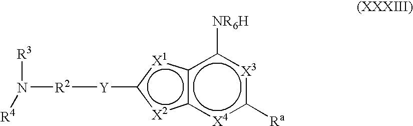
where R², R³, R⁴, R⁶, R^{a}, X¹, X², X³, X⁴ and Y are as defined in relation to formula (I), with a compound of formula (XXXIV) 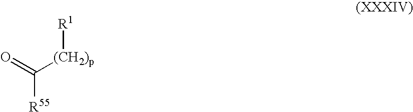
where p and R¹ are as defined in relation to formula (I) and R⁵⁵ is a leaving group such as halo, and in particular chloro.

The reaction is suitably effected in a solvent such as acetonitrile, dimethylsulphoxide (DMSO) or water, in the presence of a base such as diisopropylethylamine. Moderate temperatures, for example from 0 to 50° C. and conveniently, ambient temperatures are suitably employed.

Compounds of formula (XXXIII) may be prepared by deprotection of a compound of formula (XXXV) 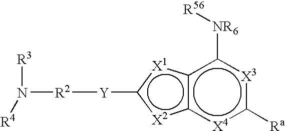
where R², R³, R⁴, R⁶, Y, X¹, X², X³, X⁴ and R^{a} are as defined in relation to formula (I) and R⁵⁶ is a nitrogen protecting group, such as a benzyl derivative, for instance 4-methoxybenzyl. Conditions suitable for the removal of the protecting group would be apparent to a chemist, but may include acidification for example using an organic acid such as trifluoroacetic acid at elevated temperatures, for instance of from 50-90° C., and in particular at about 70° C.

Compounds of formula (XXXV) may be prepared by methods analogous to those described above in relation to the preparation of compounds of formula (I). For example, where Y is —NHC(O)—, compounds of formula (XXXVI) 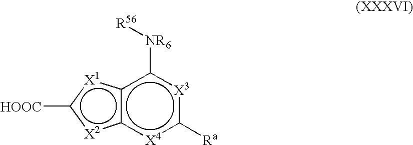
may be reacted with compounds of formula (V) as described above, using analogous conditions to those described for the reaction between compound (IV) and compound (V). Alternatively, the R⁴ group may be added in a subsequent reaction step, using reagents such as compounds of formula (XXVII) or (XXVIII), which are applied to the corresponding compound of formula (XXXV) where R⁴ is replaced with hydrogen.

The application of these methods to novel compounds of the invention forms a further aspect of the invention.

Compounds of formulae (V), (XXII), (XXIII), (XXVII), (XXVIII), (XXX), (XXXI) and (XXXIV) are either known compounds or they can be prepared from known compounds by conventional methods which would be readily apparent to a skilled chemist.

Variants of these processes may also be envisaged.

Any novel intermediates defined herein form a further aspect of the invention.

The invention further provides a compound of formula (I) as defined above for use in the treatment of inflammatory disease. When used in this way, the compounds are suitably formulated into pharmaceutical compositions which further contain a pharmaceutically acceptable carrier and these form a further aspect of the invention.

Furthermore, the invention provides the use of a compound of formula (I) as defined above in the preparation of a medicament for the treatment of inflammatory disease.

Some compounds of formula (I) may possess chiral centres. It is to be understood that the invention encompasses all such optical isomers and diasteroisomers of compounds of formula (I) and pharmaceutical compositions containing these.

The invention further relates to all tautomeric forms of the compounds of formula (I) and pharmaceutical compositions containing these.

It is also to be understood that certain compounds of the formula (I) can exist in solvated as well as unsolvated forms such as, for example, hydrated forms. It is to be understood that the invention encompasses all such solvated forms and pharmaceutical compositions containing these.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular or intramuscular dosing or as a suppository for rectal dosing).

The compositions of the invention may be obtained by conventional procedures using conventional pharmaceutical excipients, well known in the art. Thus, compositions intended for oral use may contain, for example, one or more colouring, sweetening, flavouring and/or preservative agents.

Suitable pharmaceutically acceptable excipients for a tablet formulation include, for example, inert diluents such as lactose, sodium carbonate, calcium phosphate or calcium carbonate, granulating and disintegrating agents such as corn starch or algenic acid; binding agents such as starch; lubricating agents such as magnesium stearate, stearic acid or talc; preservative agents such as ethyl or propyl p-hydroxybenzoate, and anti-oxidants, such as ascorbic acid. Tablet formulations may be uncoated or coated either to modify their disintegration and the subsequent absorption of the active ingredient within the gastrointestinal track, or to improve their stability and/or appearance, in either case, using conventional coating agents and procedures well known in the art.

Compositions for oral use may be in the form of hard gelatin capsules in which the active ingredient is mixed with an inert solid diluent, for example, calcium carbonate, calcium phosphate or kaolin, or as soft gelatin capsules in which the active ingredient is mixed with water or an oil such as peanut oil, liquid paraffin, or olive oil.

Aqueous suspensions generally contain the active ingredient in finely powdered form together with one or more suspending agents, such as sodium carboxymethylcellulose, methylcellulose, hydroxypropylmethylcellulose, sodium alginate, polyvinyl-pyrrolidone, gum tragacanth and gum acacia; dispersing or wetting agents such as lecithin or condensation products of an alkylene oxide with fatty acids (for example polyoxyethylene stearate), or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives (such as ethyl or propyl p-hydroxybenzoate, anti-oxidants (such as ascorbic acid), colouring agents, flavouring agents, and/or sweetening agents (such as sucrose, saccharine or aspartame).

Oily suspensions may be formulated by suspending the active ingredient in a vegetable oil (such as arachis oil, olive oil, sesame oil or coconut oil) or in a mineral oil (such as liquid paraffin). The oily suspensions may also contain a thickening agent such as beeswax, hard paraffin or cetyl alcohol. Sweetening agents such as those set out above, and flavouring agents may be added to provide a palatable oral preparation. These compositions may be preserved by the addition of an anti-oxidant such as ascorbic acid.

Dispersible powders and granules suitable for preparation of an aqueous suspension by the addition of water generally contain the active ingredient together with a dispersing or wetting agent, suspending agent and one or more preservatives. Suitable dispersing or wetting agents and suspending agents are exemplified by those already mentioned above. Additional excipients such as sweetening, flavouring and colouring agents, may also be present.

The pharmaceutical compositions of the invention may also be in the form of oil-in-water emulsions. The oily phase may be a vegetable oil, such as olive oil or arachis oil, or a mineral oil, such as for example liquid paraffin or a mixture of any of these. Suitable emulsifying agents may be, for example, naturally-occurring gums such as gum acacia or gum tragacanth, naturally-occurring phosphatides such as soya bean, lecithin, an esters or partial esters derived from fatty acids and hexitol anhydrides (for example sorbitan monooleate) and condensation products of the said partial esters with ethylene oxide such as polyoxyethylene sorbitan monooleate. The emulsions may also contain sweetening, flavouring and preservative agents.

Syrups and elixirs may be formulated with sweetening agents such as glycerol, propylene glycol, sorbitol, aspartame or sucrose, and may also contain a demulcent, preservative, flavouring and/or colouring agent.

The pharmaceutical compositions may also be in the form of a sterile injectable aqueous or oily suspension, which may be formulated according to known procedures using one or more of the appropriate dispersing or wetting agents and suspending agents, which have been mentioned above. A sterile injectable preparation may also be a sterile injectable solution or suspension in a non-toxic parenterally-acceptable diluent or solvent, for example a solution in 1,3-butanediol.

Suppository formulations may be prepared by mixing the active ingredient with a suitable non-irritating excipient which is solid at ordinary temperatures but liquid at the rectal temperature and will therefore melt in the rectum to release the drug. Suitable excipients include, for example, cocoa butter and polyethylene glycols.

Topical formulations, such as creams, ointments, gels and aqueous or oily solutions or suspensions, may generally be obtained by formulating an active ingredient with a conventional, topically acceptable, vehicle or diluent using conventional procedure well known in the art.

Compositions for administration by insufflation may be in the form of a finely divided powder containing particles of average diameter of, for example, 30 or much less, the powder itself comprising either active ingredient alone or diluted with one or more physiologically acceptable carriers such as lactose. The powder for insufflation is then conveniently retained in a capsule containing, for example, 1 to 50 mg of active ingredient for use with a turbo-inhaler device, such as is used for insufflation of the known agent sodium cromoglycate.

Compositions for administration by inhalation may be in the form of a conventional pressurised aerosol arranged to dispense the active ingredient either as an aerosol containing finely divided solid or liquid droplets. Conventional aerosol propellants such as volatile fluorinated hydrocarbons or hydrocarbons may be used and the aerosol device is conveniently arranged to dispense a metered quantity of active ingredient.

For further information on Formulation the reader is referred to Chapter 25.2 in Volume 5 of Comprehensive Medicinal Chemistry (Corwin Hansch; Chairman of Editorial Board), Pergamon Press 1990.

The amount of active ingredient that is combined with one or more excipients to produce a single dosage form will necessarily vary depending upon the host treated and the particular route of administration. For example, a formulation intended for oral administration to humans will generally contain, for example, from 0.5 mg to 2 g of active agent compounded with an appropriate and convenient amount of excipients which may vary from about 5 to about 98 percent by weight of the total composition. Dosage unit forms will generally contain about 1 mg to about 500 mg of an active ingredient. For further information on Routes of Administration and Dosage Regimes the reader is referred to Chapter 25.3 in Volume 5 of Comprehensive Medicinal Chemistry (Corwin Hansch; Chairman of Editorial Board), Pergamon Press 1990.

The size of the dose for therapeutic or prophylactic purposes of a compound of the Formula I will naturally vary according to the nature and severity of the conditions, the age and sex of the animal or patient and the route of administration, according to well known principles of medicine.

In using a compound of the Formula I for therapeutic or prophylactic purposes it will generally be administered so that a daily dose in the range, for example, 0.5 mg to 75 mg per kg body weight is received, given if required in divided doses. In general lower doses will be administered when a parenteral route is employed. Thus, for example, for intravenous administration, a dose in the range, for example, 0.5 mg to 30 mg per kg body weight will generally be used. Similarly, for administration by inhalation, a dose in the range, for example, 0.5 mg to 25 mg per kg body weight will be used. Oral administration is however preferred.

In a further aspect, the invention provides a method of treating inflammatory disease by administering a compound of formula (I) as described above, or a pharmaceutical composition as described above.

The invention is further illustrated, but not limited by the following Examples in which the following general procedures were used unless stated otherwise.

N,N-Dimethylformamide (DMF) was dried over 4 Å molecular sieves. Anhydrous tetrahydrofuran (THF) was obtained from Aldrich SURESEAL™ bottles. Other commercially available reagents and solvents were used without further purification unless otherwise stated. Organic solvent extracts were dried over anhydrous MgSO₄. ¹H, ¹³C and ¹⁹F NMR were recorded on Bruker WM200, WM250, WM300 or WM400 instruments using Me₂SO-d₆ with Me₄Si or CCl₃F as internal standard as appropriate, unless otherwise stated. Chemical shifts are in d (ppm) and peak multiplicities are designated as follows: s, singlet; d, doublet; dd, doublet of doublets; t, triplet; dt, doublet of triplets; q, quartet; m, multiplet; br, broad. Mass spectra were recorded on VG 12-12 quadrupole, VG 70-250 SE, VG ZAB 2-SE or a VG modified AEI/Kratos MS9 spectrometers. For TLC analysis, Merck precoated TLC plates (silica gel 60 F254, d=0.25 mm) were used. Flash chromatography was performed on silica (Merck Kieselgel: Art.9385). Melting point determinations were performed on a Kofler block or with a Büchi melting point apparatus and are uncorrected. All temperatures are in degrees Centigrade.

### EXAMPLE 1

Synthetic Route (A)

Preparation of Compound 7 in Table 2

### 4-Amino-2-methylsulfanyl-thiazole-5-carboxylic acid amide

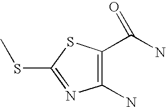 A mixture of cyanimidodithiocarbonic acid monomethyl ester monopotassium salt (30 g) and 2-chloroacetamide (16.6 g) were heated together in ethanol (60 ml) at reflux for 1.5 hours. The mixture was allowed to cool to room temperature before sodium methoxide (9.4 g) was added. The mixture was reheated and held at reflux for 3 hours. The mixture was cooled to room temperature and evaporated to dryness. Water (100 ml) was added and the solid filtered, washed with water and dried in vacuo at 50° C. to yield 4-amino-2-methylsulfanyl-thiazole-5-carboxylic acid amide (9.6 g).

### 2-Methylsulfanyl-4H-thiazolo[4,5-d]pyrimidin-7-one

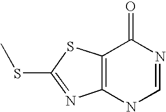 4-Amino-2-methylsulfanyl-thiazole-5-carboxylic acid amide (9.6 g) was dissolved in formic acid (30 ml) and the mixture heated at reflux for 14 hours. The mixture was then cooled to room temperature before water (100 ml) was added. The residue was filtered, washed with water and dried under vacuum at 60° C. to produce 2-methylsulfanyl-4H-thiazolo[4,5-d]pyrimidin-7-one (8.9 g). LCMS M/z(+) 199 (MH⁺).

### 7-Chloro-2-methylsulfanyl-thiazolo[4,5-d]pyrimidine

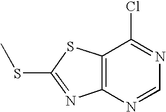

2-Methylsulfanyl-4H-thiazolo[4,5-d]pyrimidin-7-one was suspended as a slurry in phosphorus oxychloride (20 ml) and heated at 100° C. overnight. The majority of the remaining phosphorus oxychloride was evaporated in vacuo. The black residue was poured onto ice (200 g) and the resulting mixture stirred until the ice had melted. The residue was collected by filtration, washed with water and dried under vacuum at 40° C. to give 7-chloro-2-methylsulfanyl-thiazolo[4,5-d]pyrimidine as an ochre solid, (7.8 g). LCMS M/z(+) 217.9/219 (MH⁺)

### (3-Chloro-4-fluoro-phenyl)-(2-methylsulfanyl-thiazolo[4,5-d]pyrimidin-7-yl)-amine

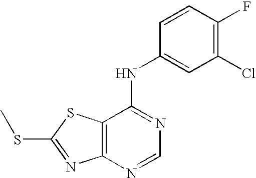 A mixture of 7-chloro-2-methylsulfanyl-thiazolo[4,5-d]pyrimidine (1.5 g), 4-fluoro-3-chloroaniline (1.1 g) and diisopropylethylamine (1.2 ml) were heated at 80° C. in isopropanol (10 ml) for 18 hours. The mixture was cooled and evaporated in vacuo to dryness.
The residue was collected by filtration and washed with isopropanol to produce (3-chloro-4-fluoro-phenyl)-(2-methylsulfanyl-thiazolo[4,5-d]pyrimidin-7-yl)-amine (1.1 g). LCMS M/z(+) 327 (MH⁺).

### N⁷-(3-Chloro-4-fluoro-phenyl-N²-(2-dimethylamino-ethyl)-thiazolo[4,5-d]pyrimidine-2,7-diamine

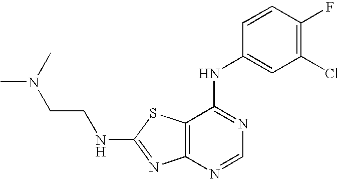 A mixture of (3-chloro-4-fluoro-phenyl)-(2-methylsulfanyl-thiazolo[4,5-d]pyrimidin-7-yl)-amine (0.16 g) and N,N-dimethylethylene diamine (0.9 g) was stirred at 100° C. in N-methylpyrrolidine (4 ml) for 18 hours. The resulting mixture was partitioned between ethyl acetate and water. The organic layer was separated, dried over sodium sulfate and evaporated to a gum. The gum was dissolved in dichloromethane, applied to a silica column and the product eluted using 20% methanol in dichloromethane, to produce N⁷-(3-Chloro-4-fluoro-phenyl)-N²-(2-dimethylamino-ethyl)-thiazolo[4,5-d]pyrimidine-2,7-diamine (0.055 g). LCMS M/z(+) 367 (MH⁺).

### EXAMPLE 2

Synthetic Route (B)

Preparation of Intermediate Compound

### Benzyl 4-{[(4-chlorothieno[2,3-d]pyrimidin-6-yl)carbonyl]amino}piperidine-1-carboxylate

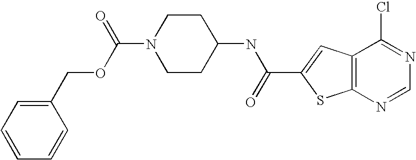 Lithium diisopropylethylamine (22 ml of a 2M solution in tetrahydrofuran, heptane and ethylbenzene) was added to a solution of 4-chloro-thieno[2,3-d]pyrimidine (7.2 g) in dry tetrahydrofuran (70 ml) at −60° C. under an atmosphere of argon. After stirring at −60° C. for 20 minutes, 4-isocyanato-piperidine-1-carboxylic acid benzyl ester (10 g) was added and the mixture was allowed to warm to ambient temperature before partitioning between water (200 ml) and ethylacetate (200 ml). The organic phase was dried (MgSO₄), concentrated in vacuo and purified by silica gel column chromatography using a gradient of 20% ethyl acetate/isohexane to 100% ethyl acetate as the eluant. The product was obtained as a cream solid (8.6 g).

¹H-NMR (CDCl₃): 1.39-1.55 (2H, m), 2.00-2.11 (2H, m), 2.99 (2H, t), 4.05-4.30 (3H, m), 5.12 (2H, s), 6.61 (1H, d), 7.27-7.37 (5H, m), 7.89 (1H, s), 8.89 (1H, s). LCMS M/z(+) 429 (M-H⁺)

### Benzyl 4-({[4-(1H-indazol-6-ylamino)thieno[2,3-d]pyrimidin-6-yl]carbonyl}amino)piperidine-1-carboxylate

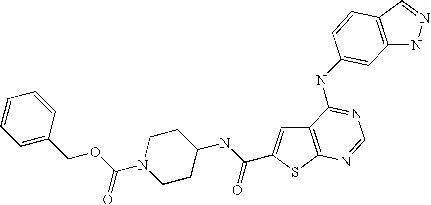 A mixture of benzyl 4-{[(4-chlorothieno[2,3-d]pyrimidin-6-yl)carbonyl]amino}piperidine-1-carboxylate (4.9 g), 6-aminoindazole (1.67 g) and N,N-diisopropylethylamine (3.96 ml) in anhydrous propan-2-ol (120 ml) was heated to 90° C. for 18 hours. After cooling to ambient temperature a yellow precipitate of benzyl 4-({[4-(1H-indazol-6-ylamino)thieno[2,3-d]pyrimidin-6-yl]carbonyl}amino)piperidine-1-carboxylate was isolated by filtration, washed with propan-2-ol and concentrated in vacuo to dryness (4.52 g).

¹H-NMR (DMSO-d6): 1.39-1.56 (2H, m), 1.81-1.91 (2H, m), 2.89-3.05 (2H, m), 3.93-4.08 (3H, m), 5.08 (2H, s), 7.28-7.47 (6H, m), 7.72 (1H, d), 7.99 (1H, s), 8.34 (1H, s), 8.43 (1H, s), 8.58-8.63 (2H, m), 9.95 (1H, s), 12.597 (1H, bs). LCMS M/z(+) 526 (MH⁻)

Other nucleophiles may be used in place of 6-aminoindazole as required to give compounds of formula (I).

### 4-(1H-Indazol-6-ylamino)-N-piperidin-4-ylthieno[2,3-d]pyrimidine-6-carboxamide

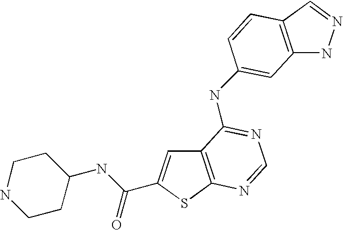 Benzyl 4-({[4-(1H-indazol-6-ylamino)thieno[2,3-d]pyrimidin-6-yl]carbonyl}amino)piperidine-1-carboxylate (4 g) was stirred at ambient temperature with 30% hydrogen bromide/acetic acid (30 ml) for 2 hours. The solvent was concentrated in vacuo to yield 4-(1H-indazol-6-ylamino)-N-piperidin-4-ylthieno[2,3-d]pyrimidine-6-carboxamide as a solid (4.4 g).

¹H-NMR (DMSO-d6): 1.70-1.88 (2H, m), 1.92-2.06 (2H, m), 2.96-3.11 (2H, m), 3.27-3.39 (2H, m) 3.99-4.15 (1H, m), 7.41-7.47 (1H, m), 7.72 (1H, d), 8.02 (1H, s), 8.31 (1H, s), 8.40-8.68 (4H, m), 8.79 (1H, d), 10.144 (1H, bs). LCMS M/z(+) 394 (M+).

### EXAMPLE 3

Synthetic Route (C)

Preparation of Compound No. 166 in Table 2

### 4-[(3-Chloro-4-fluorophenyl)amino]-N-[1-(1,2,3-thiadiazol-4-ylmethyl)piperidin-4-yl]thieno[2,3-d]pyrimidine-6-carboxamide

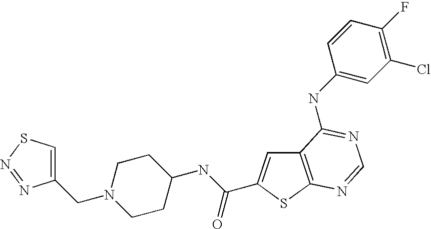 Sodium triacetoxyborohydride (150 mg) was added to a mixture of a trifluoroacetic acid salt of 4-[(3-chloro-4-fluorophenyl)amino]-N-piperidin-4-ylthieno[2,3-d]pyrimidine-6-carbothioamide (130 mgs), N,N-diisopropylethylamine (0.13 ml), 1,2,3-thiadiazole-4-carboxaldehyde (110 mg) and MgSO₄ (spatula full-weight not recorded) in anhydrous tetrahydrofuran (5 ml) at ambient temperature. The mixture was stirred at ambient temperature for 18 hours before partitioning between water (20 ml) and ethyl acetate (15 ml). The organic phase was dried (MgSO₄), concentrated in vacuo and purified by silica gel column chromatography using a gradient of dichloromethane to 5% methanol in dichloromethane as the eluant. 4-[(3-Chloro-4-fluorophenyl)amino]-N-[1-(1,2,3-thiadiazol-4-ylmethyl)piperidin-4-yl]thieno[2,3-d]pyrimidine-6-carboxamide was obtained as a white solid (135 mg).

¹H-NMR (DMSO-d6): 1.52-1.67 (2H, m), 1.75-1.86 (2H, m), 2.15 (2H, t), 2.84-2.94 (2H, m), 3.67-3.81 (1H, m), 4.02 (2H, s), 7.43 (1H, t), 7.72-7.80 (1H, m), 8.15-8.21 (1H, m), 8.32 (1H, s), 8.57-8.63 (2H, m), 9.02 (1H, s), 10.007 (1H, bs). LCMS M/z(+) 526 (MH⁺).

Compounds prepared by Route B (hydrogen bromide salts) or Route E (trifluoroacetate salts) were be used in place of 4-[(3-chloro-4-fluorophenyl)amino]-N-piperidin-4-ylthieno[2,3-d]pyrimidine-6-carbothioamide as required to give compounds of formula (I). Aldehydes and ketones were used in place of 1,2,3-thiadiazole-4-carboxaldehyde as required to further give compounds of formula (I).

### EXAMPLE 4

Synthetic Route (D)

Preparation of Compound No in 230 in Table 2

### 4-(3-Chloro-4-fluoro-phenylamino)-thieno[2,3-d]pyrimidine-6-carboxylic acid [1-(2-oxo-pyrrolidin-3-yl)-piperidin-4-yl]-amide

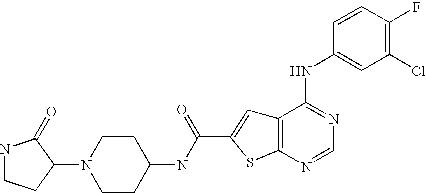

A solution of 4-(4-chloro-3-fluoro-phenylamino)-thieno[2,3-d]pyrimidine-6-carboxylic acid piperidin-4-ylamide (0.1 g) and 3-bromopyrolidinone (0.49 g) in dimethylformamide (1 ml) was stirred for 24 hours at ambient temperature in the presence of solid potassium carbonate (0.1 g). Water (5 ml) was added and the product extracted into ethyl acetate (5 ml).

After drying with sodium sulfate, the organic layer was evaporated in vacuo. The residue was purified using reverse phase HPLC eluting from 5-95% acetonitrile in water to produce 4-(3-Chloro-4-fluoro-phenylamino)-thieno[2,3-d]pyrimidine-6-carboxylic acid [1-(2-oxo-pyrrolidin-3-yl)-piperidin-4-yl]-amide (0.077 g). LCMS M/z(+) 489 (MH⁺)

Compounds prepared by Route B (hydrogen bromide salts) or Route E (trifluoroacetate salts) were be used in place of 4-(4-chloro-3-fluoro-phenylamino)-thieno[2,3-d]pyrimidine-6-carboxylic acid piperidin-4-ylamide as required to give compounds of formula (I). Bromides, chlorides and mesylates were used in place of 3-bromopyrrolidinone as required to further give compounds of formula (I).

### EXAMPLE 5

Example of Synthetic Route (E)

Preparation of Intermediate Useful in the Preparation of Compounds of the Invention

### 4-(Methylthio)thieno[2,3-d]pyrimidine

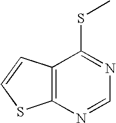 4-Chlorothieno[2,3-d]pyrimidine (84 g) and sodium methanethiolate (43.5 g) were stirred in dichloromethane (400 ml) at 40° C. for 88 hours. The mixture was then washed with water (2×500 ml), dried (MgSO₄) and concentrated in vacuo to give 4-(methylthio)thieno[2,3-d]pyrimidine as a pale brown solid (82.36 g). LCMS M/z(+) 183 (MH⁺)

### Methyl 4-(methylthio)thieno[2,3-d]pyrimidine-6-carboxylate

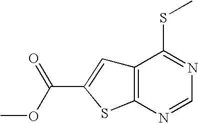 4-(Methylthio)thieno[2,3-d]pyrimidine (17.68 g) was dissolved in anhydrous tetrahydrofuran (300 ml) and stirred under argon at −65° C. n-Butyl lithium (1.6M in hexanes, 66 ml) then added dropwise over 30 minutes. Mixture left to stir at −65° C. for a further 30 minutes, then methyl chloroformate (9 ml) added dropwise over 5 minutes and mixture stirred and allowed to warm to ambient temperature over 16 hours. The resulting precipitate was filtered and washed with water (150 ml) and ethyl acetate (150 ml), then concentrated in vacuo to give methyl 4-(methylthio)thieno[2,3-d]pyrimidine-6-carboxylate as a yellow solid (14.23 g). LCMS M/z(+) 241 (MH⁺)

### Methyl 4-(methylsulfonyl)thieno[2,3-d]pyrimidine-6-carboxylate

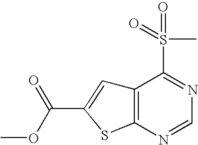 Methyl 4-(methylthio)thieno[2,3-d]pyrimidine-6-carboxylate (14.23 g) and m-chloroperoxybenzoic acid (22.51 g) were stirred in dichloromethane (400 ml) at ambient temperature for 16 hours. A further quantity of m-chloroperoxybenzoic acid (2.9 g) was then added and the mixture stirred for a further 16 hours at ambient temperature. The precipitate that formed was filtered off and the filtrate washed with sodium metabisulphate solution (300 ml) and saturated sodium bicarbonate solution (300 ml), then dried (MgSO₄), concentrated in vacuo and purified by flash chromatography, using dichloromethane as eluent to give methyl 4-(methylsulfonyl)thieno[2,3-d]pyrimidine-6-carboxylate as a pale yellow solid (9.1 g). LCMS M/z(+) 273 (MH⁺).

### 4-(3-Chloro-4-fluoro-phenylamino)-thieno[2,3-d]pyrimidine-6-carboxylic acid

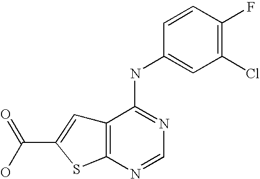 Methyl 4-(methylsulfonyl)thieno[2,3-d]pyrimidine-6-carboxylate (1.43 g), 4-fluoro-3-chloroaniline (4.5 g) and N,N-diisopropylethylamine (0.92 ml) were stirred in anhydrous propan-2-ol (50 ml) at 90° C. for 72 hours. The mixture was concentrated in vacuo and partitioned between methanol/dichloromethane (10:90, 150 ml) and 0.5 M aqueous sodium hydroxide solution (2×100 ml). The organic phase was dried (MgSO₄), concentrated in vacuo and the crude product was stirred in a mixture of lithium hydroxide monohydrate (800 mg), methanol (50 ml) and water (10 ml) at 60° C. for 90 minutes. The mixture was then concentrated in vacuo and partitioned between dichloromethane (100 ml) and water (50 ml) which gave an undissolved solid that was collected by filtration, re-suspended in water and neutralised to pH 7 with 2 M aqueous hydrochloric acid. The resulting precipitate was filtered, washed with water and dried under vacuum to give 4-(3-chloro-4-fluoro-phenylamino)-thieno[2,3-d]pyrimidine-6-carboxylic acid as a solid (350 mg).

LCMS M/z(+) 324 (MH⁺), RT=2.34

Other anilines, amines or oxygen nucleophiles such as sodium methoxide may be used in place of 4-fluoro-3-chloroaniline as required to give compounds of formula (I).

### 4-{[4-(4-Chloro-3-fluoro-phenylamino)-thieno[2,3-d]pyrimidine-6-carbonyl]-amino}-piperidine-1-carboxylic acid tert-butyl ester

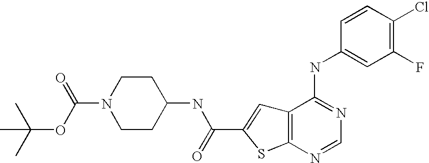 N,N-Diisopropylamine (2.5 ml) was added to a solution of 4-(3-chloro-4-fluoro-phenylamino)-thieno[2,3-d]pyrimidine-6-carboxylic acid (2 g), hydroxybenzotriazole (830 mg), 1-(3-dimethylaminopropyl)-3-ethylcarbodimide hydrochloride (1.5 g) and 1-Boc-4-amino piperidine hydrochloride (1.5 g) in dichloromethane (50 ml) at ambient temperature. The mixture was stirred at ambient temperature for 18 hours, after which time further quantities of 1-Boc-4-amino piperidine hydrochloride (500 mg), 1-(3-dimethylaminopropyl)-3-ethylcarbodiimide hydrochloride (500 mg) and N,N-diisopropylamine (0.8 ml) were added. The mixture was stirred for 2 days at 35° C., then partitioned between water and dichloromethane, the organic phase was dried (Na₂SO₄) and concentrated in vacuo to a gum. This was purified by silica gel chromatography to yield 4-{[4-(4-chloro-3-fluoro-phenylamino)-thieno[2,3-d]pyrimidine-6-carbonyl]-amino}-piperidine-1-carboxylic acid tert-butyl ester as a white solid (2.76 g).

¹H-NMR (DMSO-d6): 1.35-1.51 (11H, m), 1.75-1.85 (2H, m), 2.78-2.93 (2H, m), 3.88-4.00 (3H, m), 7.43 (1H, t), 7.72-7.80 (1H, m), 8.16-8.21 (1H, m), 8.33 (1H, s), 8.57-8.63 (2H, m), 10.00 (1H, s). LCMS M/z(+) 506/508 (MH⁺).

The product in this case is a BOC protected compound which could then be deprotected, for example using scheme F below. Reductive amination using schemes (C) or alkylation (D) above could be performed on it to produce other compounds of formula (I).

Other amines may be used in place of 1-Boc-4-amino piperidine hydrochloride as required to give compounds of formula (I) directly, without the need for these subsequent steps.

### EXAMPLE 6

Synthetic Route (F)

Preparation of Intermediate Useful in the Preparation of Compounds of the Invention

### 4-(4-Chloro-3-fluoro-phenylamino)-thieno[2,3-d]pyrimidine-6-carboxylic acid piperidin-4-ylamide

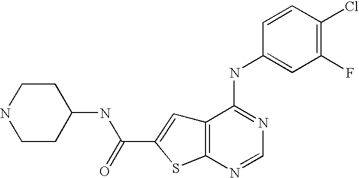 4-{[4-(4-Chloro-3-fluoro-phenylamino)-thieno[2,3-d]pyrimidine-6-carbonyl]-amino}-piperidine-1-carboxylic acid tert-butyl ester (2.75 g) was dissolved in a solution of 10 N hydrochloric acid in methanol (150 ml) and heated to 50° C. for 30 minutes. When evolution of gas has ceased, the mixture was concentrated in vacuo to produce 4-(4-chloro-3-fluoro-phenylamino)-thieno[2,3-d]pyrimidine-6-carboxylic acid piperidin-4-ylamide as a white solid (2.15 g).

¹H-NMR (DMSO-d6): 1.76-2.03 (4H, m), 2.91-3.08 (2H, m), 3.24-3.35 (2H, m), 4.00-4.11 (1H, m), 7.43 (1H, t), 7.78-7.85 (1H, m), 8.19-8.23 (1H, m), 8.60 (2H, d), 8.81-8.87 (1H, m), 9.02-9.23 (2H, m), 10.33 (1H, bs). LCMS M/z(−) 406/406 (M-H).

### EXAMPLE 7

Synthetic Route (G)

Preparation of Compound 145 in Table 2

### 4-(4-Methoxy-benzylamino)-thieno[2,3-d]pyrimidine-6-carboxylic acid (1-methyl-piperidin-4-yl)-amide

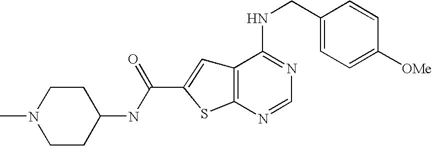 A mixture of 4-chloro-thieno[2,3-d]pyrimidine-6-carboxylic acid methyl ester (3.0 g), 4-methoxybenzylamine (1.8 g) and diisopropylethylamine (2.3 ml) was heated at 80° C. in isopropanol (10 ml) for 4 hours. After cooling, water (10 ml) followed by lithium hydroxide monohydrate (1.0 g) was added. Stirring was continued at room temperature for 72 hours. The solution was then acidified by addition of concentrated hydrochloric acid (10 N), and the resulting solid was collected by filtration, washed with water and dried under vacuum at 55° C. This solid was re-dissolved in dimethylformamide (20 ml) before HATU (4.2 g), diisopropylethylamine (1.9 ml) and N-methyl-4-aminopiperidine (1.27 g) were added. The mixture was stirred at ambient temperature for 4 hours, then the reaction mixture was partitioned between water and ethyl acetate. Concentration in vacuo produced 4-(4-methoxy-benzylamino)-thieno[2,3-d]pyrimidine-6-carboxylic acid (1-methyl-piperidin-4-yl)-amide as a solid (1.75 g). LCMS M/z(+) 412 (MH⁺).
4-Methanesulfonyl-thieno[2,3-d]pyrimidine-6-carboxylic acid methyl ester may be substituted for 4-chloro-thieno[2,3-d]pyrimidine-6-carboxylic acid methyl ester in this route.

### 4-Amino-thieno[2,3-d]pyrimidine-6-carboxylic acid (1-methyl-piperidin-4-yl)-amide

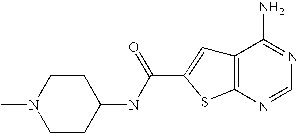 4-(4-Methoxy-benzylamino)-thieno[2,3-d]pyrimidine-6-carboxylic acid (1-methyl-piperidin-4-yl)-amide (1.3 g) was dissolved in trifluoroacetic acid (10 ml) and heated at 70° C. for 40 hours. The trifluoroacetic acid was then removed in vacuo and the residue washed with a mixture of ethyl acetate and aqueous sodium carbonate. The solid formed was filtered and washed with ethyl acetate and dried in vacuo to produce 4-amino-thieno[2,3-d]pyrimidine-6-carboxylic acid (1-methyl-piperidin-4-yl)-amide. (0.81 g). LCMS M/z(+) 292 (MH⁺).

### 4-(3-Chloro-4-fluoro-benzoylamino)-thieno[2,3-d]pyrimidine-6-carboxylic acid (1-methyl-piperidin-4-yl)-amide

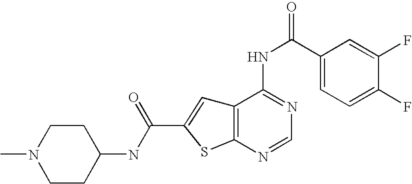 A mixture of 4-amino-thieno[2,3-d]pyrimidine-6-carboxylic acid (1-methyl-piperidin-4-yl)-amide (0.15 g; 0.5 mmol), diisopropylethylamine (0.12 ml) and 3,4-difluorobenzoyl chloride (0.176 g) was stirred in acetonitrile (2.0 ml) at ambient temperature for 18 hours, then concentrated in vacuo. The resulting residue was dissolved in a mixture of dimethylsulfoxide, acetonitrile and water and purified using reverse phase HPLC eluting from 5-95% acetonitrile in water, to produce 4-(3-4-difluoro-benzoylamino)-thieno[2,3-d]pyrimidine-6-carboxylic acid (1-methyl-piperidin-4-yl)-amide (0.01 g). LCMS M/z(+) 432 (MH⁺).

### EXAMPLE 8

Synthetic Route (H)

Preparation of Compound No. 165 in Table 2

### 5-Aminopyrimidine-4,6-dithiol

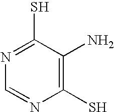 A suspension of dichloroaminopyrimidine (28.9 g) and sodium hydrosulfide monohydrate (52.1 g) in water (700 mL) was heated at reflux under an atmosphere of nitrogen. After 3 hours additional sodium hydro sulfide monohydrate (19.5 g) was added, heating continued for 3 hours then the mixture was allowed to cool to ambient temperature. Concentrated HCl was added to adjust the pH to 6-7, the resulting pale yellow precipitate filtered off then the filtrate was concentrated in vacuo to −500 mL. The filtrate was cooled (ice bath) and 2M HCl added to adjust the pH to 3, the resulting precipitate was filtered, washed with ice-cold water, dried under high-vacuum at 60° C. to give 5-aminopyrimidine-4,6-dithiol as a yellow solid (24 g). LCMS M/z(+) 160 (MH⁺).

### 7-Mercapto-thiazolo[5,4-d]pyrimidine-2-carboxylic acid ethyl ester

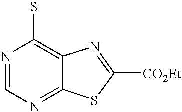 Ethyl oxalylchloride (280 μl) was added dropwise to an ice cooled solution of 5-aminopyrimidine-4,6-dithiol (300 mg) in pyridine (10 ml). The reaction was allowed to warm to room temperature and stirred for 4 hours. The mixture was concentrated in vacuo, azeotroped with toluene and the 7-mercapto-thiazolo[5,4-d]pyrimidine-2-carboxylic acid ethyl ester produced was used crude in the subsequent step.

### 7-Methylsulfanyl-thiazolo[5,4-d]pyrimidine-2-carboxylic acid

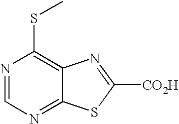 Crude 7-mercapto-thiazolo[5,4-d]pyrimidine-2-carboxylic acid ethyl ester was dissolved in 2 M NaOH (10 ml) and cooled (ice bath). Iodomethane (0.22 ml) was added dropwise and the mixture stirred at room temperature for 3 hours. The resultant solid 7-methylsulfanyl-thiazolo[5,4-d]pyrimidine-2-carboxylic acid (320 mg) was collected by filtration and dried in vacuum oven at 50° C.

¹H-NMR (DMSO): 2.7 (3H, s), 8.9 (1H, s). LCMS M/z(+) 228 (MH⁺).

### 7-Methylsulfanyl-thiazolo[5,4-d]pyrimidine-2-carboxylic acid methyl ester

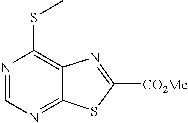 Acetyl chloride (0.5 ml) was added dropwise to an ice-cooled solution of methanol (10 ml). 7-Methylsulfanyl-thiazolo[5,4-d]pyrimidine-2-carboxylic acid was added and the reaction mixture stirred at room temperature overnight and then at reflux for a further 30 minutes. The reaction mixture was cooled to room temperature and concentrated under reduced pressure to give crude 7-methylsulfanyl-thiazolo[5,4-d]pyrimidine-2-carboxylic acid methyl ester (330 mg). LCMS M/z(+) 242 (MH⁺).

### 7-(3-Chloro-4-fluoro-phenylamino)-thiazolo[5,4-d]pyrimidine-2-carboxylic acid methyl ester

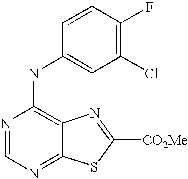 m-Chloroperoxybenzoic acid (472 mg) was added in one portion to a suspension of 7-methylsulfanyl-thiazolo[5,4-d]pyrimidine-2-carboxylic acid methyl ester (330 mg) in dichloromethane (20 ml) under an inert atmosphere. The mixture was stirred at room temperature for 4.5 hours. 1,4-Dioxane (20 ml) was then added followed by 3-chloro-4-fluoroaniline (300 mg) and the reaction stirred at room temperature overnight. The resulting mixture was concentrated in vacuo and subjected to chromatography (bond elute 10 g, eluting with 5% methanol/dichloromethane to yield an oily residue of 7-(3-chloro-4-fluoro-phenylamino)-thiazolo[5,4-t]pyrimidine-2-carboxylic acid methyl ester (260 mg); LCMS M/z(+) 338 (MH⁺).

### 7-(3-Chloro-4-fluoro-phenylamino)-thiazolo[5,4-d]pyrimidine-2-carboxylic acid

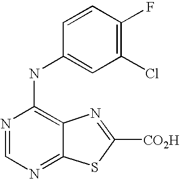 7-(3-Chloro-4-fluoro-phenylamino)-thiazolo[5,4-d]pyrimidine-2-carboxylic acid methyl ester (160 mg) was suspended in tetrahydrofuran (5 ml) and 2M sodium hydroxide (3 ml) and stirred at room temperature for 1 hour. The mixture was concentrated in vacuo and the mixture acidified with citric acid. The resultant precipitate of 7-(3-chloro-4-fluoro-phenylamino)-thiazolo[5,4-d]pyrimidine-2-carboxylic acid was collected by filtration and dried (210 mg). LCMS M/z(+) 324 (MH⁺).

### 7-(3-Chloro-4-fluoro-phenylamino)-thiazolo[5,4-d]pyrimidine-2-carboxylic acid (1-methyl-piperidin-4-yl)-amide

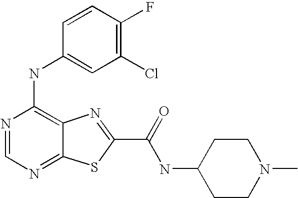 HATU (495 mg), N,N-diisopropylamine (450 ml) and 1-methylpiperidine-4-amine (150 mg) were added to a solution of 7-(3-chloro-4-fluoro-phenylamino)-thiazolo[5,4-d]pyrimidine-2-carboxylic acid (210 mg) in N,N-dimethylformamide (10 ml) and stirred at room temperature overnight. Water (10 ml) was added and the reaction mixture extracted with dichloromethane (2×15 ml). The combined organics were washed with brine (10 ml), dried (MgSO₄), filtered and concentrated in vacuo. The residue was subjected to chromatography (10 g bond elute, eluting with 20% methanol/dichloromethane+1% ammonia) to yield 7-(3-chloro-4-fluoro-phenylamino)-thiazolo[5,4-d]pyrimidine-2-carboxylic acid (1-methyl-piperidin-4-yl)-amide as a brown solid (43 mg).

¹H-NMR (DMSO, 373K): 1.9 (2H, m), 2.1 (2H, m), 2.8 (3H, s), 3.2 (2H, m), 3.4 (2H, m), 4.1 (1H, m), 7.4 (1H, t), 7.8 (1H, m), 8.1 (1H, m), 8.3 (1H, d), 8.6 (1H, s), 9.8 (1H, s). LCMS M/z(+) 421 (MH⁺).

### EXAMPLE 9

### trans-4-(4-Fluorophenylamino)-thieno[2,3-d]pyrimidine-6-carboxylic acid (1-benzyl-3-hydroxy-piperidin-4-yl)-amide

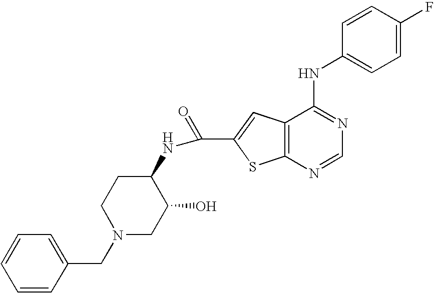 trans-4-(4-Fluorophenylamino)-thieno[2,3-d]pyrimidine-6-carboxylic acid (3-hydroxy-piperidin-4-yl)-amide hydrochloride (97 mg) was suspended in THF (5 ml). To this was added diisopropylethylamine (0.08 ml), benzaldehyde (0.047 ml) and sodium triacetoxyborohydride (97 mg). The reaction mixture was stirred at room temperature overnight. LCMS indicated incomplete reaction so more sodium triacetoxyborohydride (97 mg) was added and the reaction stirred for a further 2 h. The reaction was quenched by addition of methanol (5 ml) and evaporated. The residue was purified by silica gel column chromatography using a gradient of 0% EtOAc in isohexane to EtOAc as the eluant to give trans-4-(4-fluoro-phenylamino)-thieno[2,3-d]pyrimidine-6-carboxylic acid (1-benzyl-3-hydroxy-piperidin-4-yl)-amide as an off-white solid (50 mg).

¹H NMR (400.132 MHz, DMSO) 1.55 (m, 1H), 1.83 (m, 1H), 2.01 (m, 1H), 2.79 (d, 1H), 2.95 (m, 1H), 3.46 (d, 1H), 3.52-3.68 (m, 4H), 4.75 (d, 1H), 7.21-7.37 (m, 7H), 7.85 (dd, 2H), 8.37 (s, 1H), 8.46 (d, 1H), 8.53 (s, 1H), 9.95 (s, 1H). LCMS M/z(+) 478 (MH⁺).

### trans-4-(4-Fluorophenylamino)-thieno[2,3-d]pyrimidine-6-carboxylic acid (3-hydroxy-piperidin-4-yl)-amide hydrochloride

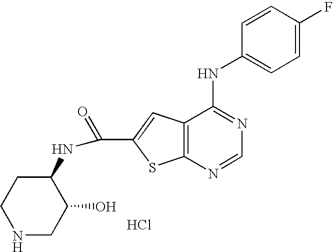 A preformed solution of acetyl chloride (2.5 ml) in methanol (25 ml) was added to trans-4-{[4-(4-fluorophenylamino)-thieno[2,3-d]pyrimidine-6-carbonyl]-amino}-3-hydroxypiperidine-1-carboxylic acid tert-butyl ester (114 mg) and the resultant solution stirred at room temperature overnight. The solution was evaporated to give trans-4-(4-fluorophenylamino)-thieno[2,3-d]pyrimidine-6-carboxylic acid (3-hydroxy-piperidin-4-yl)-amide hydrochloride as a yellow solid (97 mg).

¹H NMR (400.132 MHz, DMSO) 1.79 (m, 1H), 2.08 (m, 1H), 2.83 (m, 1H), 3.03 (m, 1H), 3.27-3.35 (m, 2H), 3.87 (m, 1H), 3.98 (m, 1H), 7.24 (t, 2H), 7.88 (m, 2H), 8.53 (s, 1H), 8.55 (s, 1H), 8.72 (d, 1H), 8.95 (s, 1H), 9.21 (s, 1H), 10.02 (s, 1H). LCMS M/z(+) 388 (MH⁺).

### trans-4-{[4-(4-Fluorophenylamino)-thieno[2,3-d]pyrimidine-6-carbonyl]-amino}-3-hydroxy-piperidine-1-carboxylic acid tert-butyl ester

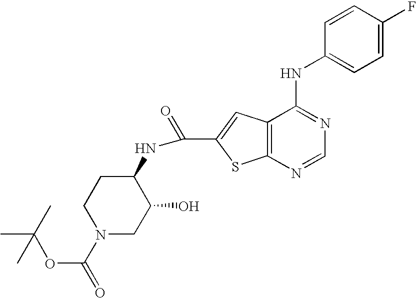 To a solution of 7-oxa-3-aza-bicyclo[4.1.0]heptane-3-carboxylic acid benzyl ester (4.29 g) in ethanol (100 ml) was added di-tert-butyl dicarbonate (4.0 g). The flask was purged with argon before addition of 10% palladium on carbon (1.0 g). The atmosphere was replaced with hydrogen and the reaction stirred at room temperature overnight. The solution was filtered, washing with a little methanol, and evaporated. The residue was purified by silica gel column chromatography using a gradient of isohexane to 40% EtOAc in isohexane as the eluant. 7-Oxa-3-aza-bicyclo[4.1.0]heptane-3-carboxylic acid tert-butyl ester was obtained as an oil (2.34 g).
7-Oxa-3-aza-bicyclo[4.1.0]heptane-3-carboxylic acid tert-butyl ester (545 mg) was dissolved in dioxane (5 ml). To this solution was added diisopropylethyamine (0.57 ml), benzylamine (0.324 ml) and ytterbium (III) trifluoromethanesulphonate (10 mg). The solution was sealed and heated to 140° C. for 20 min in a microwave oven. The solution was evaporated and the residue was purified by silica gel column chromatography using a gradient of 50% EtOAc in isohexane to EtOAc as the eluant. trans-4-Benzylamino-3-hydroxypiperidine-1-carboxylic acid tert-butyl ester and trans-3-benzylamino-4-hydroxypiperidine-1-carboxylic acid tert-butyl ester were obtained as an oil. LCMS M/z(+) 307 (MH⁺). This mixture was dissolved in ethanol (20 ml) and the reaction flask purged with argon. 10% Palladium on carbon (100 mg) was added and the atmosphere replaced with hydrogen. The reaction mixture was stirred at room temperature overnight. The suspension was filtered and evaporated to give a mixture of trans-4-amino-3-hydroxy-piperidine-1-carboxylic acid tert-butyl ester and trans-3-amino-4-hydroxy-piperidine-1-carboxylic acid tert-butyl ester as a white solid, which was used without purification. The mixture of hydroxyamines (302 mg) was added to a solution of 4-(4-fluorophenylamino)-thieno[2,3-d]pyrimidine-6-carboxylic acid (404 mg), diisopropylethylamine (0.49 ml) and HATU (530 mg) in DMF (7 ml). The reaction mixture was stirred at room temperature overnight. The reaction mixture was partitioned between EtOAc (50 ml) and water (100 ml). The organic phase washed with brine (50 ml), dried (Na₂SO₄) and evaporated. The residue was purified by silica gel column chromatography using a gradient of 20% EtOAc in isohexane to EtOAc as the eluant to give trans-4-{[4-(4-fluorophenylamino)-thieno[2,3-d]pyrimidine-6-carbonyl]-amino}-3-hydroxypiperidine-1-carboxylic acid tert-butyl ester (114 mg) as an oil.

¹H NMR (400.132 MHz, DMSO) 1.43 (s, 9H), 1.85 (d, 1H), 2.57-2.65 (, 1H), 2.76-2.87 (m, 1H), 3.43-3.49 (m, 1H), 3.79-3.90 (m, 2H), 3.99-4.07 (m, 1H), 5.06 (d, 1H), 7.23 (t, 2H), 7.86 (dd, 2H), 8.38 (s, 1H), 8.49 (d, 2H), 8.54 (s, 1H), 9.95 (s, 1H) (1 proton obscured.). LCMS M/z(+) 488 (MH⁺).

### 4-(4-Fluorophenylamino)-thieno[2,3-d]pyrimidine-6-carboxylic acid

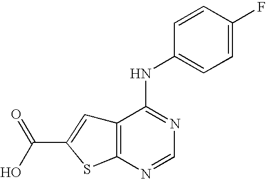 To a suspension of 4-chlorothieno[2,3-d]pyrimidine-6-carboxylic acid (10.0 g) in 2-propanol (60 ml) was added N,N-diisopropylethylamine (8 ml) and 4-fluoroaniline (4.4 ml). The resultant solution was heated at 85° C. overnight. The reaction mixture was allowed to cool and evaporated. The residue was diluted with water (100 ml) and acidified with concentrated hydrochloric acid. The resultant precipitate was filtered, washed with water and dried to give the product as a green solid, 13 g.

¹H NMR (DMSO) 7.22-7.26 (m, 2H), 7.84-7.89 (m, 2H), 8.56 (s, 1H), 8.64 (s, 1H), 9.99 (s, 1H). LCMS M/z(+) 290 (MH⁺).

### EXAMPLE 10

### N-(1-Benzylpiperidin-4-yl)-2-amino-4-[(4-fluorophenyl)amino]thieno[2,3-d]pyrimidine-6-carboxamide

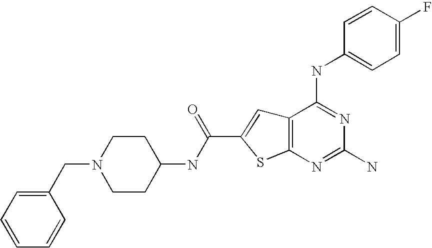 Methyl 2-amino-4-[(4-fluorophenyl)amino]thieno[2,3-d]pyrimidine-6-carboxylate (160 mg) and lithium hydroxide monohydrate (24 mg) were dissolved in methanol (5 ml) and THF (5 ml) and stirred under reflux for 4 hours. The mixture was allowed to cool, concentrated in vacuo and the residue dried under vacuum. It was then taken up in N,N-dimethylformamide (10 ml) and stirred at room temperature under argon. N-methylmorpholine (0.28 ml) was added followed by isobutyl chloroformate (0.072 ml). After 15 minutes, 4-amino-1-benzylpiperidine (0.11 ml) was added. Stirring was continued overnight with the argon source removed and the mixture was concentrated in vacuo. The residue was adsorbed onto silica and subjected to chromatography (12 g Redisep® cartridge, eluting with 0-15% methanol/dichloromethane) to give example N-(1-benzylpiperidin-4-yl)-2-amino-4-[(4-fluorophenyl)amino]thieno[2,3-d]pyrimidine-6-carboxamide as a white solid (37 mg).

¹H-NMR (DMSO, 373K): 1.5 (m, 2H), 1.8 (m, 2H), 2.0 (m, 2H), 2.8 (d, 2H), 3.5 (s, 2H), 3.7 (m, 1H), 6.6 (s, 2H), 7.1 (m, 2H), 7.3 (m, 5H), 7.9 (m, 2H), 8.1 (m, 2H), 9.4 (s, 1H). LCMS M/z(+) 477 (MH⁺).

### Methyl 2-amino-4-[(4-fluorophenyl)amino]thieno[2,3-d]pyrimidine-6-carboxylate

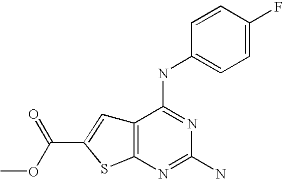 Methyl 2-amino-4-chlorothieno[2,3-d]pyrimidine-6-carboxylate (240 mg), 4-fluoroaniline (0.104 ml), concentrated hydrochloric acid (10 drops) and methanol (5 ml) were sealed in a microwave vessel and microwaved at 140° C. for 45 minutes. The mixture was allowed to cool to room temperature and the resulting precipitate filtered off and washed with cold methanol (5 ml) to give methyl 2-amino-4-[(4-fluorophenyl)amino]thieno[2,3-d]pyrimidine-6-carboxylate as a pale yellow powder (162 mg).

¹H-NMR (DMSO, 373K): 3.9 (s, 3H), 7.2 (t, 2H), 7.9 (m, 2H), 8.6 (s, 1H), 10.1 (m, 1H). LCMS M/z(+) 319 (MH⁺).

### Methyl 2-amino-4-chlorothieno[2,3-d]pyrimidine-6-carboxylate

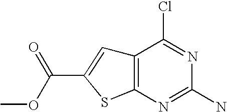 2-Amino-4,6-dichloro-5-formylpyrimidine (2.8 g), methyl thioglycolate (1.304 ml) and potassium carbonate (6.05 g) were suspended in acetonitrile (120 ml) and stirred at reflux under argon overnight. The mixture was allowed to cool to room temperature and concentrated in vacuo. The residue was triturated once with water (100 ml) and filtered. The solid material obtained was collected and azeotroped once with toluene and triturated once with dichloromethane (50 ml) to give methyl 2-amino-4-chlorothieno[2,3-d]pyrimidine-6-carboxylate as a yellow solid (2.78 g).

¹H-NMR (DMSO, 373K): 3.85 (s, 3H), 7.6 (s, 2H), 7.75 (s, 1H). LCMS M/z(+) 244 (MH⁺).

### EXAMPLE 11

Biological Assays for:

a) MCP-1 Mediated Calcium Flux in THP-1 Cells

The human monocytic cell line THP-1 was grown in a synthetic cell culture medium RPMI 1640 supplemented with 10% foetal calf serum, 6 mM glutamine and Penicillin-Streptomycin (at 50IU/ml penicillin, 50 μg streptomycin/ml, Gibco BRL). THP-1 cells were washed in assay buffer comprising of HBSS with Ca²⁺ and Mg²⁺ (without phenol red) (Gibco BRL)+20 mM HEPES+0.71 mg/ml Propenecid+2 mls/litre CaCl₂ 1M (BDH)+0.3 mg/ml BSA (Sigma) pH 7.4 and resuspended in the same buffer at a density of 1×10⁶ cells/ml. The cells were then loaded with assay buffer+1 mM FLUO-4 (molecular probes) for 40 min at 37° C., washed twice in assay buffer, and resuspended at 2×10⁵ cells/ml. 100 μl of the cell suspension was added to the wells of black clear-bottomed 96 well plates, to give 2×10⁴ cells/well. Cells were pelleted by centrifugation and washed with assay buffer. 100 ul of buffer+50 ul of compound was added to wells and incubated for 20 mins at (37° C.). Fluorescence was recorded using a FLIPR (FLuorometric Imaging Plate Reader—Molecular Devices). Cells were stimulated by addition of hMCP-1 to the wells.

Stimulation of THP-1 cells with hMCP-1 induced a rapid, transient rise in [Ca²⁺]i in a specific and dose dependent manner. Dose response curves indicated an approximate EC₅₀ of 4 nm. Compounds were dissolved in DMSO (10 mM) and were assayed for inhibition of calcium release over concentration ranges starting at 10 μM.

Certain compounds described above were tested in this screen and found to be active. For example, compound No. 74 in Table 2 had an IC50 of 0.379 μM and compound No. 128 in Table 2 had an IC50 of 0.313 μM.

b) hMCP-1 Receptor-Binding Assay

i) Cloning and Expression of hMCP-1 Receptor

The MCP-1 receptor B (CCR2B) cDNA was cloned by PCR from THP-1 cell RNA using suitable oligonucleotide primers based on the published MCP-1 receptor sequences (Charo et al., 1994, Proc. Natl. Acad. Sci. USA, 91, 2752). The resulting PCR products were cloned into vector PCR-II™ (InVitrogen, San Diego, Calif.). Error free CCR2B cDNA was subcloned as a Hind III-Not I fragment into the eukaryotic expression vector pcDNA3 (InVitrogen) to generate pcDNA3/CC-CKR2A and pcDNA3/CCR2B respectively.

Linearised pcDNA3/CCR2B DNA was transfected into CHO-K1 cells by calcium phosphate precipitation (Wigler et al., 1979, Cell, 16, 777). Transfected cells were selected by the addition of Geneticin Sulphate (G418, Gibco BRL) at 1 mg/ml, 24 hours after the cells had been transfected. Preparation of RNA and Northern blotting were carried out as described previously (Needham et al., 1995, Prot. Express. Purific., 6, 134). CHO-K1 clone 7 (CHO—CCR2B) was identified as the highest MCP-1 receptor B expresser.

ii) Preparation of Membrane Fragments

CHO—CCR2B cells were grown in DMEM supplemented with 10% foetal calf serum, 2 mM glutamine, 1× Non-Essential Amino Acids, 1× Hypoxanthine and Thymidine Supplement and Penicillin-Streptomycin (at 50 μg streptomycin/ml, Gibco BRL). Membrane fragments were prepared using cell lysis/differential centrifugation methods as described previously (Siciliano et al., 1990, J. Biol. Chem., 265, 19658). Protein concentration was estimated by BCA protein assay (Pierce, Rockford, Ill.) according to the manufacturer's instructions.

iii) Assay

¹²⁵I-labeled MCP-1 was prepared using Bolton and Hunter conjugation (Bolton et al., 1973, Biochem. J., 133, 529; Amersham International plc].

Test compounds were dissolved in DMSO and further diluted in assay buffer (50 mM HEPES, 1 mM CaCl₂, 5 nM MgCl₂, 0.03% BSA, pH 7.2) to give a range of concentrations starting with a top final concentration of 10 uM. All incubations had a 100 ul final volume and a DMSO concentration of 1%. Incubations contained 200 pM ¹²⁵I-labeled MCP-1 (Amersham Pharmacia), 2.5 mg/ml Scintillation proximity assay beads (Amersham Pharmacia RPNQ) and approx 5 ug CHO—CCR2B cell membranes. Non-specific binding was determined by the inclusion of a 1 uM unlabelled MCP-1 in the place of test compound. Total binding was determined in the presence of 1% DMSO without compound. Incubations were performed in sealed optiplates and kept at room temperature for 16 hours after which the plates were counted on a Packard TopCount (Packard TopCount™). Dose-response curves were generated from duplicate date points and IC₅₀ values were calculated using GraphPad Prizm® software. Percent inhibitions were calculated for single concentrations of compound by using the following formula 100−((compound binding minus non-specific binding)/(total binding minus non-specific binding)×100).

In the above assay each compound set out in the Examples below showed an IC₅₀ value of better than 20 μmol.

### EXAMPLE 12

Pharmaceutical Compositions

This Example illustrates, but is not intended to limit, representative pharmaceutical dosage forms of the invention as defined herein (the active ingredient being termed “Compound X”), for therapeutic or prophylactic use in humans:

### EXAMPLE A

(a)

| Tablet I | mg/tablet |
|---|---|
| Compound X. | 100 |
| Lactose Ph.Eur | 182.75 |
| Croscarmellose sodium | 12.0 |
| Maize starch paste (5% w/v paste) | 2.25 |
| Magnesium stearate | 3.0 |

(b)

| Tablet II | mg/tablet |
|---|---|
| Compound X | 50 |
| Lactose Ph.Eur | 223.75 |
| Croscarmellose sodium | 6.0 |
| Maize starch | 15.0 |
| Polyvinylpyrrolidone (5% w/v paste) | 2.25 |
| Magnesium stearate | 3.0 |

(c)

| Tablet III | mg/tablet |
|---|---|
| Compound X | 1.0 |
| Lactose Ph.Eur | 93.25 |
| Croscarmellose sodium | 4.0 |
| Maize starch paste (5% w/v paste) | 0.75 |
| Magnesium stearate | 1.0 |

(d)

| Capsule | mg/capsule |
|---|---|
| Compound X | 10 |
| Lactose Ph.Eur | 488.5 |
| Magnesium | 1.5 |

(e)

| Injection I | (50 mg/ml) |
|---|---|
| Compound X | 5.0% w/v |
| 1M Sodium hydroxide solution | 15.0% v/v |
| 0.1M Hydrochloric acid | to adjust pH to 7.6 |
| Polyethylene glycol 400 | 4.5% w/v |
| Water for injection | to 100% |

| Injection II | (10 mg/ml) |
|---|---|
| Compound X | 1.0% w/v |
| Sodium phosphate BP | 3.6% w/v |
| 0.1M Sodium hydroxide solution | 15.0% v/v |
| Water for injection | to 100% |

(g)

| Injection III | (1 mg/ml, buffered to pH6) |
|---|---|
| Compound X | 0.1% w/v |
| Sodium phosphate BP | 2.26% w/v |
| Citric acid | 0.38% w/v |
| Polyethylene glycol 400 | 3.5% w/v |
| Water for injection | to 100% |

(h)

| Aerosol I | mg/ml |
|---|---|
| Compound X | 10.0 |
| Sorbitan trioleate | 13.5 |
| Trichlorofluoromethane | 910.0 |
| Dichlorodifluoromethane | 490.0 |

(i)

| Aerosol II | mg/ml |
|---|---|
| Compound X | 0.2 |
| Sorbitan trioleate | 0.27 |
| Trichlorofluoromethane | 70.0 |
| Dichlorodifluoromethane | 280.0 |
| Dichlorotetrafluoroethane | 1094.0 |

(j)

| Aerosol III | mg/ml |
|---|---|
| Compound X | 2.5 |
| Sorbitan trioleate | 3.38 |
| Trichlorofluoromethane | 67.5 |
| Dichlorodifluoromethane | 1086.0 |
| Dichlorotetrafluoroethane | 191.6 |

(k)

| Aerosol IV | mg/ml |
|---|---|
| Compound X | 2.5 |
| Soya lecithin | 2.7 |
| Trichlorofluoromethane | 67.5 |
| Dichlorodifluoromethane | 1086.0 |
| Dichlorotetrafluoroethane | 191.6 |

(l)

| Ointment | ml | |
|---|---|---|
| Compound X | 40 | mg |
| Ethanol | 300 | μl |
| Water | 300 | μl |
| 1-Dodecylazacycloheptan-2-one | 50 | μl |
| Propylene glycol | to 1 | ml |

Note:

Compound X in the above formulations may comprise a compound as illustrated in herein.

The above formulations may be obtained by conventional procedures well known in the pharmaceutical art. The tablets (a)-(c) may be enteric coated by conventional means, for example to provide a coating of cellulose acetate phthalate. The aerosol formulations (h)-(k) may be used in conjunction with standard, metered dose aerosol dispensers, and the suspending agents sorbitan trioleate and soya lecithin may be replaced by an alternative suspending agent such as sorbitan monooleate, sorbitan sesquioleate, polysorbate 80, polyglycerol oleate or oleic acid.

1. The use of a compound of formula (I) 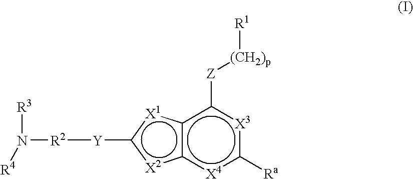
or a pharmaceutically acceptable salt or solvate thereof,
wherein X¹ or X² are selected from sulphur, nitrogen or CH, provided that at least one of X¹ or X² is sulphur or nitrogen;
one of X³ or X⁴ is nitrogen and the other is N or CH;
R^{a} is hydrogen, C₁₋₃alkyl, C₂₋₄alkenyl, C₂₋₄alkynyl, trifluoromethyl, halo, amino, C₁₋₃alkylamino, di-C₁₋₃alkylamino, C₁₋₄alkoxy, hydroxy, thioC₁₋₄alkyl, or cyclopropyl;
p is 0 or an integer selected from 1, 2, 3 or 4;
R¹ is hydrogen, or an optionally substituted cycloalkyl or optionally substituted aryl ring, wherein two substituents may be joined together to form an optionally substituted fused bicyclic ring, which may contain heteroatoms,
Z is oxygen or a group NR⁶ or —NR⁶C(O)— where R⁶ is hydrogen or C₁₋₆alkyl, or R⁶ is a C₂₋₆alkylene or C₂₋₆alkenylene group that is bonded to the ring R¹ to form a fused bicyclic ring system;
Y is a direct bond or a group, —O—, —C(O)—, —S(O)ₘ, —NR⁸—, —NR⁸C(O)—, —C(O)NR⁸—, S(O)ₘNR⁸— or —NR⁸S(O)ₘ—, where m is 0, 1 or 2 and R⁸ is hydrogen or an optionally substituted C₁₋₄alkyl group,
R² is a direct bond, an optionally substituted C₁₋₁₀straight or branched alkylene group, which is optionally interposed with a group NR^{b} where R^{b} is hydrogen or a C₁₋₃methyl group; or R² together with R⁸ may form an optionally substituted cycloalkyl or heterocyclic ring,
R³ and R⁴ are independently selected from an optionally substituted C₁₋₁₀ alkyl group, an optionally substituted C₂₋₁₀ alkenyl group, an optionally substituted C₁₋₁₀ alkynyl group or an optionally substituted heterocyclic group,
or R³ and R⁴ together with the nitrogen atom to which they are attached form an optionally substituted heterocyclic ring, which optionally contains additional heteroatoms,
or R³ together with R² or R⁸ and the nitrogen atom(s) to which they are attached form an optionally substituted heterocyclic ring which optionally contains additional heteroatoms,
or R³ and R⁴ together with R² form an optionally substituted bridged ring structure, in the preparation of a medicament for the treatment of C—C chemokine mediated conditions.

2. The use according to claim 1 wherein in the compound of formula (I), one of X¹ or X² is sulphur and the other is nitrogen or CH.

3. The use according to claim 1 or claim 2 wherein, in the compound of formula (I), R^{a} is hydrogen, methyl, trifluoromethyl or amino, and preferably R^{a} is hydrogen.

4. The use according to any one of the preceding claims wherein p is 0 or 1.

5. The use according to any one of the preceding claims wherein in the compounds of formula (I), R¹ is optionally substituted phenyl.

6. The use according to any one of claims 1 to 4 wherein, in the compound of formula (I), R¹ is a fused bicyclic ring of formula (i) 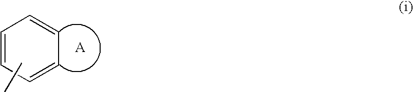
where A is an optionally substituted 4-7 membered ring which may contain one or more heteroatoms.

7. The use according to any one of the preceding claims wherein, in the compound of formula (I), Z is a group NR⁶ where R⁶ is as defined in claim 1.

8. The use according to any one of the preceding claims wherein, in the compound of formula (I), Y is selected from —O—, —C(O)—, —NH—, —NHCO—, —N(CH₃)C(O)—, or —CONH—.

9. The use according to claim 8 wherein Y is —NHCO—.

10. The use according to any one of the preceding claims, wherein in the compound of formula (I), R⁴R³N— comprise a group of sub-formula (xx)-(xxv). 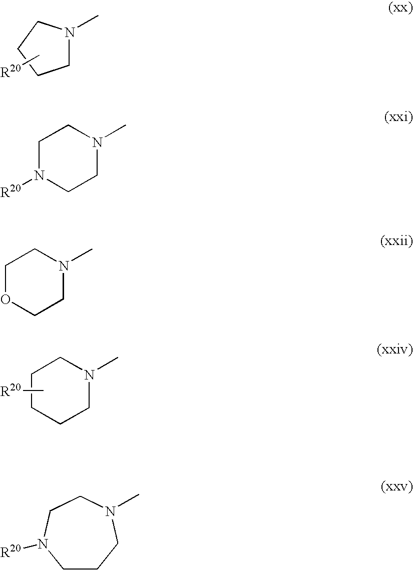
where R²⁰ is hydrogen or a substituent selected from alkyl, aralkyl such as benzyl, optionally substituted heterocyclic groups, and functional groups.

11. The use according to any one of claims 1 to 9 wherein in the compound of formula (I), the group of sub-formula (x) 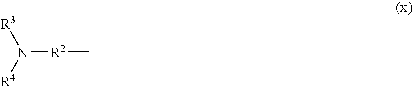
is a group of sub-formula (bb), (cc), (dd), (ee) or (ff) 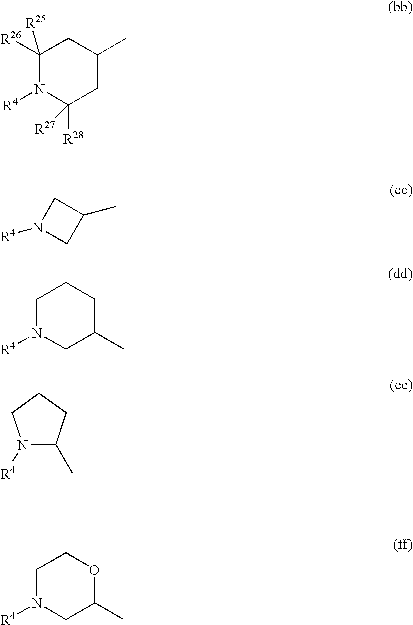
where R⁴ is as defined in claim 1, and R²⁵, R²⁶, R²⁷ and R²⁸ are independently selected from hydrogen or C₁₋₃alkyl.

12. The use according to claim 11 wherein in the compound of formula (I), the group of sub-formula (x) is a group of formula (bb) above.

13. A compound of formula (IG) 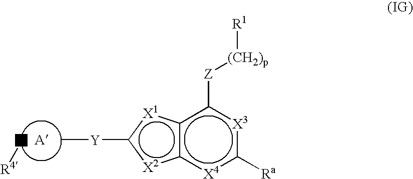
or a pharmaceutically acceptable salt or solvate thereof,
wherein X¹, X², X³, X⁴, R^{a}, p, R¹, Z and Y are as defined in relation to formula (I), the ring A′ is an optionally substituted heterocyclic ring which optionally contains further heteroatoms, and R⁴ is a substituted C₁₋₁₀ alkyl group, provided that at least one substituent on the group R^{4′} is selected from optionally substituted heterocyclyl, substituted aryl, a cycloalkyl group, a group C(O)R¹¹ or a group S(O)_{q}R¹¹ where R¹¹ is selected from hydrogen, optionally substituted hydrocarbyl or optionally substituted heterocyclyl, and q is 0 or an integer selected from 1, 2 or 3.

14. A compound of formula (IH) 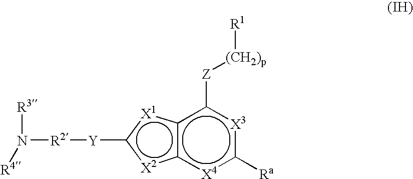
or a pharmaceutically acceptable salt or solvate thereof,
wherein X¹, X², X³, X⁴, R^{a}, p, R¹, Z and Y are as defined in relation to formula (I) R^{2′} is a C₁₋₁₀straight or branched alkylene group, which is optionally interposed with a group NR^{b} where R^{b} is hydrogen or a C₁₋₃methyl group; or R^{2′} together with any R⁸ group present in Y may form an optionally substituted cycloalkyl or heterocyclic ring, and R^{3″} and R^{4″} together with the nitrogen atom to which they are attached form a substituted heterocyclic ring, which optionally contains additional heteroatoms.

15. A compound of formula (IJ) 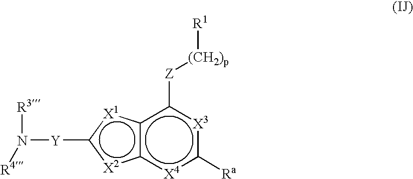
or a pharmaceutically acceptable salt or solvate thereof,
wherein X¹, X², X³, X⁴, R^{a}, p, R¹, Z, and Y are as defined in relation to formula (I) R^{3′″} and R^{4′″} together with the nitrogen atom to which they are attached form a heterocyclic ring, which optionally contains additional heteroatoms, and which is substituted by at least one group selected from (a) alkyl substituted by an optionally substituted heterocyclyl, (b) alkyl substituted by a substituted aryl group, (c) alkyl substituted by a cycloalkyl group, (d) a group C(O)R¹¹ or (e) a group S(O)_{q}R¹¹ where q and R¹¹ are as defined above.

16. A compound of formula (IK) 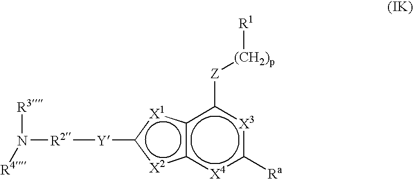
or a pharmaceutically acceptable salt or solvate thereof,
wherein X¹, X², X³, X⁴, R^{a}, p, R¹ and Z are as defined in claim 1,
Y′ is a group —NR^{8′}—, —NR^{8′}C(O)—, —C(O)NR^{8′}—, S(O)ₘNR^{8′}— or —NR^{8′}S(O)ₘ—, where m is 0, 1 or 2
and R^{2″} together with R^{8′} forms an optionally substituted cycloalkyl or heterocyclic ring,
R^{3″″} and R^{4″″} are independently selected from a substituted C₁₋₁₀ alkyl group (provided that at least one substitutent is other than hydroxy), an optionally substituted C₂₋₁₀ alkenyl group, an optionally substituted C₁₋₁₀ alkynyl group or an optionally substituted heterocyclic group,
or R^{3″″} and R^{4″″} together with the nitrogen atom to which they are attached form an optionally substituted heterocyclic ring, which optionally contains additional heteroatoms.

17. A compound of formula (IL) 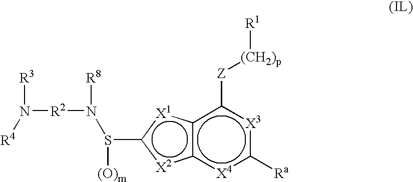
or a pharmaceutically acceptable salt or solvate thereof,
wherein X¹, X², X³, X⁴, p, R^{a}, Z, R², R³, R⁴, R⁸ and m are as defined in claim 1.

18. A compound of formula (IM) 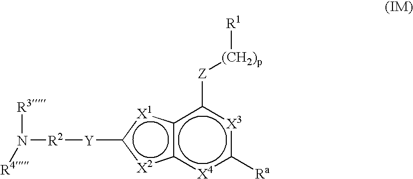
or a pharmaceutically acceptable salt or solvate thereof,
wherein X¹, X² X³, X⁴, R^{a}, p, R¹, Z, Y and R² are as defined in claim 1 and R^{3′″″} and R^{4′″″} are independently selected from an optionally substituted C₁₋₁₀ alkyl group, au optionally substituted C₂₋₁₀ alkenyl group, an optionally substituted C₁₋₁₀ alkynyl group or an optionally substituted heterocyclic group, provided that at least one of R^{3′″″} or R^{4′″″} is other than optionally substituted alkyl.

19. A compound of formula (IN) 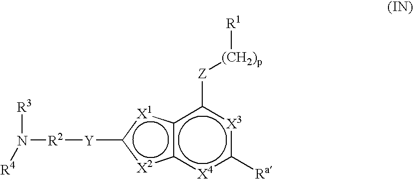
or a pharmaceutically acceptable salt or solvate thereof,
wherein X¹, X² X³, X⁴, R³, R⁴ p, R¹, Z, Y and R² are as defined in relation to formula (I), and R^{a} is C₂₋₄alkenyl, C₂₋₄alkynyl, trifluoromethyl, or cyclopropyl.

20. A compound of formula (IP) 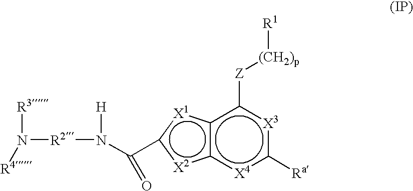
where R¹, p, Z, R^{a}, X¹, X², X³ and X⁴ are as defined in claim 1, R^{2′″} is an alkylene group, which together with R^{3′″″″} and the nitrogen atom to which they are attached form a heterocyclic ring, and R^{4′″″″} is a heterocyclic group which is substituted by at least one substituted alkyl group, and which optionally contains further substituents.

21. A compound according to claim 20 of formula (IPa) 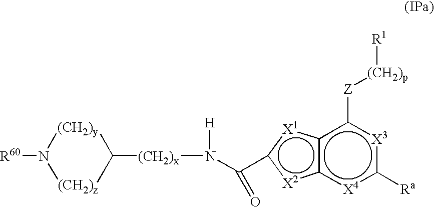
where R¹, p, Z, R^{a}, X¹, X², X³ and X⁴ are as defined in claim 1, and R⁶⁰ is a substituted C₁₋₁₀ alkyl group, an optionally substituted C₂₋₁₀ alkenyl group, an optionally substituted C₁₋₁₀ alkynyl group or an optionally substituted heterocyclic group;
x is 0, 1 or 2; y and z are independently selected from 0, 1, 2, 3, 4 or 5, provided that y+z is in the range of 2 to 7.

22. A process for preparing a compound according to any one of claims 13 to 21, which is selected from:
(a) where Y or the equivalent group is a group —C(O)NR⁸—, reacting a compound of formula (IV) 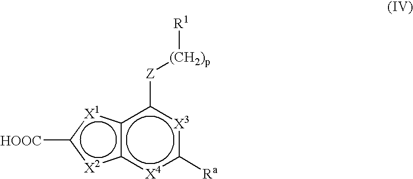
where R^{a}, R¹, X¹, X², X³, X⁴, Z and p are as defined in claim 1, with a compound of formula (V) 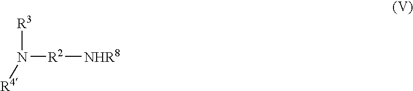
where R², R³ and R⁸ are as defined in claim 1 and R^{4′} is a group R⁴ as defined in claim 1, or a precursor thereof; and thereafter, if desired or necessary, converting any precursor groups R^{4′} to a group R⁴;
(b) by reacting a compound of formula (XVII) 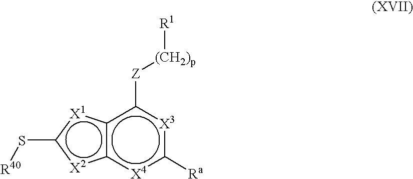
where R^{a}, R¹, X¹, X², X³, X⁴, Z and p are as defined in claim 1 and R⁴⁰ is an alkyl group, with a compound of formula (XVIII) 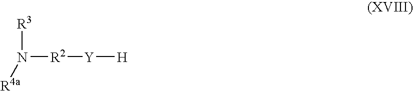
where R², R³ and Y are as defined in claim 1 and R^{4a} is a group R⁴ as defined in relation to formula (I) or a precursor thereof, and thereafter if desired or necessary converting a precursor group R^{4a} to a group R⁴;
(c) reacting a compound of formula (XXV) 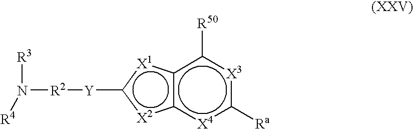
where R³, R⁴, R², Y, X¹, X², X³, X⁴ and R^{a} are as defined in claim 1, provided that any amine groups are optionally protected, and R⁵⁰ is a leaving group, with a compound of formula (VIII) 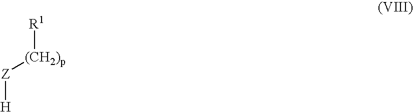
where R¹, Z and p are as defined in relation to formula (I);
(d) for the preparation of compounds of formula (I) where R³ and R² together with the nitrogen to which they are attached form a heterocyclic ring, reacting a compound of formula (XXVI) 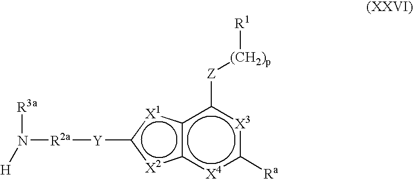
where R¹, Y, Z, X¹, X², X³, X⁴ p and R^{a} are as defined in claim 1, R^{3a} and R^{2a} together with the nitrogen atom to which they are attached form a ring, with a compound of formula (XXVII)
R⁴—R⁵¹ (XXVII)
where R⁴ is as defined in claim 1, and R⁵¹ is a leaving group,
or where R⁴ is an optionally substituted alkyl group, the compound of formula (XXVI) or a salt thereof may be reacted with a compound of formula (XXVIII)
R^{4x}—C(O)H (XXVIII)
where a group R^{4x}—CH₂— is equivalent to the desired R⁴ group, in the presence of a mild reducing agent; or
(e) for the preparation of compounds of formula (I) where Z is a group —NR⁶C(O)—, reacting a compound of formula (XXXIII) 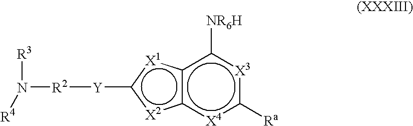
where R², R³, R⁴, R⁶, R^{a}, X¹, X², X³, X⁴ and Y are as defined in claim 1,
with a compound of formula (XXXIV) 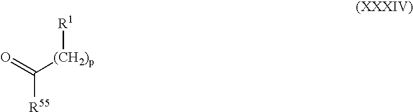
where p and R¹ are as defined in claim 1 and R⁵⁵ is a leaving group.

23. A compound, salt or solvate according to any one of claims 13 to 21 for use in therapy.

24. A pharmaceutical composition comprising a compound according to any one of claims 13 to 21 in combination with a pharmaceutically acceptable carrier or diluent.

25. A method for treating a C—C chemokine mediated disease, which method comprises administering to a patient in need thereof, a compound of formula (I) as defined in claim 1.

26. The use according to any one of claims 1 to 12 wherein the compound of formula (I) is selected from:
N⁷-(3-chloro-4-fluorophenyl)-N²-(1-propylpiperidin-4-yl)[1,3]thiazolo[4,5-d]pyrimidine-2,7-diamine,
N⁷-(3-chloro-4-fluorophenyl)-N²-[1-(2-methoxyethyl)piperidin-4-yl][1,3]thiazolo[4,5-d]pyrimidine-2,7-diamine,
N⁷-(3-chloro-4-fluorophenyl)-N²-(2-pyrrolidin-1-ylethyl)[1,3]thiazolo[4,5-d]pyrimidine-2,7-diamine,
N⁷-(3-chloro-4-fluorophenyl)-N²-(1-methylpiperidin-4-yl)[1,3]thiazolo[4,5-d]pyrimidine-2,7-diamine,
N⁷-(3-chloro-4-fluorophenyl)-N²-(2-piperazin-1-ylethyl)[1,3]thiazolo[4,5-d]pyrimidine-2,7-diamine,
N⁷-(3-chloro-4-fluorophenyl)-N²-(3-morpholin-4-ylpropyl) [1,3]thiazolo[4,5-d]pyrimidine-2,7-diamine,
N⁷-(3-chloro-4-fluorophenyl)-N²-[2-(dimethylamino)ethyl][1,3]thiazolo[4,5-d]pyrimidine-2,7-diamine,
N⁷-(3-chloro-4-fluorophenyl)-N²-[2-(diethylamino)ethyl][1,3]thiazolo[4,5-d]pyrimidine-2,7-diamine,
N⁷-(3-chloro-4-fluorophenyl)-N²-[3-(4-methylpiperazin-1-yl)propyl][1,3]thiazolo[4,5-d]pyrimidine-2, 7-diamine,
N⁷-(3-chloro-4-fluorophenyl)-N²-[3-(dimethylamino)-2,2-dimethylpropyl][1,3]thiazolo[4,5-d]pyrimidine-2,7-diamine,
N⁷-(3-chloro-4-fluorophenyl)-N²-(2-morpholin-4-ylethyl)[1,3]thiazolo[4,5-d]pyrimidine-2,7-diamine,
4-[(3-chloro-4-fluorophenyl)amino]-N-(1-propylpiperidin-4-yl)thieno[2,3-d]pyrimidine-6-carboxamide,
4-[(3-chloro-4-fluorophenyl)amino]-N-(3-pyrolidin-1-ylpropyl)thieno[2,3-d]pyrimidine-6-carboxamide,
4-[(3-chloro-4-fluorophenyl)amino]-N-[3-(dimethylamino)propyl]-N-methylthieno[2,3-d]pyrimidine-6-carboxamide,
4-[(3-chloro-4-fluorophenyl)amino]-N-(2-pyrrolidin-1-ylethyl)thieno[2,3-d]pyrimidine-6-carboxamide,
4-[(3-chloro-4-fluorophenyl)amino]-N-methyl-N-(1-methylpiperidin-4-yl)thieno[2,3-d]pyrimidine-6-carboxamide,
4-[(3-chloro-4-fluorophenyl)amino]-N-(1,2,2,6,6-pentamethylpiperidin-4-yl)thieno[2,3-d]pyrimidine-6-carboxamide,
4-[(3-chloro-4-fluorophenyl)amino]-N-(2-piperidin-1-ylethyl)thieno[2,3-d]pyrimidine-6-carboxamide,
4-(2,3-dihydro-1H-inden-5-ylamino)-N-(1-methylpiperidin-4-yl)thieno[2,3-d]pyrimidine-6-carboxamide,
4-(1,3-dihydro-2-benzofuran-5-ylamino)-N-(1-methylpiperidin-4-yl)thieno[2,3-d]pyrimidine-6-carboxamide,
4-(1H-indol-6-ylamino)-N-(1-methylpiperidin-4-yl)thieno[2,3-d]pyrimidine-6-carboxamide,
4-[(4-chloro-3-fluorophenyl)amino]-N-(1-methylpiperidin-4-yl)thieno[2,3-d]pyrimidine-6-carboxamide,
4-[(3,4-difluorophenyl)amino]-N-(1-methylpiperidin-4-yl)thieno[2,3-d]pyrimidine-6-carboxamide,
4-[(4-fluorophenyl)amino]-N-(1-methylpiperidin-4-yl)thieno[2,3-d]pyrimidine-6-carboxamide,
4-(1,3-benzodioxol-5-ylamino)-N-(1-methylpiperidin-4-yl)thieno[2,3-d]pyrimidine-6-carboxamide,
4-anilino-N-(1-methylpiperidin-4-yl)thieno[2,3-d]pyrimidine-6-carboxamide,
4-[(3-chlorophenyl)amino]-N-(1-methylpiperidin-4-yl)thieno[2,3-d]pyrimidine-6-carboxamide,
4-[(4-chlorophenyl)amino]-N-(1-methylpiperidin-4-yl)thieno[2,3-d]pyrimidine-6-carboxamide,
4-(benzylamino)-N-(1-methylpiperidin-4-yl)thieno[2,3-d]pyrimidine-6-carboxamide,
4-(2,3-dihydro-1-benzofuran-5-ylamino)-N-(1-methylpiperidin-4-yl)thieno[2,3-d]pyrimidine-6-carboxamide,
4-[(3-methoxyphenyl)amino]-N-(1-methylpiperidin-4-yl)thieno[2,3-d]pyrimidine-6-carboxamide,
4-[(3-chloro-4-hydroxyphenyl)amino]-N-(1-methylpiperidin-4-yl)thieno[2,3-d]pyrimidine-6-carboxamide,
4-(3,4-dihydro-2H-1,5-benzodioxepin-7-ylamino)-N-(1-methylpiperidin-4-yl)thieno[2,3-d]pyrimidine-6-carboxamide,
4-[(3-cyano-4-methylphenyl)amino]-N-(1-methylpiperidin-4-yl)thieno[2,3-d]pyrimidine-6-carboxamide,
4-[(4-fluoro-3-methylphenyl)amino]-N-(1-methylpiperidin-4-yl)thieno[2,3-d]pyrimidine-6-carboxamide,
4-[(6-methoxy-2-naphthyl)amino]-N-(1-methylpiperidin-4-yl)thieno[2,3-d]pyrimidine-6-carboxamide,
N-(1-methylpiperidin-4-yl)-4-{[3-(methylthio)phenyl]amino}thieno[2,3-d]pyrimidine-6-carboxamide,
4-[(3-acetylphenyl)amino]-N-(1-methylpiperidin-4-yl)thieno[2,3-d]pyrimidine-6-carboxamide,
4-[(3-ethynylphenyl)amino]-N-(1-methylpiperidin-4-yl)thieno[2,3-d]pyrimidine-6-carboxamide,
4-(1,3-benzothiazol-6-ylamino)-N-(1-methylpiperidin-4-yl)thieno[2,3-d]pyrimidine-6-carboxamide,
4-[(3-chloro-4-fluorophenyl)amino]-N-(1-methylpiperidin-4-yl)thieno[3,2-d]pyrimidine-6-carboxamide,
4-[(3-chloro-4-fluorobenzyl)amino]-N-(1-methylpiperidin-4-yl)thieno[2,3-d]pyrimidine-6-carboxamide,
4-[(3-chloro-4-fluorophenyl)amino]-N-{1-[(1-methyl-1H-pyrrol-2-yl)methyl]piperidin-4-yl}thieno[2,3-d]pyrimidine-6-carboxamide,
4-[(3-chloro-4-fluorophenyl)amino]-N-[1-(2-thienylmethyl)piperidin-4-yl]thieno[2,3-d]pyrimidine-6-carboxamide,
4-[(3-chloro-4-fluorophenyl)amino]-N-[1-(pyridin-3-ylmethyl)piperidin-4-yl]thieno[2,3-d]pyrimidine-6-carboxamide,
4-[(3-chloro-4-fluorophenyl)amino]-N-[1-(quinolin-4-ylmethyl)piperidin-4-yl]thieno[2,3-d]pyrimidine-6-carboxamide,
N-(1-butylpiperidine-4-yl)-4-[(3-chloro-4-fluorophenyl)amino]thieno[2,3-d]pyrimidine-6-carboxamide,
4-[(3-chloro-4-fluorophenyl)amino]-N-[1-(3-methylbut-2-en-1-yl)piperidin-4-yl]thieno[2,3-d]pyrimidine-6-carboxamide,
4-[(3-chloro-4-fluorophenyl)amino]-N-[1-(2-furylmethyl)piperidin-4-yl]thieno[2,3-d]pyrimidine-6-carboxamide,
4-[(3-chloro-4-fluorophenyl)amino]-N-[1-(2-methoxyethyl)piperidin-4-yl]thieno[2,3-d]pyrimidine-6-carboxamide,
4-[(3-chloro-4-fluorophenyl)amino]-N-(1-{[5-(hydroxymethyl)-2-furyl]methyl}piperidin-4-yl)thieno[2,3-d]pyrimidine-6-carboxamide,
4-[(3-chloro-4-fluorophenyl)amino]-N-[1-(2-hydroxybenzyl)piperidin-4-yl]thieno[2,3-d]pyrimidine-6-carboxamide,
2-({4-[({4-[(3-chloro-4-fluorophenyl)amino]thieno[2,3-d]pyrimidin-6-yl}carbonyl)amino]piperidin-1-yl}methyl)benzoic acid,
4-[(3-chloro-4-fluorophenyl)amino]-N-[1-(3-methoxybenzyl)piperidin-4-yl]thieno[2,3-d]pyrimidine-6-carboxamide,
4-[(3-chloro-4-fluorophenyl)amino]-N-[1-(4-cyanobenzyl)piperidin-4-yl]thieno[2,3-d]pyrimidine-6-carboxamide,
4-[(3-chloro-4-fluorophenyl)amino]-N-[1-(1H-imidazol-2-ylmethyl)piperidin-4-yl]thieno[2,3-d]pyrimidine-6-carboxamide,
4-[(3-chloro-4-fluorophenyl)amino]-N-[1-(1H-pyrrol-2-ylmethyl)piperidin-4-yl]thieno[2,3-d]pyrimidine-6-carboxamide,
4-[(3-chlorophenyl)(methyl)amino]-N-(1-methylpiperidin-4-yl)thieno[2,3-d]pyrimidine-6-carboxamide,
4-[(4-chlorophenyl)(methyl)amino]-N-(1-methylpiperidin-4-yl)thieno[2,3-d]pyrimidine-6-carboxamide,
4-[(3-chloro-4-fluorophenyl)amino]-N-[1-(3-cyanobenzyl)piperidin-4-yl]thieno[2,3-d]pyrimidine-6-carboxamide,
ethyl 2-({4-[({4-[(3-chloro-4-fluorophenyl)amino]thieno[2,3-d]pyrimidin-6-yl}carbonyl)amino]piperidin-1-yl}methyl)cyclopropanecarboxylate,
4-[(3-chloro-4-fluorophenyl)amino]-N-[1-(1H-pyrazol-3-ylmethyl)piperidin-4-yl]thieno[2,3-d]pyrimidine-6-carboxamide,
4-[(3-chloro-4-fluorophenyl)amino]-N-{1-[(1-methyl-1H-indol-3-yl)methyl]piperidin-4-yl}thieno[2,3-d]pyrimidine-6-carboxamide,
4-[(3-chloro-4-fluorophenyl)amino]-N-[1-(pyridin-4-ylmethyl)piperidin-4-yl]thieno[2,3-d]pyrimidine-6-carboxamide,
4-[(3-chloro-4-fluorophenyl)amino]-N-[1-(pyridin-2-ylmethyl)piperidin-4-yl]thieno[2,3-d]pyrimidine-6-carboxamide,
4-[(3-chloro-4-fluorophenyl)amino]-N-{1-[4-(methylsulfonyl)benzyl]piperidin-4-yl}thieno[2,3-d]pyrimidine-6-carboxamide,
4-(2,3-dihydro-1H-indol-1-yl)-N-(1-methylpiperidin-4-yl)thieno[2,3-d]pyrimidine-6-carboxamide,
4-(1H-indol-5-ylamino)-N-(1-methylpiperidin-4-yl)thieno[2,3-d]pyrimidine-6-carboxamide,
4-[(3,4-dichlorophenyl)amino]-N-(1-methylpiperidin-4-yl)thieno[2,3-d]pyrimidine-6-carboxamide,
4-[(3-cyanophenyl)amino]-N-(1-methylpiperidin-4-yl)thieno[2,3-d]pyrimidine-6-carboxamide,
N-(1-benzylpiperidin-4-yl)-4-[(3-chloro-4-fluorophenyl)amino]thieno[2,3-d]pyrimidine-6-carboxamide,
N-(1-benzylpiperidin-4-yl)-4-[(4-fluorophenyl)amino]thieno[2,3-d]pyrimidine-6-carboxamide,
4-anilino-N-(1-benzylpiperidin-4-yl)thieno[2,3-d]pyrimidine-6-carboxamide,
4-[(3-chloro-4-fluorophenyl)amino]-N-[1-(cyclopropylmethyl)piperidin-4-yl]thieno[2,3-d]pyrimidine-6-carboxamide,
N-[1-(cyclopropylmethyl)piperidin-4-yl]-4-[(4-fluorophenyl)amino]thieno[2,3-d]pyrimidine-6-carboxamide,
N-[1-(cyclopropylmethyl)piperidin-4-yl]-4-[(3,4-difluorophenyl)amino]thieno[2,3-d]pyrimidine-6-carboxamide,
N-(3-chloro-4-fluorophenyl)-6-({4-[(1-methylpiperidin-3-yl)methyl]piperazin-1-yl}carbonyl)thieno[2,3-d]pyrimidin-4-amine,
N-{4-[(3-chloro-4-fluorophenyl)amino]thieno[2,3-d]pyrimidin-6-yl}-3-pyrrolidin-1-ylpropanamide,
N¹-{4-[(3-chloro-4-fluorophenyl)amino]thieno[2,3-d]pyrimidin-6-yl}-N³-(3-morpholin-4-ylpropyl)-beta-alaninamide,
N-(3-chloro-4-fluorophenyl)-2-[(1-methylpiperidin-3-yl)methoxy][1,3]thiazolo[4,5-d]pyridin-7-amine,
N-(3-chloro-4-fluorophenyl)-2-(2-pyrrolidin-1-ylethoxy)[1,3]thiazolo[4,5-d]pyrimidin-7-amine,
4-[(3-chloro-4-fluorophenyl)amino]-N-(1-methylpiperidin-4-yl)thieno[2,3-d]pyrimidine-6-carboxamide,
N⁷-(4-fluorophenyl)-N²-(2-morpholin-4-ylethyl)[1,3]thiazolo[4,5-d]pyrimidine-2,7-diamine,
N⁷-(4-chlorophenyl)-N²-(2-morpholin-4-ylethyl)[1,3]thiazolo[4,5-d]pyrimidine-2,7-diamine,
N⁷-(2,3-dihydro-1H-inden-5-yl)-N²-(2-morpholin-4-ylethyl)[1,3]thiazolo[4,5-d]pyrimidine-2,7-diamine,
N⁷-(3,4-dichlorophenyl)-N²-(2-morpholin-4-ylethyl)[1,3]thiazolo[4,5-d]pyrimidine-2,7-diamine,
4-[(3-chloro-4-fluorophenyl)amino]-N-(2-morpholin-4-ylethyl)thieno[2,3-d]pyrimidine-6-carboxamide,
4-[(3-chloro-4-fluorophenyl)amino]-N-(3-morpholin-4-ylpropyl)thieno[2,3-d]pyrimidine-6-carboxamide,
4-[(3-chloro-4-fluorophenyl)amino]-N-[2-(diethylamino)ethyl]thieno[2,3-d]pyrimidine-6-carboxamide,
4-[(3-chloro-4-fluorophenyl)amino]-N-[3-(dimethylamino)propyl]thieno[2,3-d]pyrimidine-6-carboxamide,
4-[(3-chloro-4-fluorophenyl)amino]-N-[3-(dimethylamino)-2,2-dimethylpropyl]thieno[2,3-d]pyrimidine-6-carboxamide,
N⁷-phenyl-N²-(2-piperidin-1-ylethyl)[1,3]thiazolo[4,5-d]pyrimidine-2,7-diamine,
N⁷-(3-chlorophenyl)-N²-(2-piperidin-1-ylethyl)[1,3]thiazolo[4,5-d]pyrimidine-2,7-diamine,
N⁷-(3-methylphenyl)-N²-(2-piperidin-1-ylethyl)[1,3]thiazolo[4,5-d]pyrimidine-2,7-diamine,
N⁷-(4-fluorophenyl)-N²-(2-piperidin-1-ylethyl)[1,3]thiazolo[4,5-d]pyrimidine-2,7-diamine,
N⁷-(4-chlorophenyl)-N²-(2-piperidin-1-ylethyl)[1,3]thiazolo[4,5-d]pyrimidine-2,7-diamine,
N⁷-(4-methylphenyl)-N²-(2-piperidin-1-ylethyl)[1,3]thiazolo[4,5-d]pyrimidine-2,7-diamine,
N⁷-(3,4-difluorophenyl)-N²-(2-piperidin-1-ylethyl)[1,3]thiazolo[4,5-d]pyrimidine-2,7-diamine,
7-(3-chloro-4-fluorophenoxy)-N-(2-piperidin-1-ylethyl)[1,3]thiazolo[4,5-d]pyrimidin-2-amine,
N⁷-(2,3-dihydro-1H-inden-5-yl)-N²-(2-piperidin-1-ylethyl)[1,3]thiazolo[4,5-d]pyrimidine-2,7-diamine,
N⁷-(3-chloro-4-fluorophenyl)-N²-(2-piperidin-1-ylethyl)[1,3]thiazolo[4,5-d]pyrimidine-2, 7-diamine,
4-[(3-chloro-4-fluorophenyl)amino]-N-[2-(dimethylamino)ethyl]thieno[2,3-d]pyrimidine-6-carboxamide,
4-[(3-chloro-4-fluorophenyl)amino]-N-{1-[(1-methyl-1H-imidazol-2-yl)methyl]piperidin-4-yl}thieno[2,3-d]pyrimidine-6-carboxamide,
N⁷-(3-chloro-4-fluorophenyl)-5-methyl-N²-(2-piperidin-1-ylethyl)[1,3]thiazolo[4,5-d]pyrimidine-2,7-diamine,
4-[(3-chloro-4-fluorophenyl)amino]-N-{1-[(2-methyl-1H-1-imidazol-4-yl)methyl]piperidin-4-yl}thieno[2,3-d]pyrimidine-6-carboxamide,
N-{1-[(5-chloro-1,3-dimethyl-1H-pyrazol-4-yl)methyl]piperidin-4-yl}-4-[(3-chloro-4-fluorophenyl)amino]thieno[2,3-d]pyrimidine-6-carboxamide,
5-({4-[({4-[(3-chloro-4-fluorophenyl)amino]thieno[2,3-d]pyrimidin-6-yl}carbonyl)amino]piperidin-1-yl}methyl)-2-furoic acid,
4-[(3-chloro-4-fluorophenyl)amino]-N-[1-(2-hydroxyethyl)piperidin-4-yl]thieno[2,3-d]pyrimidine-6-carboxamide,
N-[1-(1,3-benzodioxol-4-ylmethyl)piperidin-4-yl]-4-[(3-chloro-4-fluorophenyl)amino]thieno[2,3-d]pyrimidine-6-carboxamide,
4-[(3-chloro-4-fluorophenyl)amino]-N-{1-[(2-chloropyridin-3-yl)methyl]piperidin-4-yl}thieno[2,3-d]pyrimidine-6-carboxamide,
methyl 6-{4-[({4-[(3-chloro-4-fluorophenyl)amino]thieno[2,3-d]pyrimidin-6-yl}carbonyl)amino]piperidin-1-yl}hexanoate,
4-[(3-chloro-4-fluorophenyl)amino]-N-(1-{[(4S)-2,2-dimethyl-1,3-dioxolan-4-yl]methyl}piperidin-4-yl)thieno[2,3-d]pyrimidine-6-carboxamide,
4-[(3-chloro-4-fluorophenyl)amino]-N-[1-(2,3-dihydro-1H-indol-3-ylmethyl)piperidin-4-yl]thieno[2,3-d]pyrimidine-6-carboxamide,
N-{1-[(2-amino-4-methyl-6-oxo-1,6-dihydropyrimidin-5-yl)methyl]piperidin-4-yl}-4-[(3-chloro-4-fluorophenyl)amino]thieno[2,3-d]pyrimidine-6-carboxamide,
4-[(3-chloro-4-fluorophenyl)amino]-N-(1-{[6-(hydroxymethyl)pyridin-2-yl]methyl}piperidin-4-yl)thieno[2,3-d]pyrimidine-6-carboxamide,
4-[(3-chloro-4-fluorophenyl)amino]-N-{1-[(1-methyl-1H-indol-2-yl)methyl]piperidin-4-yl}thieno[2,3-d]pyrimidine-6-carboxamide,
4-[(3-chloro-4-fluorophenyl)amino]-N-[1-(1H-indol-5-ylmethyl)piperidin-4-yl]thieno[2,3-d]pyrimidine-6-carboxamide,
ethyl 4-({4-[({4-[(3-chloro-4-fluorophenyl)amino]thieno[2,3-d]pyrimidin-6-yl}carbonyl)amino]piperidin-1-yl}methyl)-5-methyl-1H-pyrrole-2-carboxylate,
4-[(3-chloro-4-fluorophenyl)amino]-N-[1-(tetrahydro-2H-pyran-4-ylmethyl)piperidin-4-yl]thieno[2,3-d]pyrimidine-6-carboxamide,
4-[(3-chloro-4-fluorophenyl)amino]-N-[1-(1,3-thiazol-5-ylmethyl)piperidin-4-yl]thieno[2,3-d]pyrimidine-6-carboxamide,
4-[(3-chloro-4-fluorophenyl)amino]-N-{1-[(6-methoxypyridin-3-yl)methyl]piperidin-4-yl}thieno[2,3-d]pyrimidine-6-carboxamide,
4-[(3-chloro-4-fluorophenyl)amino]-N-(1-{[5-cyano-6-(methylthio)pyridin-2-yl]methyl}piperidin-4-yl)thieno[2,3-d]pyrimidine-6-carboxamide,
4-[(3-chloro-4-fluorophenyl)amino]-N-{1-[(1-methyl-1H-imidazol-5-yl)methyl]piperidin-4-yl}thieno[2,3-d]pyrimidine-6-carboxamide,
methyl 3-({4-[({4-[(3-chloro-4-fluorophenyl)amino]thieno[2,3-d]pyrimidin-6-yl}carbonyl)amino]piperidin-1-yl}methyl)-1H-indole-5-carboxylate,
4-[(3-chloro-4-fluorophenyl)amino]-N-[1-(1,3-thiazol-2-ylmethyl)piperidin-4-yl]thieno[2,3-d]pyrimidine-6-carboxamide,
4-({4-[({4-[(3-chloro-4-fluorophenyl)amino]thieno[2,3-d]pyrimidin-6-yl}carbonyl)amino]piperidin-1-yl}methyl)benzoic acid,
4-[(3-chloro-4-fluorophenyl)amino]-N-[1-(1H-imidazol-4-ylmethyl)piperidin-4-yl]thieno[2,3-d]pyrimidine-6-carboxamide,
4-[(3-chloro-4-fluorophenyl)amino]-N-[1-(2-cyanoethyl)piperidin-4-yl]thieno[2,3-d]pyrimidine-6-carboxamide,
4-[(3-chloro-4-fluorophenyl)amino]-N-{1-[(5-methylisoxazol-3-yl)methyl]piperidin-4-yl}thieno[2,3-d]pyrimidine-6-carboxamide,
4-[(3-chloro-4-fluorophenyl)amino]-N-[1-(cyclobutylmethyl)piperidin-4-yl]thieno[2,3-d]pyrimidine-6-carboxamide,
4-[(3-chloro-4-fluorophenyl)amino]-N-[1-(1,2,4-oxadiazol-3-ylmethyl)piperidin-4-yl]thieno[2,3-d]pyrimidine-6-carboxamide,
4-[(3-chloro-4-fluorophenyl)amino]-N-{1-[(1-oxidopyridin-3-yl)methyl]piperidin-4-yl}thieno[2,3-d]pyrimidine-6-carboxamide,
4-[(3-chloro-4-fluorophenyl)amino]-N-{1-[3-(morpholin-4-ylsulfonyl)propyl]piperidin-4-yl}thieno[2,3-d]pyrimidine-6-carboxamide,
4-[(3-chloro-4-fluorophenyl)amino]-N-{1-[(3,5-dimethylisoxazol-4-yl)methyl]piperidin-4-yl}thieno[2,3-d]pyrimidine-6-carboxamide,
4-[(3-chloro-4-fluorophenyl)amino]-N-[1-(thiiran-2-ylmethyl)piperidin-4-yl]thieno[2,3-d]pyrimidine-6-carboxamide,
4-[(3-chloro-4-fluorophenyl)amino]-N-{1-[2-(phenylsulfonyl)ethyl]piperidin-4-yl}thieno[2,3-d]pyrimidine-6-carboxamide,
4-[(3-chloro-4-fluorophenyl)amino]-N-[1-(1,3-thiazol-4-ylmethyl)piperidin-4-yl]thieno[2,3-d]pyrimidine-6-carboxamide,
4-[(4-methoxybenzyl)amino]-N-(1-methylpiperidin-4-yl)thieno[2,3-d]pyrimidine-6-carboxamide,
4-[(3-chloro-4-fluorophenyl)amino]-N-[1-(1-methylprop-2-yn-1-yl)piperidin-4-yl]thieno[2,3-d]pyrimidine-6-carboxamide,
methyl 2-{4-[({4-[(3-chloro-4-fluorophenyl)amino]thieno[2,3-d]pyrimidin-6-yl}carbonyl)amino]piperidin-1-yl}-3-(1H-imidazol-4-yl)propanoate,
4-[(3-chloro-4-fluorophenyl)amino]-N-{1-[2-(diethylamino)-1-methyl-2-oxoethyl]piperidin-4-yl}thieno[2,3-d]pyrimidine-6-carboxamide,
4-[(3-chloro-4-fluorophenyl)amino]-N-{1-[cyano(phenyl)methyl]piperidin-4-yl}thieno[2,3-d]pyrimidine-6-carboxamide,
4-(benzoylamino)-N-(1-methylpiperidin-4-yl)thieno[2,3-d]pyrimidine-6-carboxamide,
4-[(3,4-difluorobenzoyl)amino]-N-(1-methylpiperidin-4-yl)thieno[2,3-d]pyrimidine-6-carboxamide,
4-[(4-chlorobenzoyl)amino]-N-(1-methylpiperidin-4-yl)thieno[2,3-d]pyrimidine-6-carboxamide,
N-(1-benzylpiperidin-4-yl)-4-[(3-cyanophenyl)amino]thieno[2,3-d]pyrimidine-6-carboxamide,
4-[(3-cyano-4-fluorophenyl)amino]-N-(1-methylpiperidin-4-yl)thieno[2,3-d]pyrimidine-6-carboxamide,
N-(1-benzylpiperidin-4-yl)-4-[(3,4-difluorophenyl)amino]thieno[2,3-d]pyrimidine-6-carboxamide,
4-anilino-N-[1-(cyclopropylmethyl)piperidin-4-yl]thieno[2,3-d]pyrimidine-6-carboxamide,
N-(1-benzylazetidin-3-yl)-4-[(3,4-difluorophenyl)amino]thieno[2,3-d]pyrimidine-6-carboxamide,
4-[(3,4-difluorophenyl)amino]-N-(1-propylazetidin-3-yl)thieno[2,3-d]pyrimidine-6-carboxamide,
N-(1-methylpiperidin-4-yl)-4-{[1-(tetrahydro-2H-pyran-2-yl)-1H-indazol-5-yl]amino}thieno[2,3-d]pyrimidine-6-carboxamide,
4-[(1-benzyl-1H-indol-5-yl)amino]-N-(1-methylpiperidin-4-yl)thieno[2,3-d]pyrimidine-6-carboxamide,
N-(1-methylpiperidin-4-yl)-4-{[1-(phenylsulfonyl)-1H-indol-5-yl]amino}thieno[2,3-d]pyrimidine-6-carboxamide,
4-(1H-benzimidazol-5-ylamino)-N-(1-methylpiperidin-4-yl)thieno[2,3-d]pyrimidine-6-carboxamide,
4-(1H-indazol-6-ylamino)-N-(1-methylpiperidin-4-yl)thieno[2,3-d]pyrimidine-6-carboxamide,
4-(1H-indol-5-ylamino)-N-(1-propylpiperidin-4-yl)thieno[2,3-d]pyrimidine-6-carboxamide,
4-(1H-indol-5-ylamino)-N-[1-(2-methoxyethyl)piperidin-4-yl]thieno[2,3-d]pyrimidine-6-carboxamide,
N-(1-benzylpiperidin-4-yl)-4-(1H-indol-5-ylamino)thieno[2,3-d]pyrimidine-6-carboxamide,
4-(3,4-difluorophenoxy)-N-(1-methylpiperidin-4-yl)thieno[2,3-d]pyrimidine-6-carboxamide,
4-(1H-indol-6-ylamino)-N-(1-propylpiperidin-4-yl)thieno[2,3-d]pyrimidine-6-carboxamide,
4-(1H-indol-6-ylamino)-N-[1-(2-methoxyethyl)piperidin-4-yl]thieno[2,3-d]pyrimidine-6-carboxamide,
N-(1-benzylpiperidin-4-yl)-4-(1H-indol-6-ylamino)thieno[2,3-d]pyrimidine-6-carboxamide,
7-[(3-chloro-4-fluorophenyl)amino]-N-(1-methylpiperidin-4-yl)[1,3]thiazolo[5,4-d]pyrimidine-2-carboxamide,
4-[(3-chloro-4-fluorophenyl)amino]-N-[1-(1,2,3-thiadiazol-4-ylmethyl)piperidin-4-yl]thieno[2,3-d]pyrimidine-6-carboxamide,
4-[(3-chloro-4-fluorophenyl)amino]-N-(1-ethylpiperidin-4-yl)thieno[2,3-d]pyrimidine-6-carboxamide,
N-1-azabicyclo[2.2.2]oct-3-yl-4-[(3-chloro-4-fluorophenyl)amino]thieno[2,3-d]pyrimidine-6-carboxamide,
N-(1-benzylpyrrolidin-3-yl)-4-[(3-chloro-4-fluorophenyl)amino]thieno[2,3-d]pyrimidine-6-carboxamide,
4-[(3-chloro-4-fluorophenyl)amino]-N-[(1-ethylpyrrolidin-2-yl)methyl]thieno[2,3-d]pyrimidine-6-carboxamide,
6-[(4-benzylpiperazin-1-yl)carbonyl]-N-(3-chloro-4-fluorophenyl)thieno[2,3-d]pyrimidin-4-amine,
N-(3-chloro-4-fluorophenyl)-6-[(4-methyl-1,4-diazepan-1-yl)carbonyl]thieno[2,3-d]pyrimidin-4-amine,
N-(3-chloro-4-fluorophenyl)-6-[(4-pyrrolidin-1-ylpiperidin-1-yl)carbonyl]thieno[2,3-d]pyrimidin-4-amine,
N-(3-chloro-4-fluorophenyl)-6-{[(3S)-3-(dimethylamino)pyrrolidin-1-yl]carbonyl}thieno[2,3-d]pyrimidin-4-amine,
4-(1H-indol-6-ylamino)-N-(1-methylpiperidin-4-yl)thieno[3,2-d]pyrimidine-6-carboxamide,
4-[(3-chloro-4-fluorophenyl)amino]-N-{1-[(5-fluoro-1H-indol-3-yl)methyl]piperidin-4-yl}thieno[2,3-d]pyrimidine-6-carboxamide,
4-[(3-chloro-4-fluorophenyl)amino]-N-[1-(1H-indol-6-ylmethyl)piperidin-4-yl]thieno[2,3-d]pyrimidine-6-carboxamide,
4-[(3-chloro-4-fluorophenyl)amino]-N-{1-[(1,3-dimethyl-1H-pyrazol-5-yl)methyl]piperidin-4-yl}thieno[2,3-d]pyrimidine-6-carboxamide,
4-[(3-chloro-4-fluorophenyl)amino]-N-[1-(4-methyl-5-oxotetrahydrofuran-2-yl)piperidin-4-yl]thieno[2,3-d]pyrimidine-6-carboxamide,
4-[(3-chloro-4-fluorophenyl)amino]-N-(1-isopropylpiperidin-4-yl)thieno[2,3-d]pyrimidine-6-carboxamide,
4-[(3-chloro-4-fluorophenyl)amino]-N-[1-(1-methyl-2-oxopropyl)piperidin-4-yl]thieno[2,3-d]pyrimidine-6-carboxamide,
4-[(3-chloro-4-fluorophenyl)amino]-N-[1-(1-phenylethyl)piperidin-4-yl]thieno[2,3-d]pyrimidine-6-carboxamide,
4-[(3-cyanophenyl)amino]-N-[1-(1H-imidazol-2-ylmethyl)piperidin-4-yl]thieno[2,3-d]pyrimidine-6-carboxamide,
4-[(3-cyanophenyl)amino]-N-{1-[4-(methylsulfonyl)benzyl]piperidin-4-yl}thieno[2,3-d]pyrimidine-6-carboxamide,
4-[(3-cyanophenyl)amino]-N-[1-(cyclopropylmethyl)piperidin-4-yl]thieno[2,3-d]pyrimidine-6-carboxamide,
4-[(3-cyanophenyl)amino]-N-[1-(2-thienylmethyl)piperidin-4-yl]thieno[2,3-d]pyrimidine-6-carboxamide,
4-[(3-cyanophenyl)amino]-N-(1-{[6-(hydroxymethyl)pyridin-2-yl]methyl}piperidin-4-yl)thieno[2,3-d]pyrimidine-6-carboxamide,
4-[(3-cyanophenyl)amino]-N-{1-[(5-fluoro-1H-indol-3-yl)methyl]piperidin-4-yl}thieno[2,3-d]pyrimidine-6-carboxamide,
4-[(3-cyanophenyl)amino]-N-{1-[(6-methoxypyridin-3-yl)methyl]piperidin-4-yl}thieno[2,3-d]pyrimidine-6-carboxamide,
N-(1-methylpiperidin-4-yl)-4-(quinolin-6-ylamino)thieno[2,3-d]pyrimidine-6-carboxamide,
4-(2,3-dihydro-1,4-benzodioxin-6-ylamino)-N-(1-methylpiperidin-4-yl)thieno[2,3-d]pyrimidine-6-carboxamide,
4-[(2-methyl-1,3-benzothiazol-6-yl)amino]-N-(1-methylpiperidin-4-yl)thieno[2,3-d]pyrimidine-6-carboxamide,
4-[(2-methyl-1H-indol-5-yl)amino]-N-(1-methylpiperidin-4-yl)thieno[2,3-d]pyrimidine-6-carboxamide,
N-(1-methylpiperidin-4-yl)-4-[(2-methylquinolin-6-yl)amino]thieno[2,3-d]pyrimidine-6-carboxamide,
N-[(4-benzylmorpholin-2-yl)methyl]-4-[(3-chloro-4-fluorophenyl)amino]thieno[2,3-d]pyrimidine-6-carboxamide,
N-(3-chloro-4-fluorophenyl)-6-{[4-(dimethylamino)piperidin-1-yl]carbonyl}thieno[2,3-d]pyrimidin-4-amine,
N-(1-benzylpiperidin-4-yl)-4-[(3-chloro-4-fluorophenyl)amino]-N-methylthieno[2,3-d]pyrimidine-6-carboxamide,
6-[(4-benzyl-1,4-diazepan-1-yl)carbonyl]-N-(3-chloro-4-fluorophenyl)thieno[2,3-d]pyrimidin-4-amine,
4-[(3-chloro-4-fluorophenyl)amino]-N-[2-(dimethylamino)-1-methylethyl]thieno[2,3-d]pyrimidine-6-carboxamide,
4-[(3-chloro-4-fluorophenyl)amino]1-N-(2-thiomorpholin-4-ylethyl)thieno[2,3-d]pyrimidine-6-carboxamide,
4-[(2,2-dimethyl-3-oxo-3,4-dihydro-2H-1,4-benzoxazin-6-yl)amino]-N-(1-methylpiperidin-4-yl)thieno[2,3-d]pyrimidine-6-carboxamide,
N-[1-(2-hydroxybenzyl)piperidin-4-yl]-4-(1H-indazol-6-ylamino)thieno[2,3-d]pyrimidine-6-carboxamide,
N-(1-benzylpiperidin-4-yl)-4-(1H-indazol-6-ylamino)thieno[2,3-d]pyrimidine-6-carboxamide,
N-[1-(4-cyanobenzyl)piperidin-4-yl]-4-(1H-indazol-6-ylamino)thieno[2,3-d]pyrimidine-6-carboxamide,
4-(1H-indazol-6-ylamino)-N-{1-[4-(methylsulfonyl)benzyl]piperidin-4-yl}thieno[2,3-d]pyrimidine-6-carboxamide,
4-(1H-indazol-6-ylamino)-N-{1-[(1-methyl-1H-pyrrol-2-yl)methyl]piperidin-4-yl}thieno[2,3-d]pyrimidine-6-carboxamide,
N-[1-(3,4-dimethoxybenzyl)piperidin-4-yl]-4-(1H-indazol-6-ylamino)thieno[2,3-d]pyrimidine-6-carboxamide,
4-(1H-indazol-6-ylamino)-N-[1-(1H-pyrrol-2-ylmethyl)piperidin-4-yl]thieno[2,3-d]pyrimidine-6-carboxamide,
N-(1-{[5-(hydroxymethyl)-2-furyl]methyl}piperidin-4-yl)-4-(1H-indazol-6-ylamino)thieno[2,3-d]pyrimidine-6-carboxamide,
N-[1-(1H-imidazol-2-ylmethyl)piperidin-4-yl]-4-(1H-indazol-6-ylamino)thieno[2,3-d]pyrimidine-6-carboxamide,
4-(1H-indazol-6-ylamino)-N-[1-(1H-indol-3-ylmethyl)piperidin-4-yl]thieno[2,3-d]pyrimidine-6-carboxamide,
4-(1H-indazol-6-ylamino)-N-[1-(pyridin-3-ylmethyl)piperidin-4-yl]thieno[2,3-d]pyrimidine-6-carboxamide,
N-[1-(cyclopropylmethyl)piperidin-4-yl]-4-(1H-indazol-6-ylamino)thieno[2,3-d]pyrimidine-6-carboxamide,
4-(1H-indazol-6-ylamino)-N-[1-(1H-pyrazol-3-ylmethyl)piperidin-4-yl]thieno[2,3-d]pyrimidine-6-carboxamide,
N-[1-(2-hydroxyethyl)piperidin-4-yl]-4-(1H-indazol-6-ylamino)thieno[2,3-d]pyrimidine-6-carboxamide,
N-[1-(2,3-dihydro-1H-indol-3-ylmethyl)piperidin-4-yl]-4-(1H-indazol-6-ylamino)thieno[2,3-d]pyrimidine-6-carboxamide,
N-[1-(1H-imidazol-4-ylmethyl)piperidin-4-yl]-4-(1H-indazol-6-ylamino)thieno[2,3-d]pyrimidine-6-carboxamide,
4-(1H-indazol-6-ylamino)-N-[1-(tetrahydro-2H-pyran-4-ylmethyl)piperidin-4-yl]thieno[2,3-d]pyrimidine-6-carboxamide,
N-(3-chloro-4-fluorophenyl)-6-{[(3R)-3-(dimethylamino)pyrrolidin-1-yl]carbonyl}thieno[2,3-d]pyrimidin-4-amine,
ethyl 1′-{[4-(1H-indol-6-ylamino)thieno[2,3-d]pyrimidin-6-yl]carbonyl}-1,4′-bipiperidine-4-carboxylate,
4-[(3-chloro-4-fluorophenyl)amino]-N-[1-(3-methylbutyl)piperidin-4-yl]thieno[2,3-d]pyrimidine-6-carboxamide,
4-[(3-chloro-4-fluorophenyl)amino]-N-{1-[2-(tetrahydro-2H-pyran-4-yl)ethyl]piperidin-4-yl}thieno[2,3-d]pyrimidine-6-carboxamide,
N-[1-(3-fluoro-2-hydroxybenzyl)piperidin-4-yl]-4-(1H-indazol-6-ylamino)thieno[2,3-d]pyrimidine-6-carboxamide,
N-[1-(2-hydroxy-3-methoxybenzyl)piperidin-4-yl]-4-(1H-indazol-6-ylamino)thieno[2,3-d]pyrimidine-6-carboxamide,
4-(1H-indazol-6-ylamino)-N-[1-(3-phenylbutyl)piperidin-4-yl]thieno[2,3-d]pyrimidine-6-carboxamide,
4-(1H-indazol-6-ylamino)-N-[1-(3-phenylpropyl)piperidin-4-yl]thieno[2,3-d]pyrimidine-6-carboxamide,
N-[1-(3-hydroxybenzyl)piperidin-4-yl]-4-(1H-indazol-6-ylamino)thieno[2,3-d]pyrimidine-6-carboxamide,
4-(1H-indazol-6-ylamino)-N-(1-propylpiperidin-4-yl)thieno[2,3-d]pyrimidine-6-carboxamide,
N-{[(2R)-1-(4-cyanobenzyl)pyrrolidin-2-yl]methyl}-4-(1H-indazol-6-ylamino)thieno[2,3-d]pyrimidine-6-carboxamide,
4-[(3-chloro-4-fluorophenyl)amino]-N-[1-(2-oxopyrrolidin-3-yl)piperidin-4-yl]thieno[2,3-d]pyrimidine-6-carboxamide,
4-[(3-methylcyclohexyl)amino]-N-(1-methylpiperidin-4-yl)thieno[2,3-d]pyrimidine-6-carboxamide,
4-methoxy-N-(1-methylpiperidin-4-yl)thieno[2,3-d]pyrimidine-6-carboxamide and
N-(1-benzylpiperidin-4-yl)-4-methoxythieno[2,3-d]pyrimidine-6-carboxamide,
trans-4-(4-Fluorophenylamino)-thieno[2,3-d]pyrimidine-6-carboxylic acid (1-benzyl-3-hydroxy-piperidin-4-yl)-amide,
N-(1-Benzylpiperidin-4-yl)-2-amino-4-[(4-fluorophenyl)amino]thieno[2,3-d]pyrimidine-6-carboxamide

27. The use according to any one of claims 1 to 12 for the preparation of a medicament for the treatment of a chemokine mediated disease wherein the chemokine binds one or more chemokine receptors.

28. The use according to claim 27 in which the chemokine receptor belongs to the CXC chemokine receptor subfamily.

29. The use according to claim 27 or claim 28 in which the chemokine receptor is the CXR2 receptor.

30. The use according to any one of claims 1 to 12 for the preparation of a medicament for treating an inflammatory disease in a patient suffering from, or at risk of, said disease.

31. The use according to any one of claims 1 to 12 for the preparation of a medicament for treating a disease in which angiogenesis is associated with raised CXCR2 chemokine levels.

32. The use according to any one of claims 1 to 12 for the preparation of a medicament for treating psoriasis.

31. The use according to any one of claims 1 to 12 for the preparation of a medicament for treating COPD.

32. The use according to any one of claims 1 to 12 for the preparation of a medicament for treating cancer.

33. The use according to any one of claims 1 to 12 for the preparation of a medicament for treating disease of the gastrointestinal tract.

34. A compound, salt or solvate according to claim 23 for use in treating a chemokine mediated disease wherein the chemokine binds one or more chemokine receptors.

35. A compound, salt or solvate according to claim 34 in which the chemokine receptor belongs to the CXC chemokine receptor subfamily.

36. A compound, salt or solvate according to claim 34 or claim 35 in which the chemokine receptor is the CXR2 receptor.

37. A compound, salt or solvate as claimed in claim 23 for use in treating an inflammatory disease in a patient suffering from, or at risk of, said disease.

38. A compound, salt or solvate according to claim 23, wherein the disease is, a disease in which angiogenesis is associated with raised CXCR2 chemokine levels.

39. A compound, salt or solvate according to claim 23, wherein the disease is psoriasis.

40. A compound, salt or solvate according to claim 23, wherein the disease is COPD.

41. A compound, salt or solvate according to claim 23, wherein the disease is cancer.

42. A compound, salt or solvate according to claim 23 wherein the disease is of the gastrointestinal tract.
